SEQUENCE LISTING (101 sequences)

SEQ ID NO: 1 - protein, 27 aa, Homo sapiens
FTIKFQEFSPNLWGFEFKKNHDYYIIS

SEQ ID NO: 2 - protein, 14 aa, Homo sapiens
KFQLFSPFLWGFEF

SEQ ID NO: 3 - protein, 15 aa, Homo sapiens
FTIKFQEFSPNLWGL

SEQ ID NO: 4 - protein, 15 aa, Homo sapiens
QEFSPNLWGLEFKKN

SEQ ID NO: 5 - protein, 15 aa, Homo sapiens
NLWGLEFKKNHDYYI

SEQ ID NO: 6 - protein, 15 aa, Homo sapiens
EFKKNHDYYIIATSN

SEQ ID NO: 7 - protein, 27 aa, synthetic construct
  REGION 1..27: note = Synthetic polypeptide
  VARIANT 2..3: note = misc_feature - Xaa can be any naturally occurring amino acid
  VARIANT 7: note = MISC_FEATURE - Xaa is Leu or Glu
  VARIANT 9: note = MISC_FEATURE - Xaa is Ser or Thr
  VARIANT 11: note = MISC_FEATURE - Xaa is Phe or Asn
  VARIANT 12: note = MISC_FEATURE - Xaa is Ser or Leu
  VARIANT 13: note = MISC_FEATURE - Xaa is Xaa or Leu
  VARIANT 15: note = misc_feature - Xaa can be any naturally occurring amino acid
  VARIANT 16: note = MISC_FEATURE - Xaa is Glu or Ala
  VARIANT 18..22: note = misc_feature - Xaa can be any naturally occurring amino acid
  VARIANT 25: note = MISC_FEATURE - Xaa is Ile or Tyr
  VARIANT 27: note = misc_feature - Xaa can be any naturally occurring amino acid
FXXKFQXFXPXXXFXXFXXXXXYYXIX

SEQ ID NO: 8 - skipped

SEQ ID NO: 9 - protein, 110 aa, Homo sapiens
DYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKV

SEQ ID NO: 10 - protein, 111 aa, Homo sapiens
GYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNAVDRPCLRLKV

SEQ ID NO: 11 - protein, 116 aa, Homo sapiens
GYTVQVNVNDYLDIYCPHYNSSGVGPGAGPGPGGGAEQYVLYMVSRNGYRTCNASQGFKRWECNRPHAPHSPIKFSEKFQRYSAFSLGYEFHAGHEYYYISTPTHNLHWKCLRMKV

SEQ ID NO: 12 - protein, 105 aa, Homo sapiens
VVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQV

SEQ ID NO: 13 - protein, 110 aa, Homo sapiens
DYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKV

SEQ ID NO: 14 - protein, 114 aa, Homo sapiens
SGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMK

SEQ ID NO: 15 - protein, 116 aa, Homo sapiens
PGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMK

SEQ ID NO: 16 - protein, 119 aa, Homo sapiens
AEGGYVLYPQIGDRLDLLCPRARPPGPHSSPNYEFYKLYLVGGAQGRRCEAPPAPNLLLTCDRPDLDLRFTIKFQEYSPNLWGHEFRSHHDYYIIATSDGTREGLESLQGGVCLTRGMK

SEQ ID NO: 17 - protein, 28 aa, Homo sapiens
KLSEKFQRFTPFTLGKEFKEGHSYYYIS

SEQ ID NO: 18 - protein, 28 aa, Homo sapiens
KFSEKFQLFTPFSLGFEFRPGHEYYYIS

SEQ ID NO: 19 - protein, 28 aa, Homo sapiens
KFSEKFQRYSAFSLGYEFHAGHEYYYIS

SEQ ID NO: 20 - protein, 28 aa, Homo sapiens
QFSEKIQRFTPFSLGFEFLPGETYYYIS

SEQ ID NO: 21 - protein, 28 aa, Homo sapiens
KFSEKFQLFTPFSLGFEFRPGREYFYIS

SEQ ID NO: 22 - protein, 28 aa, Homo sapiens
RFTIKFQEFSPNYMGLEFKKHHDYYITS

SEQ ID NO: 23 - protein, 28 aa, Homo sapiens
KFTIKFQEFSPNLWGLEFQKNKDYYIIS

SEQ ID NO: 24 - protein, 28 aa, Homo sapiens
RFTIKFQEYSPNLWGHEFRSHHDYYIIA

SEQ ID NO: 25 - protein, 7 aa, Homo sapiens
CKETFNL

SEQ ID NO: 26 - DNA, 21 bp, Homo sapiens
  source 1..21: mol_type = unassigned DNA
tgcaaggagaccttcaacttg

SEQ ID NO: 27 - DNA, 21 bp, Homo sapiens
  source 1..21: mol_type = unassigned DNA
tgcaaggagaccttcaacctc

SEQ ID NO: 28 - DNA, 21 bp, Homo sapiens
  source 1..21: mol_type = unassigned DNA
tgcaaggagaccttcaacctt

SEQ ID NO: 29 - DNA, 21 bp, Homo sapiens
  source 1..21: mol_type = unassigned DNA
tgcaaggagactttcaacctc

SEQ ID NO: 30 - DNA, 21 bp, Homo sapiens
  source 1..21: mol_type = unassigned DNA
tgcaaggagacctttaacctg

SEQ ID NO: 31 - DNA, 21 bp, Homo sapiens
  source 1..21: mol_type = unassigned DNA
tgcaaggagacgtttaacctc

SEQ ID NO: 32 - DNA, 21 bp, Homo sapiens
  source 1..21: mol_type = unassigned DNA
tgtaaggagacatttaacatg

SEQ ID NO: 33 - DNA, 21 bp, Homo sapiens
  source 1..21: mol_type = unassigned DNA
tgtaaagaaacctttaacctg

SEQ ID NO: 34 - DNA, 21 bp, Homo sapiens
  source 1..21: mol_type = unassigned DNA
tgcaaggagaccttcaacctc

SEQ ID NO: 35 - DNA, 21 bp, Homo sapiens
  source 1..21: mol_type = unassigned DNA
tgcaaggagacctttaacctc

SEQ ID NO: 36 - DNA, 21 bp, Homo sapiens
  source 1..21: mol_type = unassigned DNA
tgcaaggagacgtttaacctc

SEQ ID NO: 37 - DNA, 21 bp, Homo sapiens
  source 1..21: mol_type = unassigned DNA
tgcaaggagaccttcaacctg

SEQ ID NO: 38 - protein, 7 aa, Homo sapiens
KFSEKFQ

SEQ ID NO: 39 - DNA, 21 bp, Homo sapiens
  source 1..21: mol_type = unassigned DNA
aagttctcagagaagttccag

SEQ ID NO: 40 - protein, 7 aa, Homo sapiens
FTIKFQE

SEQ ID NO: 41 - DNA, 21 bp, Homo sapiens
  source 1..21: mol_type = unassigned DNA
ttcaccatcaagttccaagaa

SEQ ID NO: 42 - protein, 7 aa, Homo sapiens
CKETFNL

SEQ ID NO: 43 - DNA, 21 bp, Homo sapiens
  source 1..21: mol_type = unassigned DNA
tgcaaggagaccttcaacctc

SEQ ID NO: 44 - protein, 7 aa, Homo sapiens
KIDTIAA

SEQ ID NO: 45 - DNA, 21 bp, Homo sapiens
  source 1..21: mol_type = unassigned DNA
aagattgacaccattgctgct

SEQ ID NO: 46 - protein, 7 aa, Homo sapiens
VNITTNQ

SEQ ID NO: 47 - DNA, 21 bp, Homo sapiens
  source 1..21: mol_type = unassigned DNA
gtgaacatcaccaccaaccaa

SEQ ID NO: 48 - protein, 205 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITHSPQAHDNPQEKRLAADDPEVRVLHSIGHSAAPRLFPLAWTVLLLPLLLLQTP

SEQ ID NO: 49 - protein, 205 aa, Homo sapiens
MEFLWAPLLGLCCSLAAADRHIVFWNSSNPKFREEDYTVHVQLNDYLDIICPHYEDDSVADAAMERYTLYMVEHQEYVACQPQSKDQVRWNCNRPSAKHGPEKLSEKFQRFTPFILGKEFKEGHSYYYISKPIYHQESQCLKLKVTVNGKITHNPQAHVNPQEKRLQADDPEVQVLHSIGYSAAPRLFPLVWAVLLLPLLLLQSQ

SEQ ID NO: 50 - protein, 213 aa, Homo sapiens
MAPAQRPLLPLLLLLLPLPPPPFARAEDAARANSDRYAVYWNRSNPRFHAGAGDDGGGYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNAVDRPCLRLKVYVRPTNETLYEAPEPIFTSNNSCSSPGGCRLFLSTIPVLWTLLGS

SEQ ID NO: 51 - protein, 209 aa, Homo sapiens
MAPAQRPLLPLLLLLLPLRARNEDPARANADRYAVYWNRSNPRFQVSAVGDGGGYTVEVSINDYLDIYCPHYGAPLPPAERMERYILYMVNGEGHASCDHRQRGFKRWECNRPAAPGGPLKFSEKFQLFTPFSLGFEFRPGHEYYYISATPPNLVDRPCLRLKVYVRPTNETLYEAPEPIFTSNSSCSGLGGCHLFLTTVPVLWSLLGS

SEQ ID NO: 52 - protein, 191 aa, Homo sapiens
MAAAPLLLLLLLVPVPLLPLLAQGPGGALGNRHAVYWNSSNQHLRREGYTVQVNVNDYLDIYCPHYNSSGVGPGAGPGPGGGAEQYVLYMVSRNGYRTCNASQGFKRWECNRPHAPHSPIKFSEKFQRYSAFSLGYEFHAGHEYYYISTPTHNLHWKCLRMKVSISGTSPKREHLPLAVGIAFFLMTFLAS

SEQ ID NO: 53 - protein, 230 aa, Homo sapiens
MAAAPLLLLLLLVPVPLLPLLAQGPGGALGNRHAVYWNSSNQHLRREGYTVQVNVNDYLDIYCPHYNSSGPGGGAEQYVLYMVNLSGYRTCNASQGSKRWECNRQHASHSPIKFSEKFQRYSAFSLGYEFHAGQEYYYISTPTHNLHWKCLRMKVFVCCASTSHSGEKPVPTLPQFTMGPNVKINVLEDFEGENPQVPKLEKSISGTSPKREHLPLAVGIAFFLMTLLAS

SEQ ID NO: 54 - protein, 201 aa, Homo sapiens
MRLLPLLRTVLWAAFLGSPLRGGSSLRHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQVSVCCKERKSESAHPVGSPGESGTSGWRGGDTPSPLCLLLLLLLLILRLLRIL

SEQ ID NO: 55 - protein, 206 aa, Homo sapiens
MRLLPLLRTVLWAALLGSRLPGCSSLRHPIYWNSSNPRLLRGDAVVELGFNDYLDIFCPHYESPGPPEGPETFALYMVDWSGYEACTAEGANAFQRWNCSMPFAPFSPVRFSEKIQRYTPFPLGFEFLPGETYYYISVPTPESPGRCLRLQVSVCCKESGSSHESAHPVGSPGESGTSGWRGGHAPSPLCLLLLLLLPILRLLRVL

SEQ ID NO: 56 - protein, 228 aa, Homo sapiens
MLHVEMLTLVFLVLWMCVFSQDPGSKAVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 57 - protein, 228 aa, Homo sapiens
MLHVEMLTLLFLVLWMCVFSQDPGSKVVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 58 - protein, 346 aa, Homo sapiens
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 59 - protein, 345 aa, Homo sapiens
MARPGQRWLSKWLVAMVVLTLCRLATPLAKNLEPVSWSSLNPKFLSGKGLVIYPKIGDKLDIICPRAEAGRPYEYYKLYLVRPEQAAACSTVLDPNVLVTCNKPHQEIRFTIKFQEFSPNYMGLEFKKYHDYYITSTSNGSLEGLENREGGVCRTRTMKIVMKVGQDPNAVTPEQLTTSRPSKESDNTVKTATQAPGRGSQGDSDGKHETVNQEEKSGPGAGGGGSGDSDSFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 60 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 61 - protein, 336 aa, Homo sapiens
MAMARSRRDSVWKYCWGLLMVLCRTAISRSIVLEPIYWNSSNSKFLPGQGLVLYPQIGDKLDIICPKVDSKTVGQYEYYKVYMVDKDQADRCTIKKENTPLLNCARPDQDVKFTIKFQEFSPNLWGLEFQKNKDYYIISTSNGSLEGLDNQEGGVCQTRAMKILMKVGQDASSAGSARNHGPTRRPELEAGTNGRSSTTSPFVKPNPGSSTDGNSAGHSGNNLLGSEVALFAGIASGCIIFIVIIITLVVLLLKYRRRHRKHSPQHTTTLSLSTLATPKRGGNNNGSEPSDVIIPLRTADSVFCPHYEKVSGDYGHPVYIVQEMPPQSPANIYYKV

SEQ ID NO: 62 - protein, 340 aa, Homo sapiens
MGPPHSGPGGVRVGALLLLGVLGLVSGLSLEPVYWNSANKRFQAEGGYVLYPQIGDRLDLLCPRARPPGPHSSPNYEFYKLYLVGGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSLEPGKENLPGDPTSNATSRGAEGPLPPPSMPAVAGAAGGLALLLLGVAGAGGAMCWRRRRAKPSESRHPGPGSFGRGGSLGLGGGGGMGPREAEPGELGIALRGGGAADPPFCPHYEKVSGDYGHPVYIVQDGPPQSPPNIYYKV

SEQ ID NO: 63 - protein, 340 aa, Homo sapiens
MGAPHFGPGGVQVGALLLLGFAGLVSGLSLEPVYWNSANKRFQAEGGYVLYPQIGDRLDLLCPRARPPGPHSSPSYEFYKLYLVEGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSAEPGRDTIPGDPSSNATSRGAEGPLPPPSMPAVAGAAGGMALLLLGVAGAGGAMCWRRRRAKPSESRHPGPGSFGRGGSLGLGGGGGMGPREAEPGELGIALRGGGTADPPFCPHYEKVSGDYGHPVYIVQDGPPQSPPNIYYKV

SEQ ID NO: 64 - protein, 28 aa, Homo sapiens
KLSEKFQRFTPFTLGKEFKEGHSYYYIS

SEQ ID NO: 65 - protein, 28 aa, Homo sapiens
KLSEKFQRFTPFILGKEFKEGHSYYYIS

SEQ ID NO: 66 - protein, 28 aa, Homo sapiens
KFSEKFQLFTPFSLGFEFRPGHEYYYIS

SEQ ID NO: 67 - protein, 28 aa, Homo sapiens
KFSEKFQRYSAFSLGYEFHAGHEYYYIS

SEQ ID NO: 68 - protein, 28 aa, Homo sapiens
KFSEKFQRYSAFSLGYEFHAGQEYYYIS

SEQ ID NO: 69 - protein, 28 aa, Homo sapiens
QFSEKIQRFTPFSLGFEFLPGETYYYIS

SEQ ID NO: 70 - protein, 28 aa, Homo sapiens
RFSEKIQRYTPFPLGFEFLPGETYYYIS

SEQ ID NO: 71 - protein, 28 aa, Homo sapiens
KFSEKFQLFTPFSLGFEFRPGREYFYIS

SEQ ID NO: 72 - protein, 28 aa, Homo sapiens
RFTIKFQEFSPNYMGLEFKKHHDYYITS

SEQ ID NO: 73 - protein, 28 aa, Homo sapiens
RFTIKFQEFSPNYMGLEFKKYHDYYITS

SEQ ID NO: 74 - protein, 28 aa, Homo sapiens
KFTIKFQEFSPNLWGLEFQKNKDYYIIS

SEQ ID NO: 75 - protein, 28 aa, Homo sapiens
RFTIKFQEYSPNLWGHEFRSHHDYYIIA

SEQ ID NO: 76 - protein, 14 aa, Homo sapiens
FQEFSPNLWGLEFQ

SEQ ID NO: 77 - protein, 12 aa, Homo sapiens
QLFTPFSLGFEF

SEQ ID NO: 78 - protein, 14 aa, Homo sapiens
FTPFSLGFEFRPGR

SEQ ID NO: 79 - protein, 18 aa, Homo sapiens
FTIKFQEFSPNLWGLEFQ

SEQ ID NO: 80 - protein, 14 aa, Homo sapiens
KFQLFSPFLWGFEF

SEQ ID NO: 81 - protein, 14 aa, Homo sapiens
KFQLFSPALWGFEF

SEQ ID NO: 82 - protein, 14 aa, Homo sapiens
KFQLFSPFEWGFEF

SEQ ID NO: 83 - protein, 14 aa, Homo sapiens
EFQLFSPFLWGFEF

SEQ ID NO: 84 - protein, 15 aa, Homo sapiens
KFQLFSPFLWFGFAL

SEQ ID NO: 85 - protein, 11 aa, Homo sapiens
KFAAKFQAFAP

SEQ ID NO: 86 - protein, 11 aa, Homo sapiens
AFLWGYEFHAG

SEQ ID NO: 87 - protein, 8 aa, Homo sapiens
WGLEFKKN

SEQ ID NO: 88 - protein, 8 aa, Homo sapiens
WGYEFHAG

SEQ ID NO: 89 - protein, 11 aa, Homo sapiens
AFWWGYEFHAG

SEQ ID NO: 90 - protein, 10 aa, Homo sapiens
AFLWGYEFHA

SEQ ID NO: 91 - protein, 12 aa, Homo sapiens
AFFWWGYEFHAG

SEQ ID NO: 92 - protein, 12 aa, Homo sapiens
AFFWWGYEFHAN

SEQ ID NO: 93 - protein, 12 aa, Homo sapiens
AFFWWGYEFHAK

SEQ ID NO: 94 - protein, 12 aa, Homo sapiens
AFFWKGYEFHAK

SEQ ID NO: 95 - protein, 11 aa, Homo sapiens
AFFWWGYEFHA

SEQ ID NO: 96 - protein, 20 aa, Homo sapiens
YSAFSLGYEFHAGHEYYYIS

SEQ ID NO: 97 - protein, 11 aa, Homo sapiens
AFSLGYEFHAG

SEQ ID NO: 98 - protein, 20 aa, Homo sapiens
FSPNYMGLEFKKHHDYYITS

SEQ ID NO: 99 - protein, 12 aa, Homo sapiens
SPNYMGLEFKKH

SEQ ID NO: 100 - protein, 208 aa, Homo sapiens
MVSAIVLYVLLAAAAHSAFAAMVHHHHHHSAEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRK

SEQ ID NO: 101 - protein, 986 aa, Homo sapiens
MALRRLGAALLLLPLLAAVEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEV

(19) United States
(12) Patent Application Publication
(10) Pub. No.: US 2025/0042968 A1
(43) Pub. Date: Feb. 6, 2025

(54) EPHRIN LIGAND MIMETIC PEPTIDES FOR THE TREATMENT OF NEURODEGENERATIVE DISEASES

(71) Applicant: University of Miami, Miami, FL (US)

(72) Inventor: Daniel PELAEZ, Doral, FL (US)

(21) Appl. No.: 18/922,052

(22) Filed: Oct. 21, 2024

Related U.S. Application Data
(63) Continuation of application No. PCT/US2023/066035, filed on Apr. 20, 2023.
(60) Provisional application No. 63/333,759, filed on Apr. 22, 2022.

(51) Int. Cl. C07K 14/705; A61K 38/00; A61P 27/06

(52) U.S. Cl. CPC C07K 14/705; A61P 27/06; A61K 38/00

(57) ABSTRACT

The present disclosure relates to ephrin ligand mimetic peptides and methods of treating neurodegenerative diseases, using the mimetic peptides.

### CROSS-REFERENCE

This application is a continuation of International Application No. PCT/US2023/066035, filed Apr. 20, 2023, which claims the benefit of U.S. Provisional Application Ser. No. 63/333,759, filed Apr. 22, 2022, which is hereby incorporated by reference in its entirety.

### STATEMENT OF FEDERALLY SPONSORED RESEARCH

This invention was made with government support under grant number P30EY014801, awarded by the National Institutes of Health (NIH). The government has certain rights in the invention.

### INCORPORATION BY REFERENCE OF SEQUENCE LISTING

The present application is being filed along with a Sequence Listing in electronic format. The Sequence Listing is provided as a file entitled 63383_701_301_SL.xml, created Jun. 1, 2023, which is 98,780 bytes in size. The information in the electronic format of the Sequence Listing is incorporated by reference in its entirety.

### FIELD OF THE INVENTION

The present disclosure relates to novel ephrin ligand mimetic peptides and methods of treating neurodegenerative diseases, using the mimetic peptides.

### BACKGROUND

Eph/ephrin signaling is critical for development of the central nervous system (CNS) and is the main axonal guidance cue for topographic mapping of retinal projections onto the superior colliculus in the brain. However, axonal guidance through Eph receptors (forward signaling) is mediated by repulsion, rather than attraction of neuronal processes. Activated Eph-forward signaling leads to axonal growth cone collapse, synaptic instability, and synaptic retraction in CNS neurons. Recently, activation of several Eph receptors has been implicated early in the neurodegenerative processes of several CNS pathologies including Alzheimer's disease (AD), traumatic brain injury (TBI), ischemia-reperfusion events (stroke), and optic neuropathies like glaucoma, making them a valuable target to address neuropathic progression in the CNS. Targeting Eph receptor activation, however, constitutes a major challenge in molecular biology.

Eph receptors and ephrin ligands constitute the largest family of receptor tyrosine kinases (RTK) in mammalian biology because of the size of the protein family. There are significant redundant and compensatory mechanisms in different receptor's biological activity, there is a high level of promiscuity in ligand-receptor interactions, and multiple receptors are involved in any given CNS pathological state. The methods and compositions disclosed herein address these and other needs.

### SUMMARY

Disclosed herein, in some embodiments, are engineered proteins. The engineered protein may comprise the amino acid sequence: ASN-Xaa1-TRP-GLY-Xaa2-GLU-PHE-LYS-Xaa3-Xaa4-HIS-Xaa5-TYR-Xaa6-ILE, wherein Xaa1 is THR, SER TYR, or LEU; Xaa2 is LYS, PHE, TYR, LEU, or HIS; Xaa3 is GLU, PRO, ALA, LYS, or SER; Xaa4 is GLY, HIS, or ASN; Xaa5 is SER, GLU, THR, or ASP; and Xaa6 is TYR or PHE. In some embodiments, the amino acid sequence is at least 80% identical, or at least 90% identical to SEQ ID NO: 5. In some embodiments, the amino acid sequence is identical to SEQ ID NO: 5. In some embodiments, the engineered protein is 10-50 amino acids long. In some embodiments, the engineered protein is 12-20 amino acids long. In some embodiments, the engineered protein is about 15 amino acids long. In some embodiments, the engineered protein is 15 amino acids long. In some embodiments, the engineered protein binds to an ephrin receptor. The engineered protein may comprise an amino acid sequence at least 80% identical, or at least 90% identical to any one of SEQ ID NOs: 1-6. In some embodiments, the amino acid sequence is identical to any one of SEQ ID NOs: 1-6. In some embodiments, the engineered protein is 10-50 amino acids long. In some embodiments, the engineered protein is 12-20 amino acids long. In some embodiments, the engineered protein is about 15 amino acids long. In some embodiments, the engineered protein is 15 amino acids long. In some embodiments, the engineered protein binds to an ephrin receptor. Some embodiments include a pharmaceutical composition. The pharmaceutical composition may include the engineered protein, and a pharmaceutically acceptable carrier. Some embodiments include a method of treatment. The method may include administering the engineered protein to a subject. The method may include administering the pharmaceutical composition to the subject. In some embodiments, the pharmaceutical composition, when administered in an effective amount, treats a neurodegenerative disorder in the subject. In some embodiments, the pharmaceutical composition, when administered in an effective amount, treats an eye disorder in the subject. In some embodiments, the eye disorder comprises glaucoma. In some embodiments, the pharmaceutical composition, when administered in an effective amount, reduces intraocular pressure in an eye of the subject. In some embodiments, the intraocular pressure is reduced by at least 10%, relative to a baseline intraocular pressure measurement. In some embodiments, the pharmaceutical composition, when administered in an effective amount, slows the rate of progression of visual acuity decline in a statistically significant manner. Any treatment effect disclosed herein may be affected by administration of the pharmaceutical composition, or by administration of the engineered protein.

The disclosure provides novel ephrin ligand mimetic peptides and methods of treating neurodegenerative diseases, using the mimetic peptides.

In various embodiments, the disclosure provides methods of treating or preventing a neurodegenerative disease in a subject comprising administering a therapeutically effective amount of one or more pan-Eph receptor Ephrin ligand mimetic peptides to treat or prevent neurodegenerative disease in the subject. In related embodiments, the neurodegenerative disease is Alzheimer's disease (AD), traumatic brain injury (TBI), spinal cord injury (SCI), neuropathy, retinopathy, optic neuropathy, glaucoma/glaucomatous degeneration of the optic nerve and retina, stroke or other Central Nervous System (CNS) neurodegenerative disease. The disclosure also provides methods of treating or preventing cancer in a subject comprising administering a therapeutically effective amount of one or more pan-Eph receptor Ephrin ligand mimetic peptides to treat or prevent cancer in the subject.

In various embodiments the subject is a mammal. In some embodiments, the subject is mouse. In various embodiments, the subject is human.

In various embodiments, the Ephrin ligand mimetic peptide competitively targets ligand binding sites on the Eph receptor. In various embodiments, the Ephrin ligand mimetic peptide mimics receptor binding domains (RBD) in Ephrin (efn) ligands to compete with the binding of Efn ligands. In various embodiments, the Ephrin ligand mimetic peptide antagonizes the activation of both EphA and EphB class receptors. In various embodiments, the Ephrin ligand mimetic peptide increases regeneration of injured axonal projections by >500 μm distal to a crush site on a nerve. In various embodiments, the Ephrin ligand mimetic peptide reduces expression of one or more Eph receptors.

In various embodiments, the disclosure provides compositions comprising a pan-Eph receptor Ephrin ligand mimetic peptide. In various embodiments, the Ephrin ligand mimetic peptide targets ligand binding sites on the Eph receptors by conformational homology to the receptor binding domains (RBD) in Ephrin (efn) ligands. In various embodiments, the Ephrin ligand mimetic peptide mimics the G-H loop of the RBD of efn ligands. In related embodiments, the G-H loop comprises the amino acid sequence F-x-x-K-F-Q-(L/E)-F-(S/T)-P-(F/N)-(S/L)-(x/L)-G-x-(E/A)-F-x-x-x-x-x-Y-Y-(Y/I)-I-S [SEQ ID NO: 7]. In various embodiments, the Ephrin ligand mimetic peptide antagonizes both EphA and EphB receptor activation. In various embodiments, the Ephrin ligand mimetic peptide reduces expression of one or more Eph receptors. In various embodiments, the Ephrin ligand mimetic peptide sequence is SEQ ID NO: 1, SEQ ID NO: 2, SEQ ID NO: 3, SEQ ID NO: 4, SEQ ID NO: 5, or SEQ ID NO: 6.

The present disclosure provides in vivo modified protein comprising: an ephrin receptor bound to a peptide ligand, wherein the ephrin receptor is bound to any amino acid listed in Table 2. In some embodiments, the ephrin receptor comprises an amino acid sequence of SEQ ID NO: 100 or 101. In some embodiments, the binding comprises an interaction type listed in Table 2. In some embodiments, the peptide ligand is bound to any amino acid listed in Table 2. In some embodiments, the interaction type of bonding between the peptide ligand and the ephrin receptor is hydrogen bonding. In some embodiments, the interaction type of bonding between the peptide ligand and the ephrin receptor is ionic bonding. In some embodiments, a peptide ligand atom bound to the ephrin receptor is a component of an NH side chain. In some embodiments, a peptide ligand atom bound to the ephrin receptor is a component of an NH backbone.

In some embodiments, a peptide ligand atom bound to the ephrin receptor is a component of an NH indole group. In some embodiments, a peptide ligand atom bound to the ephrin receptor is a component of an Oe2 side chain. In some embodiments, a peptide ligand atom bound to the ephrin receptor is a component of an NH2+ group. In some embodiments, a peptide ligand atom bound to the ephrin receptor is a component of an NE2-H indole group. In some embodiments, a peptide ligand atom bound to the ephrin receptor is a component of an OH-phenol group. In some embodiments, a peptide ligand atom bound to the ephrin receptor is a component of a C-terminal OH group. In some embodiments, the peptide ligand comprises an amino acid sequence of ASN-Xaa1-TRP-GLY-Xaa2-GLU-PHE-LYS-Xaa3-Xaa4-HIS-Xaa5-TYR-Xaa6-ILE; wherein Xaa1 is THR, SER, TYR, or LEU; wherein Xaa2 is LYS, PHE, TYR, LEU, or HIS; wherein Xaa3 is GLU, PRO, ALA, LYS, or SER; wherein Xaa4 is GLY, HIS, or ASN; wherein Xaa5 is SER, GLU, THR, or ASP; and wherein Xaa6 is TYR or PHE. In some embodiments, an amino acid ASN at an aligned position 1 of SEQ ID NO: 5 in the peptide ligand interacts with a VAL residue in an EphB2 receptor at an aligned position of VAL94 from SEQ ID NO: 101. In some embodiments, an amino acid ASN at an aligned position 1 of SEQ ID NO: 5 in the peptide ligand interacts with a VAL residue in an EphB2 receptor at an aligned position of VAL156 from SEQ ID NO: 101. In some embodiments, an amino acid TRP at an aligned position 3 of SEQ ID NO: 5 in the peptide ligand interacts with a SER residue in an EphB2 receptor at an aligned position of SER47 from SEQ ID NO: 101. In some embodiments, an amino acid GLU at an aligned position 6 of SEQ ID NO: 5 in the peptide ligand interacts with a ARG residue in an EphB2 receptor at an aligned position of ARG155 from SEQ ID NO: 101. In some embodiments, an amino acid LYS at an aligned position 8 of SEQ ID NO: 5 in the peptide ligand interacts with a GLU residue in an EphB2 receptor at an aligned position of GLU44 from SEQ ID NO: 101. In some embodiments, an amino acid HIS at an aligned position 11 of SEQ ID NO: 5 in the peptide ligand interacts with a GLU residue in an EphB2 receptor at an aligned position of GLU44 from SEQ ID NO: 101. In some embodiments, an amino acid TYR at an aligned position 13 of SEQ ID NO: 5 in the peptide ligand interacts with a GLU residue in an EphB2 receptor at an aligned position of GLU66 from SEQ ID NO: 101. In some embodiments, an amino acid ILE at an aligned position 15 of SEQ ID NO: 5 in the peptide ligand interacts with a CYS residue in an EphB2 receptor at an aligned position of CYS62 from SEQ ID NO: 101. In some embodiments, an amino acid ILE at an aligned position 15 of SEQ ID NO: 5 in the peptide ligand interacts with a PHE residue in an EphB2 receptor at an aligned position of PHE65 from SEQ ID NO: 101. In some embodiments, the peptide ligand forms an in vivo bound protein complex by binding to one or more ephrin receptors. In some embodiments, the peptide ligand antagonizes both EphA and EphB receptor activation. In some embodiments, the peptide ligand mimics one or more receptor binding domains (RBD) in one or more ephrin ligands to compete with the binding of natural ephrin ligands. In some embodiments, the peptide ligand functions as an antagonist to EphA2, EphB2 and EphB3. In some embodiments, the peptide ligand functions as an antagonist to EphA2, and EphB2. In some embodiments, the peptide ligand reduces an extent of phosphorylation of one or more Eph receptors following administering of a composition comprising the peptide ligand to a subject. In some embodiments, the peptide ligand reduces expression of one or more Eph receptors following administering of a composition comprising the peptide ligand to a subject. In some embodiments, the peptide ligand increases regeneration of axonal projections by >500 μm distal to a crush site on a nerve following administering of a composition comprising the peptide ligand to a subject. In some embodiments, the peptide ligand induces neurite sprouting following administering of a composition comprising the peptide ligand to a subject. In some embodiments, the peptide ligand protects a plurality of neurons from neurodegeneration following administering of the composition to a subject following administering of a composition comprising the peptide ligand to a subject. In some embodiments, the peptide ligand comprises an amino acid sequence at least 90% identical to a sequence selected from SEQ ID NOs: 1-6. In some embodiments, the peptide ligand comprises an amino acid sequence at least 90% identical to a sequence selected from SEQ ID NOs: 4-6. In some embodiments, the peptide ligand comprises an amino acid sequence at least 90% identical to SEQ ID NO: 5. In some embodiments, the peptide ligand comprises an amino acid sequence 100% identical to SEQ ID NO: 5. In an aspect, are pharmaceutical composition comprising an in vivo modified protein described herein, and a pharmaceutically acceptable carrier. In an aspect, are methods of treating a neurodegenerative condition, comprising administering an effective amount of a pharmaceutical composition described herein to a subject in need thereof. In some embodiments of the methods described herein, the neurodegenerative condition comprises Alzheimer's disease (AD), traumatic brain injury (TBI), spinal cord injury (SCI), neuropathy, retinopathy, optic neuropathy, glaucoma, glaucomatous degeneration of the optic nerve and retina, age-related macular degeneration (AMD), or stroke. In some embodiments of the methods described herein, the neurodegenerative condition comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, or Hallevorden-Spatz disease. In some embodiments of the methods described herein, the neurodegenerative condition comprises Alzheimer's disease (AD). In some embodiments of the methods described herein, the neurodegenerative condition comprises glaucoma. In some embodiments of the methods described herein, one or more symptoms of the neurodegenerative condition of the subject is improved following the administering. In some embodiments of the methods described herein, one or more symptoms of the neurodegenerative condition of the subject is prevented from progressing following the administering. In some embodiments of the methods described herein, one or more symptoms of the neurodegenerative condition of the subject is slowed from progressing following the administering.

The present disclosure provides compositions comprising an ephrin ligand mimetic peptide. In some aspects, the ephrin ligand mimetic peptide comprises an amino acid sequence at least 90% identical to a sequence selected from SEQ ID NOs: 1-6. In some embodiments, the ephrin ligand mimetic peptide comprises an amino acid sequence having one amino acid addition from a sequence selected from SEQ ID NOs: 1-6. In some embodiments, the ephrin ligand mimetic peptide comprises an amino acid sequence having one amino acid deletion from a sequence selected from SEQ ID NOs: 1-6. In some embodiments, the ephrin ligand mimetic peptide comprises an amino acid sequence having one or two amino acid substitutions from a sequence selected from SEQ ID NOs: 1-6. In some embodiments, the ephrin ligand mimetic peptide comprises an amino acid sequence selected from SEQ ID NOs: 1-6. In some embodiments, the ephrin ligand mimetic peptide comprises an amino acid sequence having one or two amino acid substitutions from a sequence selected from SEQ ID NOs: 1-2, or 4-6. In some embodiments, the ephrin ligand mimetic peptide comprises an amino acid sequence having one amino acid addition from a sequence selected from SEQ ID NOs: 1-2, or 4-6. In some embodiments, the ephrin ligand mimetic peptide comprises an amino acid sequence having one amino acid deletion from a sequence selected from SEQ ID NOs: 1-2, or 4-6. In some embodiments, the ephrin ligand mimetic peptide comprises the amino acid sequence selected from SEQ ID NOs: 1-2, or 4-6. In some embodiments, the ephrin ligand mimetic peptide comprises an amino acid sequence having one amino acid addition from a sequence selected from SEQ ID NOs: 4-6. In some embodiments, the ephrin ligand mimetic peptide comprises an amino acid sequence having one amino acid deletion from a sequence selected from SEQ ID NOs: 4-6. In some embodiments, the ephrin ligand mimetic peptide comprises the amino acid sequence selected from SEQ ID NOs: 4-6. In some embodiments, the ephrin ligand mimetic peptide comprises the amino acid sequence of SEQ ID NO: 1. In some embodiments, the ephrin ligand mimetic peptide comprises the amino acid sequence of SEQ ID NO: 2. In some embodiments, the ephrin ligand mimetic peptide comprises the amino acid sequence of SEQ ID NO: 3. In some embodiments, the ephrin ligand mimetic peptide comprises the amino acid sequence of SEQ ID NO: 4. In some embodiments, the ephrin ligand mimetic peptide comprises the amino acid sequence of SEQ ID NO: 5. In some embodiments, the ephrin ligand mimetic peptide comprises the amino acid sequence of SEQ ID NO: 6. In some embodiments, the ephrin ligand mimetic peptide consists essentially of the amino acid sequence of SEQ ID NO: 1. In some embodiments, the ephrin ligand mimetic peptide consists essentially of the amino acid sequence of SEQ ID NO: 2. In some embodiments, the ephrin ligand mimetic peptide consists essentially of the amino acid sequence of SEQ ID NO: 3. In some embodiments, the ephrin ligand mimetic peptide consists essentially of the amino acid sequence of SEQ ID NO: 4. In some embodiments, the ephrin ligand mimetic peptide consists essentially of the amino acid sequence of SEQ ID NO: 5. In some embodiments, the ephrin ligand mimetic peptide consists essentially of the amino acid sequence of SEQ ID NO: 6. In some embodiments, the ephrin ligand mimetic peptide targets a ligand binding site on one or more Eph receptors. In some embodiments, the ephrin ligand mimetic peptide targets a plurality of ligand binding sites on one or more Eph receptors. In some embodiments, the ephrin ligand mimetic peptide competitively targets one or more ligand binding sites on one or more Eph receptors. In some embodiments, competitive targeting comprises ephrin ligand mimetic peptide binding to one or more Eph receptors with a higher binding affinity than a natural ephrin ligand. In some embodiments, the ephrin ligand mimetic peptide binds to EphB2 at any of GLU44, SER47, CYS62, PHE65, GLU66, VAL94, ARG155, or VAL156 in relation to SEQ ID NO: 101. In some embodiments, the natural ephrin ligand is an ephrin-A ligand and wherein the one or more Eph receptors is selected from the group consisting of EphA1, EphA2, EphA3, EphA4, EphA5, EphA6, EphA7, EphA8, and EphA10. In some embodiments, the natural ephrin ligand is an ephrin-B ligand and wherein the one or more Eph receptors is selected from the group consisting of EphB1, EphB2, EphB3, EphB4, and EphB6. In some embodiments, the ephrin-A ligand is ephrin-AT, ephrin-A2, ephrin-A3, ephrin-A4, or ephrin-A5. In some embodiments, the ephrin-A ligand is human ephrin-AT, human ephrin-A2, human ephrin-A3, human ephrin-A4, or human ephrin-A5. In some embodiments, the ephrin-B ligand is ephrin-B1, ephrin-B2, or ephrin-B3. In some embodiments, the ephrin-B ligand is human ephrin-B1, human ephrin-B2, or human ephrin-B3. In some embodiments, the ephrin ligand mimetic peptide mimics one or more receptor binding domains (RBD) in one or more ephrin ligands to compete with the binding of natural ephrin ligands. In some embodiments, the ephrin ligand mimetic peptide mimics one or more receptor binding domains (RBD) found in hEfnA1, hEfnA2, hEfnA3, hEfnA4, hEfnA5, hEfnB1, hEfnB2, or hEfnB3. In some embodiments, the ephrin ligand mimetic peptide mimics two or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics three or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics four or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics receptor binding domains (RBD) found in hEfnA1, hEfnA2, hEfnA3, hEfnA4, hEfnA5, hEfnB1, hEfnB2, and hEfnB3. In some embodiments, the ephrin ligand mimetic peptide functions as an antagonist to one or more Eph receptors wherein the one or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the ephrin ligand mimetic peptide functions as an antagonist to two or more Eph receptors wherein the two or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the ephrin ligand mimetic peptide functions as an antagonist to EphA2 and EphB2. In some embodiments, the ephrin ligand mimetic peptide functions as an antagonist to three or more Eph receptors wherein the three or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the ephrin ligand mimetic peptide functions as an antagonist to four or more Eph receptors wherein the four or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the ephrin ligand mimetic peptide reduces an extent of phosphorylation of one or more Eph receptors following administering of the composition to a subject. In some embodiments, the phosphorylation of one or more Eph receptors is reduced by at least 10%, 20%, 30%, 40%, 50%, 60%, 70%, 80%, or 90%. In some embodiments, the ephrin ligand mimetic peptide reduces expression of one or more Eph receptors following administering of the composition to a subject. In some embodiments, the expression of EphA2 and EphB2 are reduced following administering of the composition to the subject. In some embodiments, the ephrin ligand mimetic peptide increases regeneration of axonal projections by >500 μm distal to a crush site on a nerve. In some embodiments, the ephrin ligand mimetic peptide induces neurite sprouting following administering of the composition to the subject. In some embodiments, the ephrin ligand mimetic peptide induces local neurite sprouting following administering of the composition locally to the subject. In some embodiments, the ephrin ligand mimetic peptide induces neurite sprouting in retinal ganglion cells (RGCs) following administering of the composition intravitreally to the subject. In some embodiments, the ephrin ligand mimetic peptide protects a plurality of neurons from neurodegeneration following administering of the composition to a subject.

The present disclosure provides methods of treating neurodegenerative disorder in a subject in need thereof. In an aspect, the methods comprise administering to the subject an effective amount of a composition described herein. In some embodiments, the neurodegenerative disorder comprises Alzheimer's disease (AD), traumatic brain injury (TBI), spinal cord injury (SCI), neuropathy, retinopathy, optic neuropathy, glaucoma, glaucomatous degeneration of the optic nerve and retina, age-related macular degeneration (AMD), or stroke. In some embodiments, the neurodegenerative disorder comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, or Hallevorden-Spatz disease. In some embodiments, the neurodegenerative disorder comprises Alzheimer's disease. In some embodiments, the neurodegenerative disorder comprises glaucoma. In some embodiments, the composition is administered systemically or locally. In some embodiments, the composition is administered is administered intravenously, intramuscularly, intrathecally, intracerebrally, subcutaneously, orally, nasally, topically, buccally, or sublingually. In some embodiments, the composition is administered directly to the CNS of the subject via intravenous delivery, intravascular delivery, intrathecal delivery, intracistemal delivery, intraspinal delivery, subpial delivery, or intracerebroventricular delivery. In some embodiments, the composition is administered via stereotaxic injection into the brain parenchyma or the spinal cord parenchyma. In some embodiments, the composition is administered via stereotaxic injection into one or a plurality of regions of cerebral cortex, entorhinal cortex, hippocampus, thalamus, mammillary body, amygdala, or basal ganglia. In some embodiments, the composition is administered to an eye of the subject via intravitreal, subretinal, or retrobulbar injection. In some embodiments, the subject is at a prodromal stage of the neurodegenerative disorder. In some embodiments, an extent of phosphorylation of one or more Eph receptors is reduced following the administering. In some embodiments, the extent of phosphorylation of one or more Eph receptors is reduced by at least 10%, 20%, 30%, 40%, 50%, 60%, 70%, 80%, or 90%. In some embodiments, the extent of phosphorylation of EphA2 and EphB2 is reduced by at least 10%, 20%, 30%, 40%, 50%, 60%, 70%, 80%, or 90%. In some embodiments, the administering prevents a progression of a neurodegenerative disorder in the subject. In some embodiments, the administering provides neuroprotection from a further extent of pathological neuronal cell loss in the subject. In some embodiments, the administering stabilizes a progression of a neurodegenerative disorder in the subject. In some embodiments, the administering improves one or more symptoms of a neurodegenerative disorder in the subject. In some embodiments, the administering increases regeneration of axonal projections by >500 μm distal to a crush site on a nerve.

The present disclosure provides engineered proteins having an amino acid sequence sharing sequence homology with one or more natural ephrin ligands. In an aspect, the engineered proteins comprise an amino acid sequence at least 85% identical to any one of SEQ ID NOs: 1-6. In some embodiments, the amino acid sequence is at least 85% identical to SEQ ID NO: 4, 5, or 6. In some embodiments, the amino acid sequence is 100% identical to SEQ ID NO: 4, 5, or 6. In some embodiments, the engineered protein binds an ephrin receptor.

The present disclosure provides engineered proteins having an amino acid sequence sharing sequence homology with one or more natural ephrin ligands and having various numbers of amino acid substitutions, additions, or deletions. In an aspect, the engineered proteins comprise the amino acid sequence of any one of SEQ ID NOs: 1-6, or a sequence thereof having 1, 2, 3, or 4 amino acid substitutions, additions, or deletions. In some embodiments, the amino acid sequence comprises the amino acid sequence of SEQ ID NO: 4, 5, or 6, or a sequence thereof having 1, 2, 3, or 4 amino acid substitutions, additions, or deletions. In some embodiments, the amino acid sequence comprises the amino acid sequence of SEQ ID NO: 4, 5, or 6, or a sequence thereof having 1 or 2 amino acid substitutions, additions, or deletions. In some embodiments, the amino acid sequence comprises the amino acid sequence of SEQ ID NO: 4, 5, or 6. In some embodiments, the amino acid sequence comprises the amino acid sequence of SEQ ID NO: 4. In some embodiments, the amino acid sequence comprises the amino acid sequence of SEQ ID NO: 5. In some embodiments, wherein the amino acid sequence comprises the amino acid sequence of SEQ ID NO: 6. In some embodiments, an amino acid ASN at an aligned position 1 of SEQ ID NO: 5 interacts with a VAL residue in an EphB2 receptor at an aligned position of VAL94 from SEQ ID NO: 101. In some embodiments, an amino acid ASN at an aligned position 1 of SEQ ID NO: 5 interacts with a VAL residue in an EphB2 receptor at an aligned position of VAL156 from SEQ ID NO: 101. In some embodiments, an amino acid TRP at an aligned position 3 of SEQ ID NO: 5 interacts with a SER residue in an EphB2 receptor at an aligned position of SER47 from SEQ ID NO: 101. In some embodiments, an amino acid GLU at an aligned position 6 of SEQ ID NO: 5 interacts with a ARG residue in an EphB2 receptor at an aligned position of ARG155 from SEQ ID NO: 101. In some embodiments, an amino acid LYS at an aligned position 8 of SEQ ID NO: 5 interacts with a GLU residue in an EphB2 receptor at an aligned position of GLU44 from SEQ ID NO: 101. In some embodiments, an amino acid HIS at an aligned position 11 of SEQ ID NO: 5 interacts with a GLU residue in an EphB2 receptor at an aligned position of GLU44 from SEQ ID NO: 101. In some embodiments, an amino acid TYR at an aligned position 13 of SEQ ID NO: 5 interacts with a GLU residue in an EphB2 receptor at an aligned position of GLU66 from SEQ ID NO: 101. In some embodiments, an amino acid ILE at an aligned position 15 of SEQ ID NO: 5 interacts with a CYS residue in an EphB2 receptor at an aligned position of CYS62 from SEQ ID NO: 101. In some embodiments, an amino acid ILE at an aligned position 15 of SEQ ID NO: 5 interacts with a PHE residue in an EphB2 receptor at an aligned position of PHE65 from SEQ ID NO: 101.

In some embodiments, the engineered protein binds an ephrin receptor. The present disclosure also provides methods of binding an ephrin receptor using an engineered protein described herein. In an aspect, the methods comprise contacting the ephrin receptor with an engineered protein described herein, thereby binding the ephrin receptor. In some embodiments, the ephrin receptor is in a cell. In some embodiments, the ephrin receptor is on a cell. The present disclosure also provides methods of inhibiting activity of an ephrin receptor using an engineered protein described herein. In an aspect, the methods comprise contacting the ephrin receptor with an engineered protein described herein, thereby inhibiting the activity of the ephrin receptor. In some embodiments, the ephrin receptor is in a cell. In some embodiments, the ephrin receptor is on a cell. The present disclosure also provides methods of reducing expression of an ephrin receptor using an engineered protein described herein. In an aspect, the methods comprise contacting a cell with the ephrin receptor with one or more engineered proteins described herein. In some embodiments, the methods comprise contacting a cell with the ephrin receptor with an engineered protein described herein.

The present disclosure provides in vivo modified proteins. In an aspect, the in vivo modified proteins comprise an ephrin receptor bound to a ligand comprising an ephrin ligand mimetic peptide. In some embodiments, the ephrin ligand mimetic peptide is an ephrin ligand mimetic peptide described herein. In some embodiments, the ephrin ligand mimetic peptide comprises an engineered protein described herein.

The present disclosure provides pharmaceutical compositions comprising one or more engineered proteins and a pharmaceutically acceptable carrier. In an aspect, the pharmaceutical compositions comprise one or more engineered proteins described herein and a pharmaceutically acceptable carrier. In some embodiments, the pharmaceutical compositions comprise an engineered protein described herein and a pharmaceutically acceptable carrier. The present disclosure also provides methods of treatment comprising administering an effective amount of a pharmaceutical composition described herein to a subject in need thereof. In some embodiments, the subject has a neurodegenerative disorder. In some embodiments, the subject has a neurodegenerative condition.

The present disclosure provides methods of treating or preventing a neurodegenerative disease in a subject using one or more pan-Eph receptor Ephrin ligand mimetic peptides. In an aspect, the methods comprise administering a therapeutically effective amount of one or more pan-Eph receptor Ephrin ligand mimetic peptides to treat or prevent neurodegenerative disease in the subject. In some embodiments, the neurodegenerative disease is Alzheimer's disease (AD), traumatic brain injury (TBI), spinal cord injury (SCI), neuropathy, retinopathy, optic neuropathy, glaucoma/glaucomatous degeneration of the optic nerve and retina, stroke or other Central Nervous System (CNS) neurodegenerative diseases. In some embodiments, the neurodegenerative disease is AD. In some embodiments, the neurodegenerative disease is glaucoma. In some embodiments, the Ephrin ligand mimetic peptide competitively targets ligand binding sites on the Eph receptor. In some embodiments, the Ephrin ligand mimetic peptide mimics receptor binding domains (RBD) in Ephrin (efn) ligands to compete with the binding of Efn ligands. In some embodiments, the Ephrin ligand mimetic peptide antagonizes the activation of both EphA and EphB class receptors. In some embodiments, the Ephrin ligand mimetic peptide increases regeneration of axonal projections by >500 μm distal to a crush site on a nerve. In some embodiments, the Ephrin ligand mimetic peptide reduces expression of one or more Eph receptors.

The present disclosure provides compositions comprising a pan-Eph receptor Ephrin ligand mimetic peptide. In some embodiments, the Ephrin ligand mimetic peptide competitively targets ligand binding sites on the Eph receptor. In some embodiments, the Ephrin ligand mimetic peptide antagonizes both EphA and EphB receptor activation. In some embodiments, the Ephrin ligand mimetic peptide reduces expression of one or more Eph receptors. In some embodiments, the Ephrin ligand mimetic peptide is selected from the group consisting of SEQ ID NO: 1, SEQ ID NO: 2, SEQ ID NO: 3, SEQ ID NO: 4, SEQ ID NO: 5, or SEQ ID NO: 6.

The present disclosure provides compositions ephrin ligand mimetic peptides at least 15 amino acids in length. In an aspect, the ephrin ligand mimetic peptide comprising an amino acid sequence of ASN-Xaa1-TRP-GLY-Xaa2-GLU-PHE-LYS-Xaa3-Xaa4-HIS-Xaa5-TYR-Xaa6-ILE; wherein Xaa1 is THR, SER TYR, or LEU; wherein Xaa2 is LYS, PHE, TYR, LEU, or HIS; wherein Xaa3 is GLU, PRO, ALA, LYS, or SER; wherein Xaa4 is GLY, HIS, or ASN; wherein Xaa5 is SER GLU, THR, or ASP; and wherein Xaa6 is TYR or PHE. In some embodiments of the ephrin ligand mimetic peptide, an amino acid ASN at an aligned position 1 of SEQ ID NO: 5 interacts with a VAL residue in an EphB2 receptor at an aligned position of VAL94 from SEQ ID NO: 101. In some embodiments of the ephrin ligand mimetic peptide, an amino acid ASN at an aligned position 1 of SEQ ID NO: 5 interacts with a VAL residue in an EphB2 receptor at an aligned position of VAL156 from SEQ ID NO: 101. In some embodiments of the ephrin ligand mimetic peptide, an amino acid TRP at an aligned position 3 of SEQ ID NO: 5 interacts with a SER residue in an EphB2 receptor at an aligned position of SER47 from SEQ ID NO: 101. In some embodiments of the ephrin ligand mimetic peptide, an amino acid GLU at an aligned position 6 of SEQ ID NO: 5 interacts with a ARG residue in an EphB2 receptor at an aligned position of ARG155 from SEQ ID NO: 101. In some embodiments of the ephrin ligand mimetic peptide, an amino acid LYS at an aligned position 8 of SEQ ID NO: 5 interacts with a GLU residue in an EphB2 receptor at an aligned position of GLU44 from SEQ ID NO: 101. In some embodiments of the ephrin ligand mimetic peptide, an amino acid HIS at an aligned position 11 of SEQ ID NO: 5 interacts with a GLU residue in an EphB2 receptor at an aligned position of GLU44 from SEQ ID NO: 101. In some embodiments of the ephrin ligand mimetic peptide, an amino acid TYR at an aligned position 13 of SEQ ID NO: 5 interacts with a GLU residue in an EphB2 receptor at an aligned position of GLU66 from SEQ ID NO: 101. In some embodiments of the ephrin ligand mimetic peptide, an amino acid ILE at an aligned position 15 of SEQ ID NO: 5 interacts with a CYS residue in an EphB2 receptor at an aligned position of CYS62 from SEQ ID NO: 10. In some embodiments of the ephrin ligand mimetic peptide, an amino acid ILE at an aligned position 15 of SEQ ID NO: 5 interacts with a PHE residue in an EphB2 receptor at an aligned position of PHE65 from SEQ ID NO: 101. In some embodiments, the ephrin ligand mimetic peptide forms an in vivo bound protein complex by binding to one or more ephrin receptors. In some embodiments, the ephrin ligand mimetic peptide antagonizes both EphA and EphB receptor activation. In some embodiments, the ephrin ligand mimetic peptide mimics one or more receptor binding domains (RBD) in one or more ephrin ligands to compete with the binding of natural ephrin ligands. In some embodiments, the ephrin ligand mimetic peptide functions as an antagonist to EphA2, EphB2 and EphB3. In some embodiments, the ephrin ligand mimetic peptide functions as an antagonist to EphA2 and EphB2. In some embodiments, the ephrin ligand mimetic peptide reduces an extent of phosphorylation of one or more Eph receptors following administering of the composition to a subject. In some embodiments, the ephrin ligand mimetic peptide reduces expression of one or more Eph receptors following administering of the composition to a subject. In some embodiments, the expression of EphA2 and EphB2 are reduced following administering of the composition to the subject. In some embodiments, the ephrin ligand mimetic peptide increases regeneration of axonal projections by >500 μm distal to a crush site on a nerve. In some embodiments, the ephrin ligand mimetic peptide induces neurite sprouting following administering of the composition to the subject. In some embodiments, the ephrin ligand mimetic peptide protects a plurality of neurons from neurodegeneration following administering of the composition to a subject.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “certain embodiment,” “some embodiments,” “various embodiments,” “related embodiments,” each of these types of embodiments is a non-limiting example of a feature that is intended to be combined with any other feature, or combination of features, described herein without having to list every possible combination. Such features or combinations of features apply to any of the aspects of the invention.

The headings herein are for the convenience of the reader and not intended to be limiting. Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIGS. 1A-1C: Ephrin ligand sequence homology and in-silico peptide docking models. (FIG. 1A) Amino acid sequence alignment for human Ephrin (Efn) ligands for hEfn-A (A1, A2, A3, A4, A5) and hEfn-B (B1, B2 and B3). Sequences obtained from the National Center for Biotechnology Information (NCBI). Receptor binding domain (RBD) motif homology is underlined for Efn-A and Efn-B subclasses. Alignment of RBD motifs from hEfn-A1-A5 and hEfn-B1-B3 are shown relative to xEFN peptides: RBD, RBD1, RBD2, RBD3, and RBD4. (FIG. 1B) Peptide (xEFN)-Protein (Eph-R) docking models for xEFN_RBD2 peptide with EPHA4 (upper left) and EPHB2 (upper right); xEFN_RBD3 peptide with EPHA4 (lower left) and EPHB2 (lower right). (FIG. 1C) Peptide-Protein docking models showing the top three docking conformations and model estimated energies of each for peptide xEFN_RBD4 and EphA4 (top), or EphB2 (bottom) receptors.

FIGS. 2A-2B: The effect of xEFN peptides, RBD2 and RBD3, on neurite outgrowth from isolated mouse RGCs in vitro over short term (48 h, FIG. 2A) and long term (6 days, FIG. 2B) growth.

FIGS. 3A-3F: The effect of xEFN peptide supplementation on Eph-receptor modulation in in vitro assays on isolated mouse RGCs. (FIG. 3A) ELISA assays following supplementation of culture media with 500 nM xEFN_RBD1-4 for 4 days. (FIG. 3B) Immunohistochemistry of TUBB3 and pEphA2 receptor in RGCs supplemented with 500 nM xEFN_RBD3 or control for 4 days. (FIG. 3C) Western blot analysis of RhoA, pRac1, Ephexin-5 and pEphexin protein levels (versus Beta-actin control) following treatment of RBCs with 500 nM xEFN_RBD1, xEFN_RBD2, xEFN_RBD3 or xEFN_RBD4. (FIG. 3D) Analysis of growth cone dynamics following supplementation of media with 500 nM xEFN_RBD3 for 4 days using mouse RGC cultures. Effect after 4 days of 1p M xEFN peptide treatment of isolated mouse RGCs in-vitro on the (FIG. 3E) expression of EphA2 and EphB2 receptors by western blot and (FIG. 3F) morphological presentation of Beta-3 tubulin (TUBB3) and filamentous actin (F-actin) by immunofluorescent staining. Treatment with xEFN_RBD3 and xEFN_RBD4 effectively decreased expression of EphA2 and EphB2 as shown in FIG. 3E. xEFN_RBD2 did not appear to have as strong of an effect in altering expression of EphA2 or EphB2. Treatment with xEFN_RBD2, xEFN_RBD3, and xEFN_RBD4 effectively preserved RGCs in this assay as seen by a high density and overall area of staining of TUBB3 thereby demonstrating a role for xEFN_RBD2, xEFN_RBD3, and xEFN_RBD4 in neuroprotection of retinal neurons.

FIGS. 4A-4D: Assessing the role of xEFN in antagonizing this repulsive stimulus on human hippocampal neural stem cells (hNSC). (FIG. 4A) Laminin-coated surfaces containing pre-clustered efnA1, A2, A4, B1, or B3 (FIG. 4B-4D) Hippocampal neural stem cells grown on these substrates achieve confluences of 51.16+/−1.55% for efnA4, 48.39+/−3.38% for efnA2, and 44.61+/−1.61% for efnB3 when compared to the laminin-only control confluence.

FIGS. 5A-5D: Pan-Eph receptor antagonization leads to significant RGC neuroprotection and axonal regeneration in an animal model of traumatic optic neuropathy (TON). (FIG. 5A) Immunohistochemistry images and (FIG. 5B) quantification of RGCs in TUBB3-labeled retinal flatmounts. (FIG. 5C) Fluorescent microscopy images of dissected optic tract of animal cohorts, and (FIG. 5D) Spectrofluorometric quantification of axonal projections per optic nerve in the 2 mm nerve segment proximal to the crush site. (**p<0.01).

FIGS. 6A-6C: Upregulation of Eph receptors in early neuropathic diseases of the visual system. (FIG. 6A) Phospho-proteomic analysis of Eph receptor activation in DBA/2J at different ages. (FIG. 6B) IHC of EphB1 presentation in the RGC layer. (FIG. 6C) IHC of activated EphB1+B2 in DBA/2J at different ages.

FIGS. 7A-7B: Genetic knockdown of Eph receptors via AAV2-mediated shRNA transduction. (FIG. 7A) Amino acid and nucleotide targeting sequences for the shEPHR constructs. (FIG. 7B) In vitro fluorescent imaging of cultured primary mouse RGCs and the neurite networks following transduction with shCNTRL or shEPHR constructs, western blot of EphB1 and EphA4 showing knockdown of protein with shEPHR.

FIGS. 8A-8C: Multiparametric assessment of visual system morphology and function.

FIG. 9: Table of shRNA constructs targeting transcripts for Ephrin ligands and Eph receptors.

FIGS. 10A-10C: (FIG. 10A) Table of conserved residues and motifs within the ephrin ligand receptor binding domain. (FIG. 10B) Table of sequences for final xEFN peptide library. (FIG. 10C) Table of antibodies used and specifications.

FIG. 11A-11D: Crystalized structures of ephrin ligand Eph-receptor interactions showing promiscuity and cross-class interactions. 2WO2 (Bowden T A 2009) EphA4-ephrinB2 (FIG. 11A); 2WO3 (Bowden T A 2009) EphA4-ephrinA2 (FIG. 11B); 1SHW (Himanen J P 2004) EphB2-ephrinA5 (FIG. 11C); 1KGY (Himanen J P 2001) EphB2-ephrinB2 (FIG. 11D).

FIGS. 12A-12C: Amino acid sequence alignment for ephrin ligands between mouse (Mus musculus) and human (Homo sapiens) orthologs (FIG. 12A), and alignment and homology of xEFN peptide targeting region between the two species (FIGS. 12B and 12C).

FIG. 13: Representative image of the real-time, label-free neurite segmentation and quantitation performed by the Incucyte Zoom NeuroTrack™ Processing algorithm used in the study.

FIGS. 14A-14B: (FIG. 14A) In silico residue substitution analysis on the xEFN-F1 homologous efn RDB region showing critical amino acid residues conferring cross-class promiscuity as well as affinity of the RBD sequence to the respective receptor. (FIG. 14B) Diagrams of conformational docking and calculated model energies with given amino acid substitutions analyzed.

FIG. 15: Table of xEFN peptides identified by in-silico modeling of the xEFN_RBD2 peptide sequence. The 15 xEFN peptide sequences shown have higher binding affinities to EphA4 and/or EphB2 compared with the xEFN_RBD2 peptide sequence.

FIG. 16: Schematic of experimental timeline. Time-course RNA-Seq after onset of ON. RNA-Sequencing of retina s/p SI-TON over time (n=9) to elucidate the dynamics of the injury signature. Characterize the cascade of molecular events that precipitate the neuropathic progression. Identify targeted therapies. Identify synergistic and/or combinatorial neuroprotective treatments.

FIG. 17: Schematic of experimental timeline. Ephrin ligand mimetic peptides to modulate Eph-receptor signaling after the onset of clinically-relevant optic neuropathy (ON).

FIG. 18: Ephrin receptor antagonism with xEFN2 enhances RGC survival in ON. Representative confocal images of βIII tubulin (TUBB3) labeled retinal ganglion cells (immunofluorescently labeled in the first three rows) and CTB labeled axon bundles (immunofluorescently labeled in the last row) in flat-mounted retinas 28 days after SI-TON injury.

FIGS. 19A-19C: Administration of xEFN_RBD2 and xEFN_RBD3 peptides in a clinically relevant animal model of optic neuropathy. (FIG. 19A) Schematic of experimental timeline. (FIG. 19B) Immunohistochemistry images of beta 3 tubulin in excised retinas 4 weeks after the onset of optic neuropathy. (FIG. 19C) Quantification of RGC survival in all animal groups relative to naïve (uninjured) retinal RGC counts.

FIGS. 20A-20B: Sequential pattern electroretinogram (PERG) recordings at baseline, 2 weeks, and 4 weeks post optic neuropathy onset. (FIG. 20A) Box and whiskers plot of PERG response amplitudes at baseline, 2-weeks, and 4-weeks post injury. (FIG. 20B) Individual animal PERG waveform traces at baseline, 2-weeks, and 4-weeks post injury.

FIGS. 21A-21B: Pattern electroretinogram (PERG) measurement 2 weeks post optic neuropathy onset. (FIG. 21A) Box and whiskers plot of PERG response amplitudes for each individual animal at baseline and 2-weeks post injury. (FIG. 21B) Representative PERG waveforms in the vehicle (PBS) and xEFN_RBD2 and xEFN_RBD3 treatment groups at baseline and 2 weeks post injury.

FIGS. 22A-22B: Flash electroretinogram (ERG) recordings to assess outer retinal function 2-weeks post optic neuropathy onset. (FIG. 22A) B-wave response amplitude in dark adapted (DA) animals at 0.01 cm·s/m² to assess rod photoreceptor function. (FIG. 22B) B-wave response amplitude in dark adapted (DA) animals at 1.0 cm·s/m² to assess combined rod and cone photoreceptor function.

FIGS. 23A-23D: Binding kinetics as measured by label-free Biolayer Interferometry (BLI) of a published EphB2 targeting peptide sequence (SNEW) as well as xEFN peptides to the (FIG. 23A) EphA2, (FIG. 23B) EphB2, and (FIG. 23C) EphB3 receptors. (FIG. 23D) calculated dissociation constant (KD) for the different peptide-receptor interactions measured.

FIGS. 24A-24C: xEFN_RBD3 molecular interaction with EphB2 and competition with ephrinB1. (FIG. 24A) shows a docking model generated using the GLIDE docking software suite between xEFN_RBD3 and a resolved crystal structure for EphB2. (FIG. 24B) is a diagram showing key molecular interactions between xEFN_RBD3 and the EphB2 ligand binding pocket (LBP). (FIG. 24C) shows results graphed of an in vitro binding assay displaying percent binding of EphB2 to ephrinB1 in the presence of an increasing concentration of xEFN_RBD3 demonstrating a competitive antagonist action of xEFN_RBD3 in this molecular interaction.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims.

The skilled artisan will recognize many variants and adaptations of the aspects described herein. These variants and adaptations are intended to be included in the teachings of this disclosure and to be encompassed by the claims herein.

Eph/efn signaling is critical for proper development of the central nervous system (CNS) (6-12). Eph-receptor activation has been associated with the onset of several neurodegenerative diseases of the CNS including Alzheimer's disease (AD) (48-54), glaucomatous degeneration of the optic nerve and retina, traumatic brain injury (TBI), stroke, and spinal cord injury (SCI), among others. However, there are many technical challenges to targeting Eph-receptor activation in the CNS that have not been adequately addressed by any approach explored so far. Most studies have focused on one or two class-specific receptor modulation. Similarly, and as highlighted in AD and glaucoma, several individual Eph-receptors are implicated in each CNS neurodegenerative disease, with no one receptor demonstrating a dominant role in neurodegenerative onset or progression. While great strides have been made into Eph receptor targeting with small molecules and peptides, most developed compounds are designed for specificity to a particular Eph-receptor. As such, there is a significant need to develop effective therapies to prevent or treat symptoms associated with neurodegenerative diseases which involve several Eph receptors' activity as part of their pathophysiology.

One such strategy is described herein. The disclosure provides compositions and methods of treating or preventing a neurodegenerative disease in a subject comprising administering one or more pan-Eph receptor Ephrin ligand mimetic peptides in an amount effective to treat or prevent neurodegenerative disease in the subject.

Some aspects relate to a peptide or protein. The peptide or protein may bind to a receptor tyrosine kinase (RTK) such as an Ephrin receptor. The binding may inhibit or decrease expression of the receptor tyrosine kinase (e.g., an Ephrin receptor). The peptide or protein may be an Ephrin ligand mimetic. The peptide or protein may be synthetic. The peptide or protein may be recombinant. The peptide or protein may be engineered. The peptide or protein may be included in a composition or method disclosed herein.

### Ephrin Receptors (Eph) and Ephrin Ligands (Efn)

Ephrin receptors (Eph) and ephrin ligands (efn) constitute the largest family of receptor tyrosine kinases (RTK) in mammalian biology. Eph/efn members consist of at least 16 types of Eph-receptors discovered thus far (10 EphA, and 6 EphB receptors). Both Eph-receptors and ephrin ligands are membrane-bound proteins that transduce intracellular signaling when activated.

To date, studies looking into individual Eph/ephrin moieties in retinal neurodegenerative diseases have identified EphA4, EphA5, EphB1, EphB2, and EphB3 as well as several individual ephrin ligands as being associated with RGC loss, neuroinflammation, and neurodegenerative processes.

### Pan- or Multi-Eph Receptor Targeting Peptide

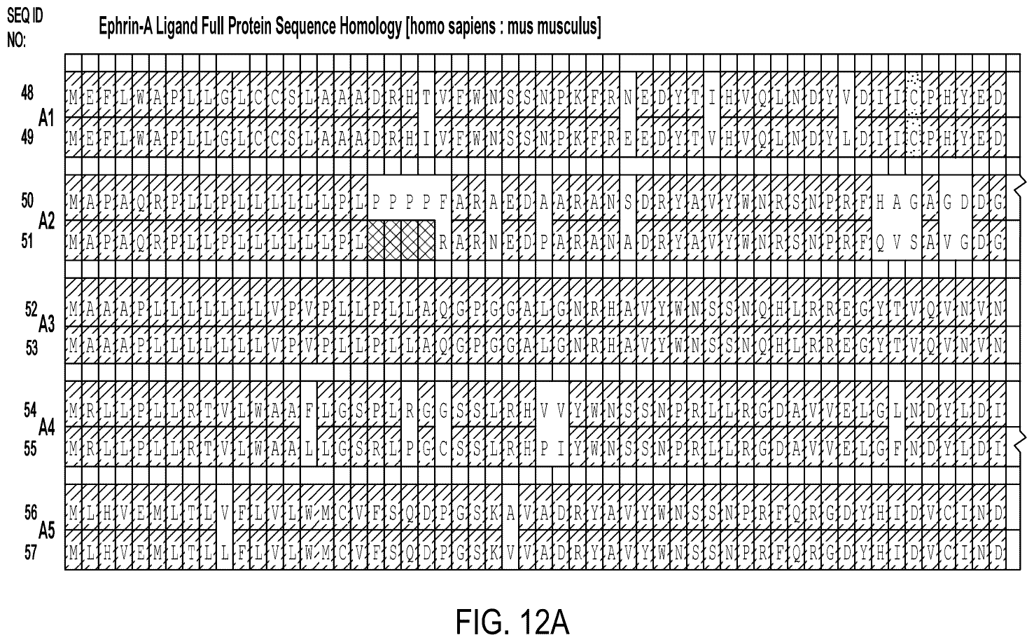

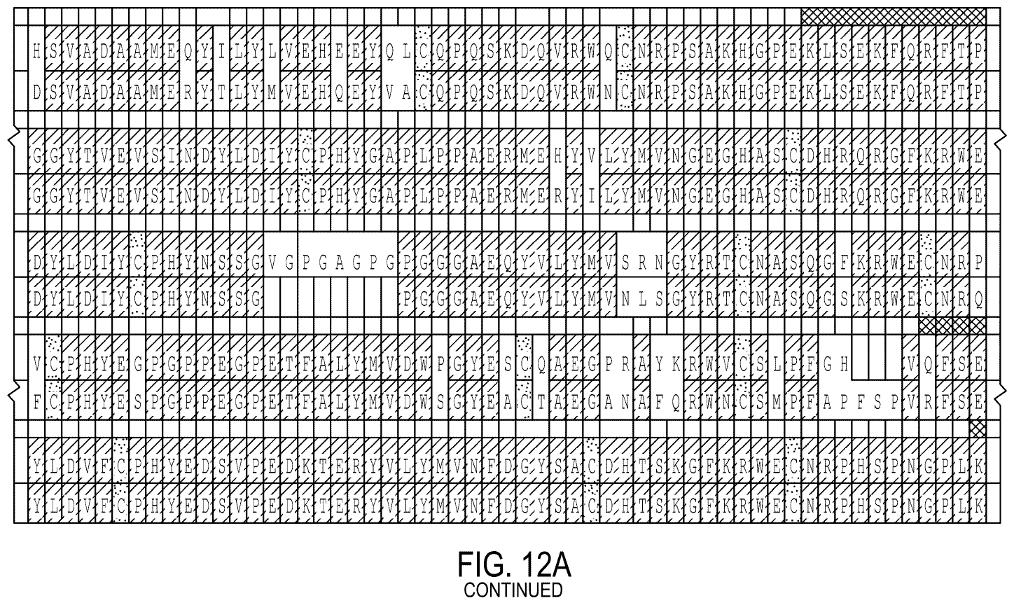

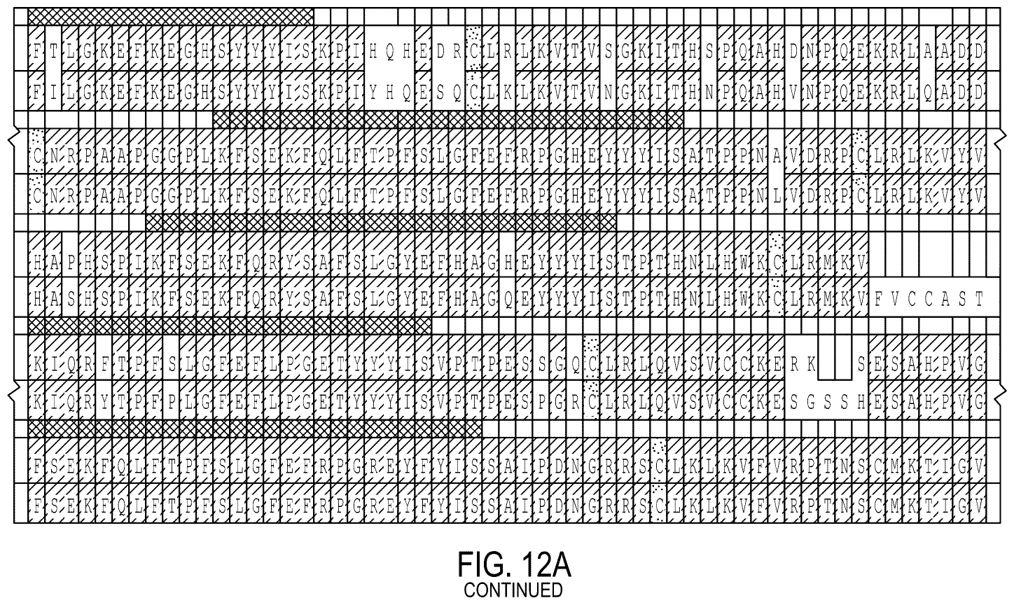

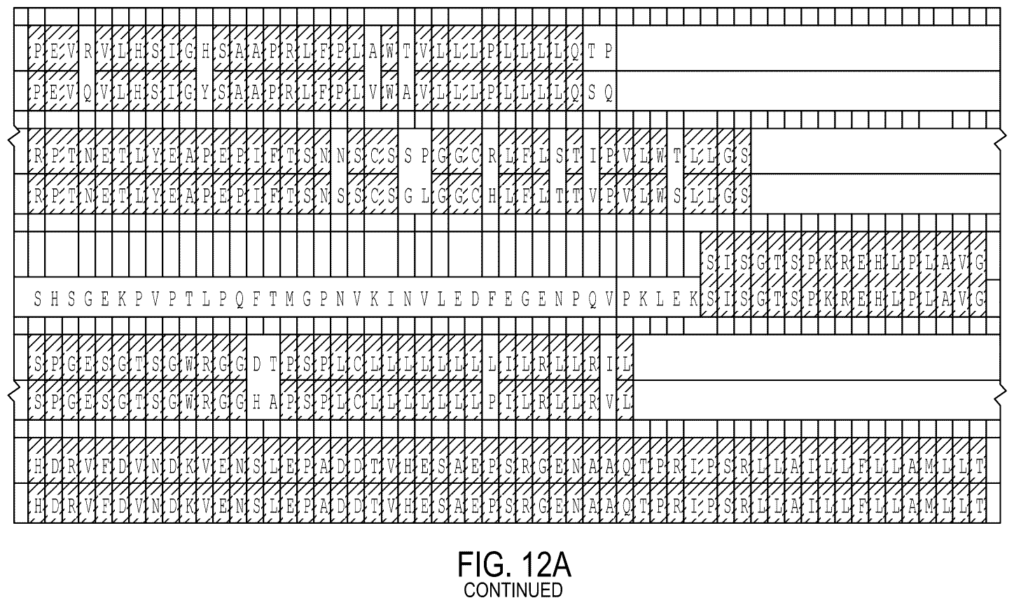

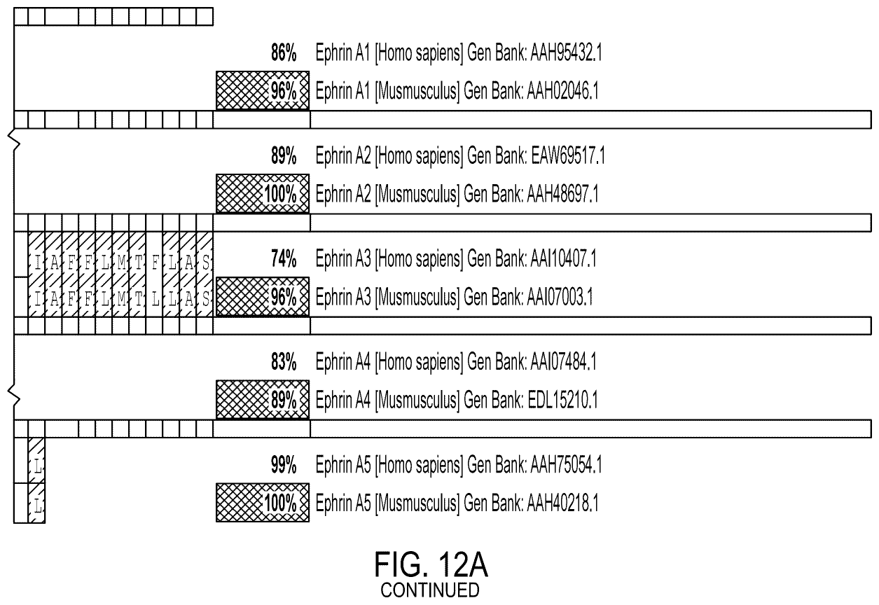

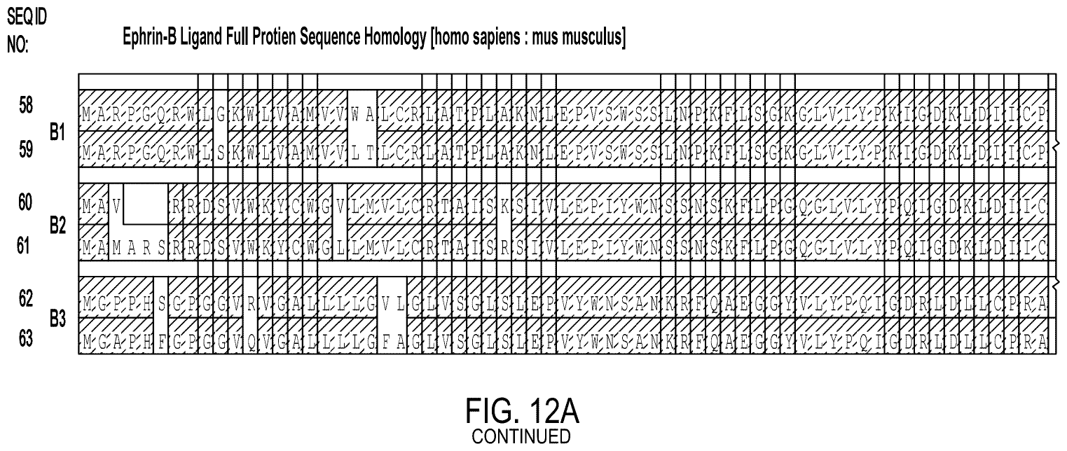

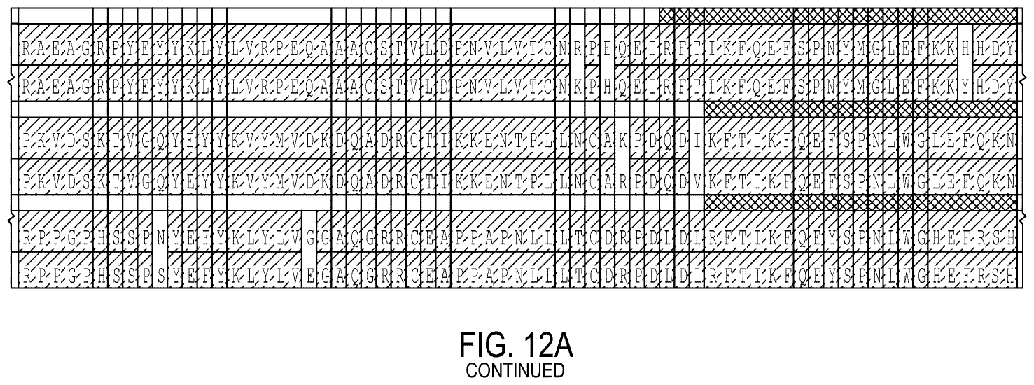

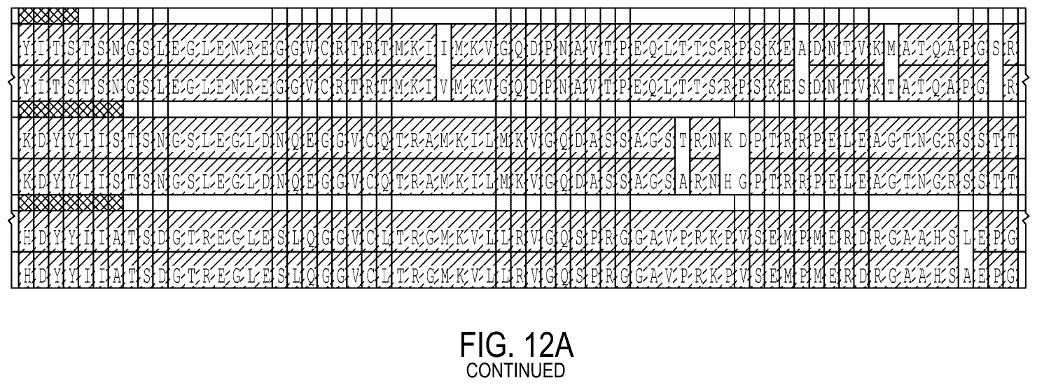

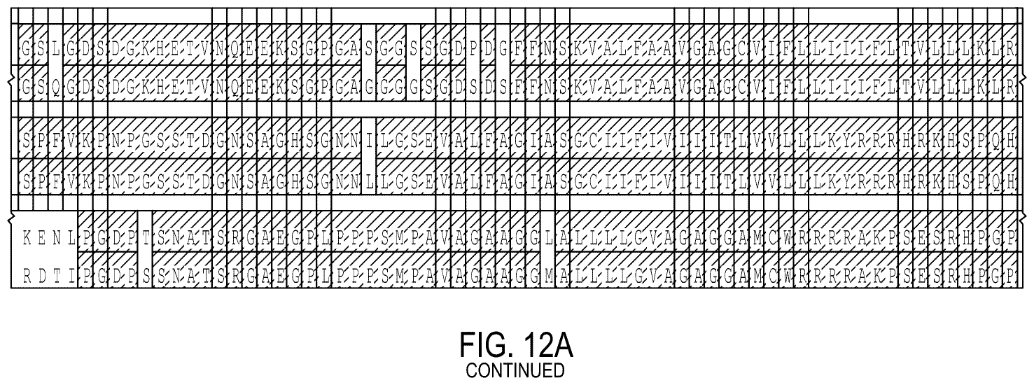

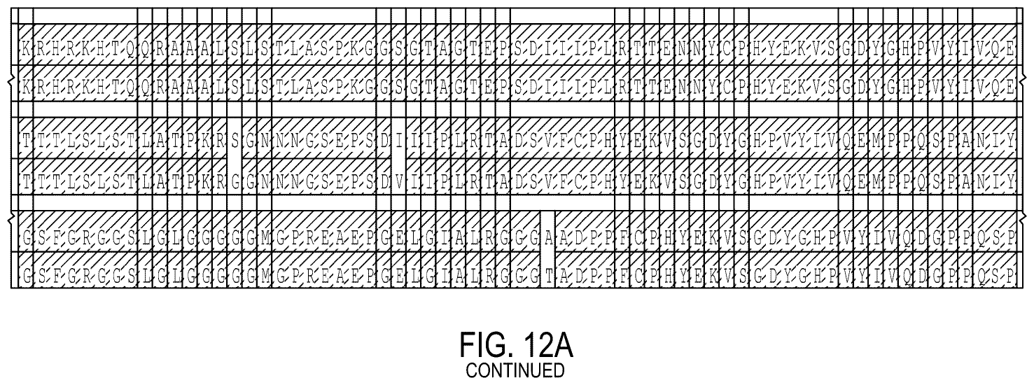

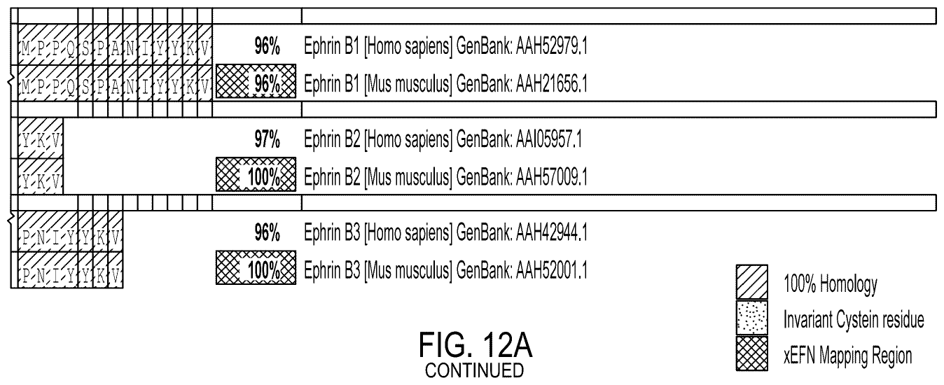

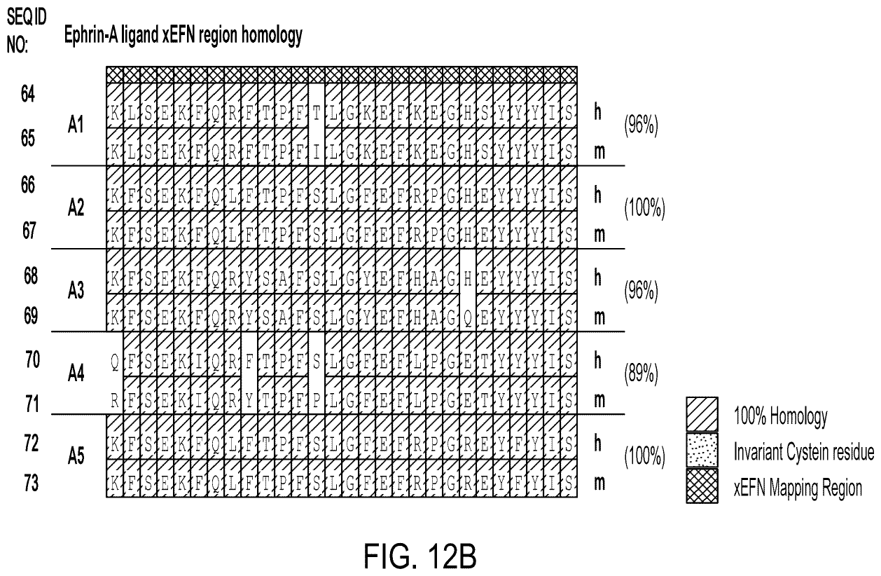

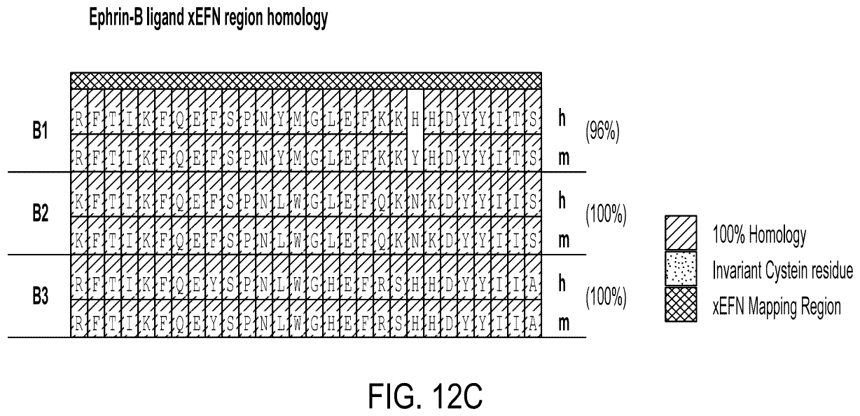

Disclosed herein are peptide species capable of modulating Eph-receptor signaling in a pan-receptor manner. By targeting the homology of receptor binding domains (RBD) in efn ligands, and the known interactions that allow for promiscuity in cross-class ligand-receptor interactions, an efn RBD mimetic peptide library was designed for screening and identifying lead peptides (xEFN) for in vivo testing. The sequence homology is so highly conserved across mammalian species, that human and mouse efn sequences are almost perfectly identical in their receptor binding domain (RBD) (FIGS. 12A-12C), allowing for the design of human mimetic species that retain biological activity in the mouse CNS. In order to achieve this, in-silico alignment and binding analyses of ephrin-ligand RBD motifs were performed for all ephrin ligands, and homologous sequences were identified to create peptide libraries with predicted antagonistic biological activity to multi-Eph-receptor activation for in vitro and in vivo testing.

Disclosed herein is data on peptide molecules, xEFN-RBD2, xEFN-RBD3, and xEFN-RBD4 (collectively xEFN).

As disclosed herein, the treatment of isolated mouse retinal ganglion cells (RGC) or human fetal hippocampal neural stem cells (hNSC) with xEFN peptides produces a significant increase in neurite sprouting, axonal extension, and desensitization of neuronal process to growth on repulsive efn-bound surfaces. Treatment of neurons with xEFN peptides was shown to antagonize phosphorylation of Eph receptors and downstream activators. In an optic nerve crush model (ONC), two intravitreal administrations of xEFN peptides led to significant neuroprotection of RGCs and axonal bundle morphology and resulted in significant regeneration of axonal projections along the optic nerve when compared to vehicle-treated controls at 4-weeks post crush. These data demonstrated that modulation of multiple Eph-receptors by xEFN peptides leads to significant neuronal rescue and regeneration in neurodegenerative processes of the CNS.

In various aspects, the disclosure provides compositions and methods wherein the Ephrin ligand mimetic peptide mimics the receptor binding domains (RBD) in Ephrin (efn) ligands. In related aspects, the Ephrin ligand mimetic peptide antagonizes both EphAs and EphBs receptors. In related aspects, the Ephrin ligand mimetic peptide antagonizes EphAs or EphBs receptors. In related aspects, the Ephrin ligand mimetic peptide increases regeneration of axonal projections by >500 μm distal to a crush site on a nerve. In various aspects, the Ephrin ligand mimetic peptide is a pan-Eph receptor Ephrin ligand mimetic peptide. In related aspects, the Ephrin ligand mimetic peptide sequence is SEQ ID NO: 1, SEQ ID NO: 2, SEQ ID NO: 3, SEQ ID NO: 4, SEQ ID NO: 5, or SEQ ID NO: 6. In some embodiments, the ephrin ligand mimetic peptide is a peptide ligand. In some embodiments, the peptide ligand binds to an Ephrin receptor. In some embodiments, the peptide ligand binds to one or more Ephrin receptors. In some embodiments, the peptide ligand is bound to a Ephrin receptor forming an in vivo modified protein. In some embodiments, an in vivo modified protein comprises an Ephrin receptor bound to a peptide ligand. In some embodiments, the peptide ligand shares amino acid sequence homology with one or more ephrin ligands. In some embodiments, the peptide ligand shares amino acid sequence identity with one or more ephrin ligands. In some embodiments, the peptide ligand shares amino acid sequence alignment with one or more ephrin ligands.

The method of the disclosure “blocks” or “antagonizes” phosphorylation of one or more Eph receptors. “Antagonize” is used herein to refer to any reduction in the phosphorylation and display of pEph receptor expression in cells or tissue of a subject (e.g., 20% reduction, 30% reduction, 40% reduction, 50% reduction, 60% reduction, 70% reduction, 80% reduction, or 90% reduction). “Antagonizing” phosphorylation of one or more Eph receptors does not require 100% abolition of pEph receptor expression, although the disclosure contemplates 100% inhibition of pEph receptor expression in various aspects.

### Compositions

Described herein are compositions comprising an ephrin ligand mimetic peptide. In some embodiments, the compositions comprise one or more ephrin ligand mimetic peptides. In some embodiments, the compositions comprise two or more ephrin ligand mimetic peptides. Also described herein are compositions comprising a pan-Eph receptor Ephrin ligand mimetic peptide. In some embodiments, the compositions comprise one or more a pan-Eph receptor Ephrin ligand mimetic peptides. In some embodiments, the compositions comprise two or more a pan-Eph receptor Ephrin ligand mimetic peptides.

Also described herein are compositions comprising ephrin ligand mimetic peptides comprising an amino acid sequence at least 15 amino acid residues in length. Compositions described herein can be used in methods of treating one or more neurodegenerative disorders. Compositions described herein can be used in methods of treating one or more neurodegenerative diseases. Compositions described herein can be used in methods of treating one or more neurodegenerative conditions. Compositions described herein can be used in methods of preventing one or more neurodegenerative disorders. Compositions described herein can be used in methods of preventing one or more neurodegenerative conditions. In some embodiments, the composition described herein is provided for preventing, inhibiting the progress of, treating, or ameliorating one or more symptoms of Alzheimer's disease in a subject. In some embodiments, a composition described herein is provided for preventing, inhibiting the progress of, treating, or ameliorating one or more symptoms of TBI in a subject. In some embodiments, a composition described herein is provided for preventing, inhibiting the progress of, treating, or ameliorating one or more symptoms of an optic neuropathy in a subject. In some embodiments, the optical neuropathy comprises glaucoma. In some embodiments, a composition described herein is provided for preventing, inhibiting the progress of, treating, or ameliorating one or more symptoms of glaucoma in a subject. In some embodiments, a composition described herein is provided for preventing, inhibiting the progress of, treating, or ameliorating one or more symptoms of a spinal cord injury (SCI) in a subject. In some embodiments, a composition described herein is provided for preventing inhibiting the progress of, treating, or ameliorating one or more symptoms of a neuropathy in a subject. In some embodiments, a composition described herein is provided for preventing, inhibiting the progress of, treating, or ameliorating one or more symptoms of a retinopathy in a subject. In some embodiments, a composition described herein is provided for preventing, inhibiting the progress of, treating, or ameliorating one or more symptoms of glaucomatous degeneration of the optic nerve and retina in a subject. In some embodiments, a composition described herein is provided for preventing, inhibiting the progress of, treating, or ameliorating one or more symptoms of a retinopathy in a subject. In some embodiments, a composition described herein is provided for preventing, inhibiting the progress of, treating, or ameliorating one or more symptoms of age-related macular degeneration (AMD) in a subject. In some embodiments, a composition described herein is provided for preventing, inhibiting the progress of, treating, or ameliorating one or more symptoms of stroke in a subject. Provided herein, in certain aspects, are compositions that prevent or inhibit the progress of a tauopathy in a subject. The composition may include an ephrin ligand binding protein. The composition may include an ephrin ligand. The composition may include a pan- or multi-Eph receptor targeting peptide.

In some embodiments, the compositions described herein inhibit the progression of Alzheimer's disease in a subject. In some embodiments, the compositions described herein prevent the progression of Alzheimer's disease in a subject. In some embodiments, the compositions described herein prevent or inhibit the progression of Alzheimer's disease in a subject. Alzheimer's disease typically progresses in three stages—early (mild), middle (moderate), and late (severe). In the early (mild) stage of Alzheimer's disease, patients may experience difficulty performing complex tasks, may lose or misplace belongings, or have difficulty organizing or planning. In the middle (moderate) stage of Alzheimer's disease, patients may experience memory loss of events or personal history, depression, having trouble controlling their bladder and bowels, wandering and getting lost, or exhibit personality changes. In the late (severe) stage of Alzheimer's disease, patients may experience difficulty in communicating or inability to walk without assistance. In advanced stages of AD, the progression of the disease can be fatal. In some embodiments, the compositions described herein prevent or inhibit the progress of early-stage Alzheimer's disease in a subject. In some embodiments, the compositions described herein prevent or inhibit the progress of middle-stage Alzheimer's disease in a subject. In some embodiments, the compositions described herein prevent or inhibit the progress of late-stage Alzheimer's disease in a subject.

In some embodiments, the compositions described herein prevent the progress of a neurodegenerative disorder in the subject. In some embodiments, the compositions described herein inhibit the progress of a neurodegenerative disorder in the subject. In some embodiments, the compositions described herein prevent or inhibit the progress of a neurodegenerative disorder in the subject. In some embodiments, the compositions described herein prevent or inhibit the progress of a neurodegenerative disease in the subject. In some embodiments, the compositions described herein prevent or inhibit the progress of a neurodegenerative condition in the subject. In some embodiments, the compositions described herein prevent or inhibit the progress of AD in a subject. In some embodiments, the compositions described herein prevent or inhibit the progress of glaucoma in a subject. In some embodiments, the compositions described herein inhibit the progress of TBI in a subject. In some embodiments, the compositions described herein prevent or inhibit the progress of a retinopathy in a subject. In some embodiments, the compositions described herein prevent or inhibit the progress of glaucomatous degeneration of the optic nerve and retina in a subject. In some embodiments, the compositions described herein prevent or inhibit the progress of a neuropathy in a subject. In some embodiments, the compositions described herein prevent or inhibit the progress of an optic neuropathy in a subject. In some embodiments, the compositions described herein inhibit the progress of an SCI in a subject.

In some embodiments, the compositions described herein prevent or inhibit the progress of Niemann-Pick disease type C in a subject. In some embodiments, the compositions described herein prevent or inhibit the progress of Frontal temporal dementia (FTD) in a subject. In some embodiments, the compositions described herein prevent or inhibit the progress of frontotemporal lobar degeneration in a subject. In some embodiments, the compositions described herein prevent or inhibit the progress of chronic traumatic encephalopathy (CTE) in a subject. In some embodiments, the compositions described herein prevent or inhibit the progress of progressive supranuclear palsy (PSP) in a subject. In some embodiments, the compositions described herein prevent or inhibit the progress of corticobasal degeneration (CBD) in a subject. In some embodiments, the compositions described herein prevent or inhibit the progress of Lytico-Bodig disease in a subject. In some embodiments, the compositions described herein prevent or inhibit the progress of tangle-predominant dementia in a subject. In some embodiments, the compositions described herein prevent or inhibit the progress of primary age-related tauopathy (PART) in a subject. In some embodiments, the compositions described herein prevent or inhibit the progress of argyrophilic grain disease (AGD) in a subject. In some embodiments, the compositions described herein prevent or inhibit the progress of globular glial tauopathy (GGT) in a subject. In some embodiments, the compositions described herein prevent or inhibit the progress of vacuolar tauopathy in a subject. In some embodiments, the compositions described herein prevent or inhibit the progress of tuberous sclerosis in a subject. In some embodiments, the compositions described herein prevent or inhibit the progress of postencephalitic parkinsonism in a subject. In some embodiments, the compositions described herein prevent or inhibit the progress of subacute sclerosing panencephalitis in a subject. In some embodiments, the compositions described herein prevent or inhibit the progress of amyotrophic lateral sclerosis in a subject. In some embodiments, the compositions described herein prevent or inhibit the progress of myotonic dystrophy in a subject. In some embodiments, the compositions described herein prevent or inhibit the progress of Pallido-ponto-nigral degeneration in a subject. In some embodiments, the compositions described herein prevent or inhibit the progress of Parkinson's disease in a subject. In some embodiments, the compositions described herein prevent or inhibit the progress of Creutzfeldt-Jacob disease in a subject. In some embodiments, the compositions described herein prevent or inhibit the progress of Dementia pugilistica in a subject. In some embodiments, the compositions described herein prevent or inhibit the progress of Down's syndrome in a subject. In some embodiments, the compositions described herein prevent or inhibit the progress of Gerstmann-Staussler-Scheinker disease in a subject. In some embodiments, the compositions described herein prevent or inhibit the progress of inclusion-body myositis in a subject. In some embodiments, the compositions described herein prevent or inhibit the progress of diffuse neurofibrillary tangles with calcification in a subject. In some embodiments, the compositions described herein prevent or inhibit the progress of Tangle-only dementia in a subject. In some embodiments, the compositions described herein prevent or inhibit the progress of Hallevorden-Spatz disease in a subject.

In some embodiments, the compositions described herein treat or ameliorate one or more symptoms of a neurodegenerative disease in a subject. In some embodiments, the compositions described herein treat or ameliorate one or more symptoms of AD in a subject. In some embodiments, the compositions described herein treat or ameliorate one or more symptoms of a TBI in a subject. In some embodiments, the compositions described herein treat or ameliorate one or more symptoms of glaucoma in a subject. In some embodiments, the compositions described herein treat or ameliorate one or more symptoms of glaucomatous degeneration of the optic nerve and retina in a subject. In some embodiments, the compositions described herein treat or ameliorate one or more symptoms of TBI in a subject. In some embodiments, the compositions described herein treat or ameliorate one or more symptoms of SCI in a subject. In some embodiments, the compositions described herein treat or ameliorate one or more symptoms of a neuropathy in a subject. In some embodiments, the compositions described herein treat or ameliorate one or more symptoms of a retinopathy in a subject. In some embodiments, the compositions described herein treat or ameliorate one or more symptoms of an optic neuropathy in a subject. In some embodiments, the compositions described herein treat or ameliorate one or more symptoms of AMD in a subject. In some embodiments, the compositions described herein treat or ameliorate one or more symptoms of stroke in a subject. In some embodiments, the compositions described herein treat or ameliorate one or more symptoms of a tauopathy in a subject. In some embodiments, the compositions described herein treat or ameliorate one or more symptoms of Pick's disease in a subject. In some embodiments, the compositions described herein treat or ameliorate one or more symptoms of Niemann-Pick disease type C in a subject. In some embodiments, the compositions described herein treat or ameliorate one or more symptoms of Frontal temporal dementia (FTD) in a subject. In some embodiments, the compositions described herein treat or ameliorate one or more symptoms of frontotemporal lobar degeneration in a subject. In some embodiments, the compositions described herein treat or ameliorate one or more symptoms of chronic traumatic encephalopathy (CTE) in a subject. In some embodiments, the compositions described herein treat or ameliorate one or more symptoms of progressive supranuclear palsy (PSP) in a subject. In some embodiments, the compositions described herein treat or ameliorate one or more symptoms of corticobasal degeneration (CBD) in a subject. In some embodiments, the compositions described herein treat or ameliorate one or more symptoms of Lytico-Bodig disease in a subject. In some embodiments, the compositions described herein treat or ameliorate one or more symptoms of tangle-predominant dementia in a subject. In some embodiments, the compositions described herein treat or ameliorate one or more symptoms of meningioaniomatosis in a subject. In some embodiments, the compositions described herein treat or ameliorate one or more symptoms of primary age-related tauopathy (PART) in a subject. In some embodiments, the compositions described herein treat or ameliorate one or more symptoms of argyrophilic grain disease (AGD) in a subject. In some embodiments, the compositions described herein treat or ameliorate one or more symptoms of globular glial tauopathy (GGT) in a subject. In some embodiments, the compositions described herein treat or ameliorate one or more symptoms of vacuolar tauopathy in a subject. In some embodiments, the pharmaceutical compositions described herein treat or ameliorate one or more symptoms of tuberous sclerosis in a subject. In some embodiments, the compositions described herein treat or ameliorate one or more symptoms of postencephalitic parkinsonism in a subject. In some embodiments, the compositions described herein treat or ameliorate one or more symptoms of amyotrophic lateral sclerosis in a subject. In some embodiments, the compositions described herein treat or ameliorate one or more symptoms of myotonic dystrophy in a subject. In some embodiments, the compositions described herein treat or ameliorate one or more symptoms of Pallido-ponto-nigral degeneration in a subject. In some embodiments, the compositions described herein treat or ameliorate one or more symptoms of Parkinson's disease in a subject. In some embodiments, the compositions described herein treat or ameliorate one or more symptoms of Creutzfeldt-Jacob disease in a subject. In some embodiments, the compositions described herein treat or ameliorate one or more symptoms of Dementia pugilistica in a subject. In some embodiments, the compositions described herein treat or ameliorate one or more symptoms of Down's syndrome in a subject. In some embodiments, the compositions described herein treat or ameliorate one or more symptoms of Gerstmann-Staussler-Scheinker disease in a subject. In some embodiments, the compositions described herein treat or ameliorate one or more symptoms of inclusion-body myositis in a subject. In some embodiments, the compositions described herein treat or ameliorate one or more symptoms of diffuse neurofibrillary tangles with calcification in a subject. In some embodiments, the compositions described herein treat or ameliorate one or more symptoms of Tangle-only dementia in a subject. In some embodiments, the compositions described herein treat or ameliorate one or more symptoms of Hallevorden-Spatz disease in a subject.

In an aspect, compositions described herein comprise a ephrin ligand mimetic peptide. In some embodiments, the ephrin ligand mimetic peptide comprising an amino acid sequence having a particular sequence identity to one or more ephrin ligands. In some embodiments, the ephrin ligand mimetic peptide comprising an amino acid sequence having a particular sequence homology to one or more ephrin ligands. In some embodiments, the ephrin ligand mimetic peptide comprising an amino acid sequence having a particular sequence alignment to one or more ephrin ligands. In some embodiments, the region of a particular sequence identity is highly conserved among ephrin ligand family members. In some embodiments, the region of a particular sequence identity is highly conserved among ephrin A ligand family members. In some embodiments, the region of a particular sequence identity is highly conserved among two, three, four, or five of EfnA1, EfnA2, EfnA3, EfnA4, and EfnA5.

In some embodiments, the region of a particular sequence identity is highly conserved among two of EfnA1, EfnA2, EfnA3, EfnA4, and EfnA5. In some embodiments, the region of a particular sequence identity is highly conserved among three of EfnA1, EfnA2, EfnA3, EfnA4, and EfnA5. In some embodiments, the region of a particular sequence identity is highly conserved among four of EfnA1, EfnA2, EfnA3, EfnA4, and EfnA5. In some embodiments, the region of a particular sequence identity is highly conserved among all five of EfnA1, EfnA2, EfnA3, EfnA4, and EfnA5. In some embodiments, the region of a particular sequence identity is highly conserved among ephrin B ligand family members. In some embodiments, the region of a particular sequence identity is highly conserved among two or three of EfnB1, EfnB2, and EfnB3. In some embodiments, the region of a particular sequence identity is highly conserved among two of EfnB1, EfnB2, EfnB3. In some embodiments, the region of a particular sequence identity is highly conserved among two, three, four, five, or more of EfnA1, EfnA2, EfnA3, EfnA4, EfnA5, EfnB1, EfnB2, and EfnB3. In some embodiments, the region of a particular sequence identity is highly conserved among two of EfnA1, EfnA2, EfnA3, EfnA4, EfnA5, EfnB1, EfnB2, and EfnB3. In some embodiments, the region of a particular sequence identity is highly conserved among three of EfnA1, EfnA2, EfnA3, EfnA4, EfnA5, EfnB1, EfnB2, and EfnB3. In some embodiments, the region of a particular sequence identity is highly conserved among four of EfnA1, EfnA2, EfnA3, EfnA4, EfnA5, EfnB1, EfnB2, and EfnB3. In some embodiments, the region of a particular sequence identity is highly conserved among five of EfnA1, EfnA2, EfnA3, EfnA4, EfnA5, EfnB1, EfnB2, and EfnB3. In some embodiments, the region of a particular sequence identity is highly conserved among six of EfnA1, EfnA2, EfnA3, EfnA4, EfnA5, EfnB1, EfnB2, and EfnB3. In some embodiments, the region of a particular sequence identity is highly conserved among seven of EfnA1, EfnA2, EfnA3, EfnA4, EfnA5, EfnB1, EfnB2, and EfnB3. In some embodiments, the region of a particular sequence identity is highly conserved among all eight of EfnA1, EfnA2, EfnA3, EfnA4, EfnA5, EfnB1, EfnB2, and EfnB3. In some embodiments, the particular sequence identity between the ephrin ligand mimetic peptide and any one of EfnA1, EfnA2, EfnA3, EfnA4, EfnA5, EfnB1, EfnB2, or EfnB3 is at least about 70%, 75%, 80%, 85%, 90%, 95%, 97%, or is 100% identical. In some embodiments, the particular sequence identity between the ephrin ligand mimetic peptide and any one of EfnA1, EfnA2, EfnA3, EfnA4, EfnA5, EfnB1, EfnB2, or EfnB3 is at least about 85% identical. In some embodiments, the particular sequence identity between the ephrin ligand mimetic peptide and any one of EfnA1, EfnA2, EfnA3, EfnA4, EfnA5, EfnB1, EfnB2, or EfnB3 is at least about 90% identical. In some embodiments, the particular sequence identity between the ephrin ligand mimetic peptide and any one of EfnA1, EfnA2, EfnA3, EfnA4, EfnA5, EfnB1, EfnB2, or EfnB3 is at least about 95% identical. In some embodiments, the particular sequence identity between the ephrin ligand mimetic peptide and any one of EfnA1, EfnA2, EfnA3, EfnA4, EfnA5, EfnB1, EfnB2, or EfnB3 is 100% identical. In some embodiments, the ephrin ligand mimetic peptide comprises an amino acid sequence at least 90% identical to a sequence selected from SEQ ID NOs: 1-6. In some embodiments, the ephrin ligand mimetic peptide comprises an amino acid sequence at least 90% identical to a sequence selected from SEQ ID NOs: 1-2, or 4-6. In some embodiments, the ephrin ligand mimetic peptide comprises an amino acid sequence at least 90% identical to a sequence selected from SEQ ID NOs: 4-6. In some embodiments, the ephrin ligand mimetic peptide comprises an amino acid sequence at least 90% identical to SEQ ID NO: 1. In some embodiments, the ephrin ligand mimetic peptide comprises an amino acid sequence at least 90% identical to SEQ ID NO: 2. In some embodiments, the ephrin ligand mimetic peptide comprises an amino acid sequence at least 90% identical to SEQ ID NO: 3. In some embodiments, the ephrin ligand mimetic peptide comprises an amino acid sequence at least 90% identical to SEQ ID NO: 4. In some embodiments, the ephrin ligand mimetic peptide comprises an amino acid sequence at least 90% identical to SEQ ID NO: 5. In some embodiments, the ephrin ligand mimetic peptide comprises an amino acid sequence at least 90% identical to SEQ ID NO: 6. In some embodiments, the ephrin ligand mimetic peptide comprises an amino acid sequence at least 95% identical to a sequence selected from SEQ ID NOs: 1-6. In some embodiments, the ephrin ligand mimetic peptide comprises an amino acid sequence at least 95% identical to a sequence selected from SEQ ID NOs: 1-2, or 4-6. In some embodiments, the ephrin ligand mimetic peptide comprises an amino acid sequence at least 95% identical to a sequence selected from SEQ ID NOs: 4-6. In some embodiments, the ephrin ligand mimetic peptide comprises an amino acid sequence at least 95% identical to SEQ ID NO: 1. In some embodiments, the ephrin ligand mimetic peptide comprises an amino acid sequence at least 95% identical to SEQ ID NO: 2. In some embodiments, the ephrin ligand mimetic peptide comprises an amino acid sequence at least 95% identical to SEQ ID NO: 3. In some embodiments, the ephrin ligand mimetic peptide comprises an amino acid sequence at least 95% identical to SEQ ID NO: 4. In some embodiments, the ephrin ligand mimetic peptide comprises an amino acid sequence at least 95% identical to SEQ ID NO: 5. In some embodiments, the ephrin ligand mimetic peptide comprises an amino acid sequence at least 95% identical to SEQ ID NO: 6. In some embodiments, the ephrin ligand mimetic peptide comprises an amino acid sequence selected from SEQ ID NOs: 1-6. In some embodiments, the ephrin ligand mimetic peptide comprises an amino acid sequence selected from SEQ ID NOs: 1-2, or 4-6. In some embodiments, the ephrin ligand mimetic peptide comprises an amino acid sequence selected from SEQ ID NOs: 4-6. In some embodiments, the ephrin ligand mimetic peptide comprises an amino acid sequence selected from SEQ ID NOs: 5-6. In some embodiments, the ephrin ligand mimetic peptide comprises an amino acid sequence from SEQ ID NO: 1. In some embodiments, the ephrin ligand mimetic peptide comprises an amino acid sequence from SEQ ID NO: 2. In some embodiments, the ephrin ligand mimetic peptide comprises an amino acid sequence from SEQ ID NO: 3. In some embodiments, the ephrin ligand mimetic peptide comprises an amino acid sequence from SEQ ID NO: 4. In some embodiments, the ephrin ligand mimetic peptide comprises an amino acid sequence from SEQ ID NO: 5. In some embodiments, the ephrin ligand mimetic peptide comprises an amino acid sequence from SEQ ID NO: 6. In some embodiments, the ephrin ligand mimetic peptide comprises an amino acid sequence from any one of SEQ ID NO: 17-25. In some embodiments, the ephrin ligand mimetic peptide comprises an amino acid sequence from any one of SEQ ID NO: 64-99. In some embodiments, the ephrin ligand mimetic peptide comprises an amino acid sequence designated by SEQ ID NO: 7. SEQ ID NO: 7 is designated as Phe-Xaa-Xaa-Lys-Phe-Gln-Xaa-Phe-Xaa-Pro-Xaa-Xaa-Xaa-Phe-Xaa-Xaa-Phe-Xaa-Xaa-Xaa-Xaa-Xaa-Tyr-Tyr-Xaa-Ile-Xaa; wherein miscellaneous feature (MISC-FEAT) 1 and 2 are any naturally occurring amino acid, MISC-FEAT 3 is Leu or Glu, MISC-FEAT 4 is Ser or Thr, MISC-FEAT 5 is Phe or Asn, MISC-FEAT 5 is Ser or Leu, MISC-FEAT 6 is Leu, MISC-FEAT 7 is any naturally occurring amino acid, MISC-FEAT 8 is Glu or Ala, MISC-FEAT 9-13 are any naturally occurring amino acid, MISC-FEAT 14 is Ile or Tyr, and MISC-FEAT 15 is any naturally occurring amino acid.

In some embodiments, the ephrin ligand mimetic peptide comprises an amino acid sequence sharing identity with a sequence from one or more of EfnA1, EfnA2, EfnA3, EfnA4, EfnA5, EfnB1, EfnB2, or EfnB3 and has one amino acid addition, one amino acid deletion, or one or two amino acid substitutions. In some embodiments, the ephrin ligand mimetic peptide comprises an amino acid sequence having one amino acid addition from a sequence selected from SEQ ID NOs: 1-6. In some embodiments, the ephrin ligand mimetic peptide comprises an amino acid sequence having one amino acid deletion from a sequence selected from SEQ ID NOs: 1-6. In some embodiments, the ephrin ligand mimetic peptide comprises an amino acid sequence having one or two amino acid substitutions from a sequence selected from SEQ ID NOs: 1-6. In some embodiments, the ephrin ligand mimetic peptide comprises an amino acid sequence having one or two amino acid substitutions from a sequence selected from SEQ ID NOs: 1-2, or 4-6. In some embodiments, the ephrin ligand mimetic peptide comprises an amino acid sequence having one amino acid addition from a sequence selected from SEQ ID NOs: 1-2, or 4-6. In some embodiments, the ephrin ligand mimetic peptide comprises an amino acid sequence having one amino acid deletion from a sequence selected from SEQ ID NOs: 1-2, or 4-6. In some embodiments, the ephrin ligand mimetic peptide comprises an amino acid sequence having one amino acid addition from a sequence selected from SEQ ID NOs: 4-6. In some embodiments, the ephrin ligand mimetic peptide comprises an amino acid sequence having one amino acid deletion from a sequence selected from SEQ ID NOs: 4-6. In some embodiments, the ephrin ligand mimetic peptide comprises an amino acid sequence having one amino acid addition, deletion, or substitution from SEQ ID NO: 1. In some embodiments, the ephrin ligand mimetic peptide comprises an amino acid sequence having one amino acid addition, deletion, or substitution from SEQ ID NO: 2. In some embodiments, the ephrin ligand mimetic peptide comprises an amino acid sequence having one amino acid addition, deletion, or substitution from SEQ ID NO: 3. In some embodiments, the ephrin ligand mimetic peptide comprises an amino acid sequence having one amino acid addition, deletion, or substitution from SEQ ID NO: 4. In some embodiments, the ephrin ligand mimetic peptide comprises an amino acid sequence having one amino acid addition, deletion, or substitution from SEQ ID NO: 5. In some embodiments, the ephrin ligand mimetic peptide comprises an amino acid sequence having one amino acid addition, deletion, or substitution from SEQ ID NO: 6. In some embodiments, the ephrin ligand mimetic peptide comprises an amino acid sequence having one amino acid addition from SEQ ID NO: 1. In some embodiments, the ephrin ligand mimetic peptide comprises an amino acid sequence having one amino acid addition from SEQ ID NO: 2. In some embodiments, the ephrin ligand mimetic peptide comprises an amino acid sequence having one amino acid addition from SEQ ID NO: 3. In some embodiments, the ephrin ligand mimetic peptide comprises an amino acid sequence having one amino acid addition from SEQ ID NO: 4. In some embodiments, the ephrin ligand mimetic peptide comprises an amino acid sequence having one amino acid addition from SEQ ID NO: 5. In some embodiments, the ephrin ligand mimetic peptide comprises an amino acid sequence having one amino acid addition from SEQ ID NO: 6. In some embodiments, the ephrin ligand mimetic peptide comprises an amino acid sequence having one amino acid deletion from SEQ ID NO: 1. In some embodiments, the ephrin ligand mimetic peptide comprises an amino acid sequence having one amino acid deletion from SEQ ID NO: 2. In some embodiments, the ephrin ligand mimetic peptide comprises an amino acid sequence having one amino acid deletion from SEQ ID NO: 3. In some embodiments, the ephrin ligand mimetic peptide comprises an amino acid sequence having one amino acid deletion from SEQ ID NO: 4. In some embodiments, the ephrin ligand mimetic peptide comprises an amino acid sequence having one amino acid deletion from SEQ ID NO: 5. In some embodiments, the ephrin ligand mimetic peptide comprises an amino acid sequence having one amino acid deletion from SEQ ID NO: 6. In some embodiments, the ephrin ligand mimetic peptide comprises an amino acid sequence having one amino acid substitution from SEQ ID NO: 1. In some embodiments, the ephrin ligand mimetic peptide comprises an amino acid sequence having one amino acid substitution from SEQ ID NO: 2. In some embodiments, the ephrin ligand mimetic peptide comprises an amino acid sequence having one amino acid substitution from SEQ ID NO: 3. In some embodiments, the ephrin ligand mimetic peptide comprises an amino acid sequence having one amino acid substitution from SEQ ID NO: 4. In some embodiments, the ephrin ligand mimetic peptide comprises an amino acid sequence having one amino acid substitution from SEQ ID NO: 5. In some embodiments, the ephrin ligand mimetic peptide comprises an amino acid sequence having one amino acid substitution from SEQ ID NO: 6. In some embodiments, the one or two amino acid substitutions may be conservative amino acid substitutions. In some embodiments, the amino acid substitution may be a conservative amino acid substitution.

In some embodiments, the composition comprises an ephrin ligand mimetic peptide. In some embodiments, the composition comprises an ephrin ligand mimetic peptide that consists essentially of an amino acid sequence selected from SEQ ID NOs: 1-6. In some embodiments, the composition comprises an ephrin ligand mimetic peptide that consists essentially of an amino acid sequence selected from SEQ ID NOs: 1-2, or 4-6. In some embodiments, the composition comprises an ephrin ligand mimetic peptide that consists essentially of an amino acid sequence selected from SEQ ID NOs: 4-6. In some embodiments, the composition comprises an ephrin ligand mimetic peptide that consists essentially of an amino acid sequence of SEQ ID NO: 1. In some embodiments, the composition comprises an ephrin ligand mimetic peptide that consists essentially of an amino acid sequence of SEQ ID NO: 2. In some embodiments, the composition comprises an ephrin ligand mimetic peptide that consists essentially of an amino acid sequence of SEQ ID NO: 3. In some embodiments, the composition comprises an ephrin ligand mimetic peptide that consists essentially of an amino acid sequence of SEQ ID NO: 4. In some embodiments, the composition comprises an ephrin ligand mimetic peptide that consists essentially of an amino acid sequence of SEQ ID NO: 5. In some embodiments, the composition comprises an ephrin ligand mimetic peptide that consists essentially of an amino acid sequence of SEQ ID NO: 6. In some embodiments, the composition consists essential of an ephrin ligand mimetic peptide and a one or more pharmaceutically acceptable carriers. In some embodiments, the composition consists essential of an ephrin ligand mimetic peptide and one or more pharmaceutically acceptable excipients. In some embodiments, the composition consists essential of an ephrin ligand mimetic peptide having an amino acid sequence selected from SEQ ID NOs: 1-6 and a one or more pharmaceutically acceptable carriers. In some embodiments, the composition consists essential of an ephrin ligand mimetic peptide having an amino acid sequence selected from SEQ ID NOs: 1-6 and a one or more pharmaceutically acceptable excipients. In some embodiments, the composition consists essential of an ephrin ligand mimetic peptide having an amino acid sequence of SEQ ID NO: 1 and a pharmaceutically acceptable carrier. In some embodiments, the composition consists essential of an ephrin ligand mimetic peptide having an amino acid sequence of SEQ ID NO: 2 and a pharmaceutically acceptable carrier. In some embodiments, the composition consists essential of an ephrin ligand mimetic peptide having an amino acid sequence of SEQ ID NO: 3 and a pharmaceutically acceptable carrier. In some embodiments, the composition consists essential of an ephrin ligand mimetic peptide having an amino acid sequence of SEQ ID NO: 4 and a pharmaceutically acceptable carrier. In some embodiments, the composition consists essential of an ephrin ligand mimetic peptide having an amino acid sequence of SEQ ID NO: 5 and a pharmaceutically acceptable carrier. In some embodiments, the composition consists essential of an ephrin ligand mimetic peptide having an amino acid sequence of SEQ ID NO: 6 and a pharmaceutically acceptable carrier. In some embodiments, one or more amino acid substitutions in a ephrin ligand mimetic peptide described here are conservative substitutions in which the substituted amino acid(s) share similar characteristics. In some embodiments, one or more amino acid substitutions are made between amino acids listed in the same class in Table 1. A non-limiting example of a conservative amino acid substitution would be between Leu and Ile as they are both aliphatic, branched hydrophobes. Another non-limiting example of a conservative amino acid substitution would be between Asp and Glu as they are both small, negatively charged residues.

TABLE 1
Amino Acid Classes and Conservative Substitutions

| Class | Amino Acid | Three Letter Code |
|---|---|---|
| Aliphatic | Glycine, Alanine, Valine, Leucine, Isoleucine | Gly, Ala, Val, Leu, Ile |
| Hydroxyl or | Serine, Cysteine, Threonine, Methionine | Ser, Cys, Thr, Met |
| sulfur- | | |
| containing | | |
| Cyclic | Proline | Pro |
| Aromatic | Phenylalanine, Tyrosine, Tryptophan | Phe, Tyr, Trp |
| Basic | Histidine, Lysine, Arginine | His, Lys, Arg |
| Acidic and | Aspartic Acid, Glutamic Acid, Asparagine, | Asp, Glu, Asn, Gln |
| their amides | Glutamine | |

In some embodiments, the composition comprising the ephrin ligand mimetic peptide elicits a biological response upon contacting one or more cells. In some embodiments, the ephrin ligand mimetic peptide elicits a biological response following administering the composition to the subject. In some embodiments, the ephrin ligand mimetic peptide elicits a biological response upon contacting one or more cells following administering in a subject. In some embodiments, the biological response is in vitro. In some embodiments, the biological response is ex vivo. In some embodiments, the biological response is in vivo. In some embodiments, the ephrin ligand mimetic peptide targets a ligand binding site on one or more Eph receptors. In some embodiments, the ephrin ligand mimetic peptide targets a plurality of ligand binding sites on one or more Eph receptors. In some embodiments, the ephrin ligand mimetic peptide targets at least two ligand binding sites on one or more Eph receptors. In some embodiments, the ephrin ligand mimetic peptide targets at least three ligand binding sites on one or more Eph receptors. In some embodiments, the ephrin ligand mimetic peptide targets at least four ligand binding sites on one or more Eph receptors. In some embodiments, the ephrin ligand mimetic peptide targets at least five ligand binding sites on one or more Eph receptors. In some embodiments, the ephrin ligand mimetic peptide targets at least six ligand binding sites on one or more Eph receptors. In some embodiments, the ephrin ligand mimetic peptide targets at least seven ligand binding sites on one or more Eph receptors. In some embodiments, the ephrin ligand mimetic peptide targets at least eight ligand binding sites on one or more Eph receptors. In some embodiments, the ephrin ligand mimetic peptide competitively targets one or more ligand binding sites on one or more Eph receptors. In some embodiments, competitive targeting comprises ephrin ligand mimetic peptide binding to one or more Eph receptors with a higher binding affinity than a natural ephrin ligand. In some embodiments, the natural ephrin ligand is an ephrin-A ligand and wherein the one or more Eph receptors is selected from the group consisting of EphA1, EphA2, EphA3, EphA4, EphA5, EphA6, EphA7, EphA8, and EphA10. In some embodiments, the natural ephrin ligand is an ephrin-A ligand and wherein the ephrin-A ligand is ephrin-AT, ephrin-A2, ephrin-A3, ephrin-A4, or ephrin-A5. In some embodiments, the natural ephrin ligand is an ephrin-A ligand and wherein the ephrin-A ligand is human ephrin-A1, human ephrin-A2, human ephrin-A3, human ephrin-A4, or human ephrin-A5. In some embodiments, the natural ephrin ligand is an ephrin-B ligand and wherein the one or more Eph receptors is selected from the group consisting of EphB1, EphB2, EphB3, EphB4, and EphB6. In some embodiments, the natural ephrin ligand is an ephrin-B ligand and wherein the ephrin-B ligand is ephrin-B1, ephrin-B2, or ephrin-B3. In some embodiments, the natural ephrin ligand is an ephrin-B ligand and the ephrin-B ligand is human ephrin-B1, human ephrin-B2, or human ephrin-B3.

In some embodiments, the ephrin ligand mimetic peptide binds to one or more Eph receptors at various amino acid sites within the extracellular domain of the Eph receptor. In some embodiments, the ephrin ligand mimetic peptide binds to EphA1. In some embodiments, the ephrin ligand mimetic peptide binds to EphA2. In some embodiments, the ephrin ligand mimetic peptide binds to EphA3. In some embodiments, the ephrin ligand mimetic peptide binds to EphA4. In some embodiments, the ephrin ligand mimetic peptide binds to EphA5. In some embodiments, the ephrin ligand mimetic peptide binds to EphA6. In some embodiments, the ephrin ligand mimetic peptide binds to EphA7. In some embodiments, the ephrin ligand mimetic peptide binds to EphA8. In some embodiments, the ephrin ligand mimetic peptide binds to EphA10. In some embodiments, the ephrin ligand mimetic peptide binds to EphB1. In some embodiments, the ephrin ligand mimetic peptide binds to EphB2. In some embodiments, the ephrin ligand mimetic peptide binds to EphB3. In some embodiments, the ephrin ligand mimetic peptide binds to EphB4. In some embodiments, the ephrin ligand mimetic peptide binds to EphB6. In some embodiments, the ephrin ligand mimetic peptide binds to EphB2 at any of GLU44, SER47, CYS62, PHE65, GLU66, VAL94, ARG155, or VAL156 in relation to SEQ ID NO: 101.

In some embodiments, the ephrin ligand mimetic peptide mimics one or more receptor binding domains (RBD) in one or more ephrin ligands to compete with the binding of natural ephrin ligands. In some embodiments, the ephrin ligand mimetic peptide mimics one or more receptor binding domains (RBD) found in hEfnA1, hEfnA2, hEfnA3, hEfnA4, hEfnA5, hEfnB1, hEfnB2, or hEfnB3. In some embodiments, the ephrin ligand mimetic peptide mimics two or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics three or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics four or more receptor binding domains (RBD) found in the group consisting of hEfnA1, hEfnA2, hEfnA3, hEfnA4, hEfnA5, hEfnB1, hEfnB2, and hEfnB3. In some embodiments, the ephrin ligand mimetic peptide mimics receptor binding domains (RBD) found in hEfnA1, hEfnA2, hEfnA3, hEfnA4, hEfnA5, hEfnB1, hEfnB2, and hEfnB3.

In some embodiments, the composition comprising an ephrin ligand mimetic peptide regulates a functional property of one or more Eph receptors. In some embodiments, the functional property is regulated following administering of the composition to the subject. In some embodiments, the functional property is receptor signaling. In some embodiments, receptor signaling is antagonized. In some embodiments, the functional property is Eph receptor phosphorylation. In some embodiments, phosphorylation of one or more Eph receptors is reduced. In some embodiments, the functional property is Eph receptor expression. In some embodiment, protein expression of one or more Eph receptors is reduced. In some embodiments, the ephrin ligand mimetic peptide functions as an antagonist to one or more Eph receptors. In some embodiments, the ephrin ligand mimetic peptide is a peptide ligand. In some embodiments, the peptide ligand binds to one or more of Eph receptors. In some embodiments, the peptide ligand binds to at least two Eph receptors. In some embodiments, the peptide ligand functions as an antagonist to one or more Eph receptors. In some embodiments, the peptide ligand functions as an antagonist to one or more Eph receptors wherein the one or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the peptide ligand functions as an antagonist to two or more Eph receptors wherein the two or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the peptide ligand functions as an antagonist to EphA2 and EphB2. In some embodiments, the peptide ligand functions as an antagonist to hEphA2 and hEphB2. In some embodiments, the peptide ligand functions as an antagonist to EphA2, EphB2, and EphB3. In some embodiments, the peptide ligand functions as an antagonist to hEphA2, hEphB2, and hEphB3. In some embodiments, the peptide ligand functions as an antagonist to any one receptor of EphA2, EphB2, or EphB3. In some embodiments, the peptide ligand functions as an antagonist to three or more Eph receptors wherein the three or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the peptide ligand functions as an antagonist to four or more Eph receptors wherein the four or more Eph receptors are selected from EphA1, EphA2, EphA3, EphA4, EphA5, EphA6, EphA7, EphA8, EphA10, EphB1, EphB2, EphB3, EphB4, and EphB6. In some embodiments, the peptide ligand reduces an extent of phosphorylation of one or more Eph receptors following administering of the composition to a subject. In some embodiments, the peptide ligand reduces an extent of phosphorylation of one or more Eph receptors is reduced by at least 10%, 20%, 30%, 40%, 50%, 60%, 70%, 80%, or 90%. In some embodiments, the peptide ligand reduces an extent of phosphorylation of one or more Eph receptors is reduced by at least 20%. In some embodiments, the peptide ligand reduces an extent of phosphorylation of one or more Eph receptors is reduced by at least 50%. In some embodiments, the peptide ligand reduces an extent of phosphorylation of one or more Eph receptors is reduced by at least 70%. In some embodiments, the peptide ligand reduces an extent of phosphorylation of one or more Eph receptors is reduced by at least 90%. In some embodiments, the peptide ligand reduces expression of one or more Eph receptors following administering of the composition to a subject. In some embodiments, the expression of EphA2 and EphB2 are reduced following administering of the composition to the subject.

In some embodiments, the composition comprising an ephrin ligand mimetic peptide regulates a cellular behavior in one or more neurons. In some embodiments, the ephrin ligand mimetic peptide is a peptide ligand. In some embodiments, the peptide ligand binds to one or more of Eph receptors. In some embodiments, the cellular behavior in one or more neurons is regulated following administering of the composition to the subject. In some embodiments, the cellular behavior in one or more neurons is regulated following administering of a therapeutically effective amount of the composition to the subject. In some embodiments, the peptide ligand increases regeneration of axonal projections by >500 μm distal to a crush site on a nerve. In some embodiments, the peptide ligand induces neurite sprouting following administering of the composition to the subject. In some embodiments, the peptide ligand induces local neurite sprouting following administering of the composition locally to the subject. In some embodiments, the peptide ligand induces neurite sprouting in retinal ganglion cells (RGCs) following administering of the composition intravitreally to the subject. In some embodiments, the peptide ligand protects a plurality of neurons from neurodegeneration following administering of the composition to a subject. In some embodiments, the peptide ligand protects a plurality of neurons from neurodegeneration in a subject having a neurodegenerative condition following administering of the composition to the subject.

In an aspect described herein are engineered proteins sharing a degree of sequence identity with one or more ephrin proteins. In some embodiments, the engineered protein is a peptide ligand. In some embodiments, the peptide ligand can bind to one or more Eph receptors. In some embodiments, the peptide ligand bound to an Eph receptor forms an in vivo modified protein following administering of a composition comprising an engineered protein. In some embodiments, the engineered protein comprises an amino acid sequence at least 85% identical to any one of SEQ ID NOs: 1-6. In some embodiments, the engineered protein comprises an amino acid sequence at least 85% identical to any one of SEQ ID NOs: 4-6. In some embodiments, the engineered protein comprises an amino acid sequence at least 90% identical to any one of SEQ ID NOs: 1-6. In some embodiments, the engineered protein comprises an amino acid sequence at least 95% identical to any one of SEQ ID NOs: 1-6. In some embodiments, the engineered protein comprises an amino acid sequence at least 95% identical to any one of SEQ ID NOs: 4-6. In some embodiments, the engineered protein comprises an amino acid sequence of any one of SEQ ID NOs: 1-6, or a sequence thereof having 1, 2, 3, or 4 amino acid substitutions, additions, or deletions. In some embodiments, the engineered protein comprises an amino acid sequence of SEQ ID NO: 4, 5, or 6, or a sequence thereof having 1, 2, 3, or 4 amino acid substitutions, additions, or deletions. In some embodiments, the engineered protein comprises an amino acid sequence of SEQ ID NO: 4, 5, or 6, or a sequence thereof having 1 or 2 amino acid substitutions, additions, or deletions. In some embodiments, the engineered protein comprises an amino acid sequence 100% identical to any one of SEQ ID NOs: 1-6. In some embodiments, the engineered protein comprises an amino acid sequence 100% identical to any one of SEQ ID NOs: 4-6. In some embodiments, the engineered protein comprises an amino acid sequence of SEQ ID NO: 4. In some embodiments, the engineered protein comprises an amino acid sequence of SEQ ID NO: 5. In some embodiments, the engineered protein comprises an amino acid sequence of SEQ ID NO: 6. In some embodiments, the engineered protein comprises one or more amino acid residues that interact with one or more amino acid residues of an Eph receptor. In some embodiments, the Eph receptor is an Eph A receptor. In some embodiments, the Eph receptor is an Eph B receptor. In some embodiments, the engineered protein comprises one or more amino acid residues that interact with one or more amino acid residues of more than one Eph receptor. In some embodiments, the more than one Eph receptor is selected from EphA2, EphA3, EphA4, EphA5, EphA6, EphA7, EphA8, EphA10, EphB1, EphB2, EphB3, EphB4, and EphB6. In some embodiments, the more than one Eph receptor is selected from EphA2, EphB2, and EphB3. In some embodiments, the engineered protein comprises a protein sequence wherein an amino acid ASN at an aligned position 1 of SEQ ID NO: 5 interacts with a VAL residue in an EphB2 receptor at an aligned position of VAL94 from SEQ ID NO: 101. In some embodiments, the engineered protein comprises a protein sequence wherein an amino acid ASN at an aligned position 1 of SEQ ID NO: 5 interacts with a VAL residue in an EphB2 receptor at an aligned position of VAL156 from SEQ ID NO: 101. In some embodiments, the engineered protein comprises a protein sequence wherein an amino acid TRP at an aligned position 3 of SEQ ID NO: 5 interacts with a SER residue in an EphB2 receptor at an aligned position of SER47 from SEQ ID NO: 101. In some embodiments, the engineered protein comprises a protein sequence wherein an amino acid GLU at an aligned position 6 of SEQ ID NO: 5 interacts with a ARG residue in an EphB2 receptor at an aligned position of ARG155 from SEQ ID NO: 101. In some embodiments, the engineered protein comprises a protein sequence wherein an amino acid LYS at an aligned position 8 of SEQ ID NO: 5 interacts with a GLU residue in an EphB2 receptor at an aligned position of GLU44 from SEQ ID NO: 101. In some embodiments, the engineered protein comprises a protein sequence wherein an amino acid HIS at an aligned position 11 of SEQ ID NO: 5 interacts with a GLU residue in an EphB2 receptor at an aligned position of GLU44 from SEQ ID NO: 101. In some embodiments, the engineered protein comprises a protein sequence wherein an amino acid TYR at an aligned position 13 of SEQ ID NO: 5 interacts with a GLU residue in an EphB2 receptor at an aligned position of GLU66 from SEQ ID NO: 101. In some embodiments, the engineered protein comprises a protein sequence wherein an amino acid ILE at an aligned position 15 of SEQ ID NO: 5 interacts with a CYS residue in an EphB2 receptor at an aligned position of CYS62 from SEQ ID NO: 101. In some embodiments, the engineered protein comprises a protein sequence wherein an amino acid ILE at an aligned position 15 of SEQ ID NO: 5 interacts with a PHE residue in an EphB2 receptor at an aligned position of PHE65 from SEQ ID NO: 101. In some embodiments, the engineered protein comprises the amino acid sequence of SEQ ID NO: 4, 5, or 6. In some embodiments, the engineered protein binds an ephrin receptor.

### Pharmaceutical Compositions:

In some embodiments, the pharmaceutical composition described herein is provided for preventing, inhibiting the progress of, treating, or ameliorating one or more symptoms of Alzheimer's disease in a subject. In some embodiments, a pharmaceutical composition described herein is provided for preventing, inhibiting the progress of, treating, or ameliorating one or more symptoms of TBI in a subject. In some embodiments, a pharmaceutical composition described herein is provided for preventing, inhibiting the progress of, treating, or ameliorating one or more symptoms of an optic neuropathy in a subject. In some embodiments, the optical neuropathy comprises glaucoma. Provided herein, in certain aspects, are pharmaceutical compositions that prevent or inhibit the progress of a tauopathy in a subject.

In some embodiments, the pharmaceutical compositions described herein prevent or inhibit the progression of Alzheimer's disease in a subject. Typically, Alzheimer's disease typically progresses in three stages—early (mild), middle (moderate), and late (severe). In the early (mild) stage of Alzheimer's disease, patients may experience difficulty performing complex tasks, may lose or misplace belongings, or have difficulty organizing or planning. In the middle (moderate) stage of Alzheimer's disease, patients may experience memory loss of events or personal history, depression, having trouble controlling their bladder and bowels, wandering and getting lost, or exhibit personality changes. In the late (severe) stage of Alzheimer's disease, patients may experience difficulty in communicating or inability to walk without assistance. In advanced stages of AD, the progression of the disease can be fatal. In some embodiments, the pharmaceutical compositions described herein prevent or inhibit the progress of early-stage Alzheimer's disease in a subject. In some embodiments, the pharmaceutical compositions described herein prevent or inhibit the progress of middle-stage Alzheimer's disease in a subject. In some embodiments, the pharmaceutical compositions described herein prevent or inhibit the progress of late-stage Alzheimer's disease in a subject.

In an aspect, pharmaceutical compositions described herein comprises compositions disclosed herein formulated together with one or more pharmaceutically acceptable carriers. In some embodiments, the pharmaceutical composition comprises a composition disclosed here formulated together with one or more pharmaceutically acceptable excipients. In some embodiments, a pharmaceutical composition comprising an engineered protein described herein formulated together with a pharmaceutically acceptable carrier is used in a method of treating a neurodegenerative condition in a subject. In some embodiments, a pharmaceutical composition comprising a peptide ligand described herein formulated together with a pharmaceutically acceptable carrier is used in a method of treating a neurodegenerative condition in a subject. In some embodiments, a pharmaceutical composition comprising an ephrin ligand mimetic peptide described herein formulated together with a pharmaceutically acceptable carrier is used in a method of treating a neurodegenerative condition in a subject. In some embodiments, a pharmaceutical composition comprising an pan-Eph receptor Ephrin ligand mimetic peptide described herein formulated together with a pharmaceutically acceptable carrier is used in a method of treating a neurodegenerative condition in a subject.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers. In some embodiments, the pharmaceutical compositions described herein prevent or inhibit the progress of Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, or Hallevorden-Spatz disease in a subject.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing pharmaceutical compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the pharmaceutical compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses. The phrase “a therapeutically effective amount” also refers to an amount of an ephrin ligand mimetic peptide described herein that results in a measurable therapeutic response. A therapeutic response may be any response that a user of the method (e.g., a clinician) will recognize as an effective response to the therapy, including improvement of one or more symptoms (e.g., one or more symptoms a neurodegenerative condition) and surrogate clinical markers (e.g., neurodegeneration biomarkers). A therapeutic response will generally be an amelioration or inhibition of one or more symptoms of a disease or condition, (e.g., a form of neurodegeneration such as AD, TBI, an optic neuropathy, glaucoma, or stroke). Measurable therapeutic response also includes a finding that one or more symptoms of a disease or a disease or disorder is prevented or has a delayed onset, or is otherwise attenuated by a therapeutic agent described herein (e.g., an ephrin ligand mimetic peptide), thus, a “therapeutically effective amount” as used herein refers to an amount sufficient to reduce one or more symptom(s) or condition(s) associated with a neurodegenerative condition.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

### Methods

### Treatment of Diseases Such as Neurodegenerative Diseases, Eye Diseases or Cancer

In some embodiments, the subject has a neurodegenerative disease. The term neurodegenerative disease includes, but is not limited to, Alzheimer's disease (AD), traumatic brain injury (TBI), spinal cord injury (SCI), neuropathy, optic neuropathy, glaucoma, stroke, or other Central Nervous System (CNS) neurodegenerative diseases. In some embodiments, the neurodegenerative disease is a tauopathy. In some embodiments, compositions described herein are provided for preventing, inhibiting the progress of, treating, or ameliorating one or more symptoms of a tauopathy in a subject. In some embodiments, pharmaceutical compositions described herein are provided for preventing, inhibiting the progress of, treating, or ameliorating one or more symptoms of a tauopathy in a subject. Tauopathies are neurodegenerative diseases caused by abnormal aggregation of tau proteins in the form of neurofibrillary tangles (NFT) in the human brain. Examples of tauopathies include, but are not limited to, Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, or Hallevorden-Spatz disease.

In some embodiments, the subject has an eye disease or disorder (both of which may be used interchangeably). In some embodiments, a composition described herein, such as an peptide or protein, may be used to treat the eye disorder. In some embodiments, the eye disorder comprises glaucoma. In some embodiments, the composition or peptide, or the administration thereof, reduces intraocular pressure in an eye of the subject. In some embodiments, the intraocular pressure is reduced by at least 10%, relative to a baseline intraocular pressure measurement.

In some embodiments, the methods described herein comprising administering a composition described herein to a subject in need thereof prevents or inhibits the progress of early-stage Alzheimer's disease in a subject. In some embodiments, the methods described herein comprising administering a composition described herein to a subject in need thereof prevent or inhibit the progress of middle-stage Alzheimer's disease in a subject. In some embodiments, the methods described herein comprising administering a composition described herein to a subject in need thereof prevent or inhibit the progress of late-stage Alzheimer's disease in a subject. In some embodiments, the methods described herein comprising administering a composition described herein to a subject in need thereof improves one of more symptoms of early-stage Alzheimer's disease in a subject. In some embodiments, the methods described herein comprising administering a composition described herein to a subject in need thereof improves one of more symptoms of middle-stage Alzheimer's disease in a subject. In some embodiments, the methods described herein comprising administering a composition described herein to a subject in need thereof improves one of more symptoms of late-stage Alzheimer's disease in a subject. The degree of neurofibrillary tangle involvement AD is defined by Braak stages 1-6. Braak staging can be used to determine the if AD is at an early-stage, middle-stage, or late-stage in the subject. In Preclinical stage 1 of AD: evidence of amyloidosis is found through PET imaging or CSF analysis. In Preclinical stage 2 of AD: evidence of amyloidosis and neurodegeneration is found through PET imaging and CSF analysis. In Preclinical stage 3 of AD: evidence of amyloidosis, neurodegeneration, and subtle cognitive changes are found. The prodromal stage of AD can be referred to as mild cognitive impairments (MCI) due to AD. The prodromal stage of AD can be referred to as mild cognitive impairments (MCI) due to AD. At this stage, obvious symptoms of brain dysfunction manifest themselves and a subject at the prodromal stage of AD will meet the criteria for MCI and the primary underlying pathophysiology of judged by a clinician or medical professional will be considered to be due to AD. The dementia stage of AD occurs when there is significant impairments of a subject's social and occupational functioning, in some instances manifested by the loss of independence to perform activities of daily living due to cognitive impairments. Typically, AD dementia involves impairment of short term and semantic memory plus other cognitive domains (e.g., executive, language, and visuospatial functions). In some embodiments, the compositions described herein used in methods of treatment, prevent or inhibit the progress of preclinical stages of Alzheimer's disease in a subject. In some embodiments, the compositions described herein prevent or inhibit the progress of Preclinical stage 1 of Alzheimer's disease in a subject. In some embodiments, the compositions described herein prevent or inhibit the progress of Preclinical stage 2 of Alzheimer's disease in a subject. In some embodiments, the compositions described herein prevent or inhibit the progress of Preclinical stage 3 of Alzheimer's disease in a subject. In some embodiments, the compositions described herein prevent or inhibit the progress of the prodromal stage of Alzheimer's disease in a subject. In some embodiments, the compositions described herein prevent or inhibit the progress of the dementia stage of Alzheimer's disease in a subject.

In some embodiments, the methods described herein comprising administering a composition described herein to a subject in need thereof improves one of more symptoms of glaucoma. In some embodiments, symptoms of glaucoma comprise severe headache, severe eye pain, nausea, vomiting, blurred vision, halos or colored rings seen around lights, eye redness, or premature loss of retinal ganglion cells.

In some embodiments, the methods described herein comprising administering a composition described herein to a subject in need thereof improves one of more symptoms of AMD. In some embodiments, symptoms of AMD comprise blurry or fuzzy vision, Difficulty recognizing familiar faces, straight lines appear wavy, a dark or empty area or blind spot appearing in the center of vision, or loss of central vision.

In some embodiments, the methods described herein comprising administering a composition described herein to a subject in need thereof improves one of more symptoms of TBI. In some embodiments, symptoms of TBI comprise loss of consciousness from several minutes to hours, persistent headache or headache that worsens, repeated vomiting or nausea, convulsions or seizures, dilation of one or both pupils of the eyes, clear fluids draining from the nose or ears, inability to awaken from sleep, or excessive loss of CNS neurons.

The disclosure also contemplates a method of treating or preventing cancer in a subject comprising administering one or more pan-Eph receptor Ephrin ligand mimetic peptides in therapeutically effective amount to treat or prevent cancer in the subject. In various aspects the cancer includes, but is not limited to, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, pilocytic astrocytoma, schwannoma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, cutaneous melanoma, ocular melanoma, uveal melanoma, melanoma brain metastases, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL) including DLBCL following autologous stem cell transplantation, multiple myeloma, chronic myeloid leukemia, adult acute myeloid leukemia in remission; adult acute myeloid leukemia alkylating agent-related acute myeloid leukemia, chronic lymphocytic leukemia, Richter's syndrome; waldenstrom macroglobulinemia, adult glioblastoma; adult gliosarcoma, recurrent glioblastoma, glioblastoma multiforme (GBM), recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; cervical cancer, anal canal squamous cell carcinoma; metastatic anal canal carcinoma; recurrent anal canal carcinoma, recurrent head and neck cancer; head and neck squamous cell carcinoma (HNSCC), adenoid cystic carcinoma, sebaceous cell carcinoma, basal cell carcinoma, ovarian carcinoma, colon cancer, gastric cancer, advanced GI cancer, gastric adenocarcinoma; gastroesophageal junction adenocarcinoma, thyroid cancer, liver cancer, endometrial cancer, bone neoplasms, soft tissue sarcoma; bone sarcoma, retinoblastoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In various aspects, the cancer is melanoma, cutaneous melanoma, ocular melanoma, cervical cancer, follicular B cell non-Hodgkin's lymphoma, kidney cancer, prostate cancer, and multiple myeloma, breast cancer, lung cancer, colon cancer, ovarian cancer, bladder cancer, pancreatic cancer, endometrial cancer, liver cancer, thyroid cancer, leukemia.

In various aspects of the disclosure, an Ephrin ligand mimetic peptide is administered in an amount and for a time sufficient to reduce tumor volume, reduce tumor burden, and/or reduce metastasis in the mammalian subject. Tumor volume can be measured using methods such as, for example, computed tomographic (CT) scan or positron emission tomography (PET) imaging. Tumor burden can be determined by, e.g., measuring tumor markers in biological samples. In various embodiments, the method reduces tumor volume or tumor burden by at least 1%, 3%, 5%, 10%, 20%, 30%, 40%, 50% or more. In various embodiments, the method reduces tumor volume or tumor burden by at least 10%, 15%, 20%, 25%, 30%, 35%, 40%, 45%, 50%, 55%, 60%, 65%, 70%, 75%, 80%, 85%, 90%, 95% or 100%. Ranges containing any of the aforementioned integers as lower and upper ends are contemplated (e.g., 10% to 50% or 3% to 50%). It will be appreciated that complete eradication of the tumor or tumor burden is not required to achieve a beneficial response; any level of reduction of tumor burden, tumor volume, or metastasis is contemplated.

### Subject

A subject can be any mammal, such as a human. Contemplated subjects also include, but are not limited to, animals of agricultural importance, such as bovine, equine, and porcine animals; animals serving as domestic pets, including canines and felines; animals typically used in research, including rodents (e.g., mice or rats) and primates; large endangered species; and zoo animals such as primates, felines, giraffes, elephants, rhinos

### Dosing and Administration

“Therapeutically effective amount” refers, e.g., to an amount of one of more Ephrin ligand mimetic peptides which is effective, upon single or multiple dose administration, to treat or prevent neurodegenerative disease or cancer in the subject. Where the subject is suffering from a disorder, a therapeutically effective amount optionally prolongs the survivability of the subject, reduces one or more signs or symptoms of the disorder, prevents or delays onset or progression of the disorder, and the like, beyond that expected in the absence of such treatment. The dose of the Ephrin ligand mimetic peptide need not remain consistent over the course of a treatment period but can be varied until a desired effect is achieved.

In various aspects, an Ephrin ligand mimetic peptide is administered in an amount sufficient to increase regeneration of axonal projections by >500 μm distal to a crush site on a nerve. In general, a dose of Ephrin ligand mimetic peptide employed for mammalian subject treatment is in the range of 0.01 mg/kg to 5 mg/kg per administration. In related embodiments, a dose of Ephrin ligand mimetic peptide is between 0.05-0.1 mg/kg, 0.1-1 mg/kg, 1-2 mg/kg, 2-3 mg/kg, 3-4 mg/kg, or 4-5 mg/kg. In some embodiments, a therapeutically effective dose of an Ephrin ligand mimetic peptide employed for mammalian subject treatment is at least about 2 μg/kg, 5 μg/kg, 10 μg/kg, 30 μg/kg, 50 μg/kg, 75 μg/kg, 100 μg/kg, 200 μg/kg, 300 μg/kg, 400 μg/kg, 500 μg/kg, 600 μg/kg, 750 μg/kg, 1 mg/kg, 2 mg/kg, 3 mg/kg, 4 mg/kg, 5 mg/kg, or 6 mg/kg

In various aspects, an Ephrin ligand mimetic peptide is administered in combination with an adjuvant. In general, a dose of adjuvant employed for mammalian subject treatment is in the range of about 10 μg to about 5000 μg per administration. In related embodiments, the dose of adjuvant peptide is between 10-100 μg, 100-500 μg, 500-1000 μg or 1000-5000 μg per administration.

In some embodiments described herein are methods for treating a neurodegenerative condition wherein a pharmaceutical composition comprising a peptide ligand described herein is administered according to a treatment regimen. In some embodiments, the treatment regimen comprises a single dosing treatment regimen. In some embodiments, the treatment regimen comprises a multiple dosing treatment regimen. In some embodiments, the multiple treatment regimens comprise 2 or more treatments, 3 or more treatments, 4 or more treatments, 5 or more treatments, 6 or more treatments, 7 or more treatments, 8 or more treatments, 9 or more treatments, 10 or more treatments, 12 or more treatments, 15 or more treatments, 20 or more treatments, or 25 or more treatments. In some embodiments, a multiple treatment regimen will comprise between 1-2 treatments, between 1-3 treatments, between 1-4 treatments, between 1-5 treatments, between 1-6 treatments, between 1-7 treatments, between 1-8 treatments, between 1-9 treatments, between 1-10 treatments, between 1-12 treatments, between 1-15 treatments, between 1-20 treatments, between 2-3 treatments, between 2-4 treatments, between 2-5 treatments, between 2-6 treatments, between 2-7 treatments, between 2-8 treatments, between 2-9 treatments, between 2-10 treatments, between 2-12 treatments, between 2-15 treatments, between 2-25 treatments, between 3-4 treatments, between 3-5 treatments, between 3-6 treatments, between 3-7 treatments, between 3-8 treatments, between 3-9 treatments, between 3-10 treatments, between 3-14 treatments, between 3-18 treatments, between 3-30 treatments, between 4-5 treatments, between 4-6 treatments, between 4-7 treatments, between 4-8 treatments, between 4-9 treatments, between 4-10 treatments, between 4-15 treatments, between 5-6 treatments, between 5-7 treatments, between 5-8 treatments, between 5-10 treatments, between 5-14 treatments, between 5-23 treatments, between 5-34 treatments, between 6-7 treatments, between 6-8 treatments, between 6-9 treatments, between 6-10 treatments, between 6-12 treatments, between 6-17 treatments, between 6-22 treatments, between 6-30 treatments, between 7-8 treatments, between 7-9 treatments, between 7-12 treatments, between 7-18 treatments, between 9-18 treatments, between 10-20 treatments, between 12-25 treatments, between 14-35 treatments, between 15-50 treatments, or between 20-100 treatments. In some embodiments, a single dosing treatment regimen will be following a diagnosis of a neurodegenerative condition in the subject. In some embodiments, a multiple dosing treatment regimen will be following a diagnosis of a neurodegenerative condition in the subject. In some embodiments, the treatment regimen comprises administering a pharmaceutical composition described herein to the subject at a determined dosage with a frequency of one administration about 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 52 hr, 56 hr, 60 hr, 64 hr, 68 hr, 72 hr, 3.5 days, 4 days, 5 days, 6 days, 7 days, 8 days, 9 days, 10 days, 11 days, 12 days, 13 days, 14 days, 15 days, 18 days, 20 days, 21 days, 24 days, 28 days, or 30 days. In some embodiments, the post-treatment regimen comprises administering a pharmaceutical composition described herein to the subject at a determined dosage with a frequency of one administration about every other day.

In some embodiments, the post-treatment regimen comprises administering a pharmaceutical composition described herein to the subject at a determined dosage with a frequency of one administration about every other day until one or more symptoms of a neurodegenerative condition in the subject have shown improvement.

The Ephrin ligand mimetic peptide may be administered to a subject using any suitable route of administration. Suitable routes of administration include, but are not limited to, subcutaneous, intramuscular, intravenous, intraperitoneal, intratumoral, intrathecal, percutaneous, endovascular, intradermal, intra-lymph node administration.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order.

Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

The terms “treat,” “treated,” “treating,” “treatment,” and the like are meant to refer to reducing or ameliorating a disorder and/or symptoms associated therewith (e.g., a neoplasia or tumor). “Treating” may refer to administration of the combination therapy to a subject after the onset, or suspected onset, of a neurodegenerative disease or a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

As used herein, and unless otherwise specified, the terms “prevent,” “preventing” and “prevention” refer to the prevention of the onset, recurrence or spread of a disease or disorder, or of one or more symptoms thereof. In some embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In some embodiments, subjects with familial history of a disease are potential candidates for preventive regimens. In some embodiments, subjects who have a history of recurring symptoms are also potential candidates for prevention. In this regard, the term “prevention” may be interchangeably used with the term “prophylactic treatment.”

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. It is further contemplated that other adjunct therapies may be administered, where appropriate. For example, the subject may also be administered additional cytotoxic agents, biologic agents, targeted small molecule inhibitors, photodynamic therapy and/or radiation therapy, or have undergone a surgical procedure (e.g., tumor resection)

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection. Sequences can be aligned for maximum correspondence over a comparison window or designated region as measured using a BLAST or BLAST 2.0 sequence comparison algorithm with default alignment parameters, or by manual alignment and visual inspection, e.g., NCBI web site).

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 or Megalign. The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### General Methods

### Ephrin Ligand Sequence Homology and In-Silico Peptide Docking Models

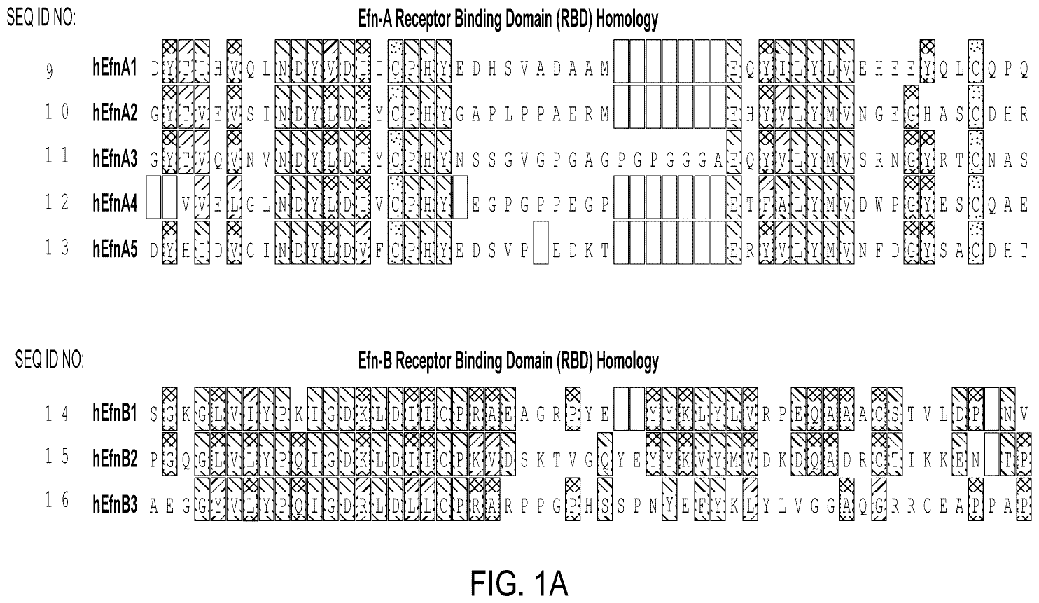

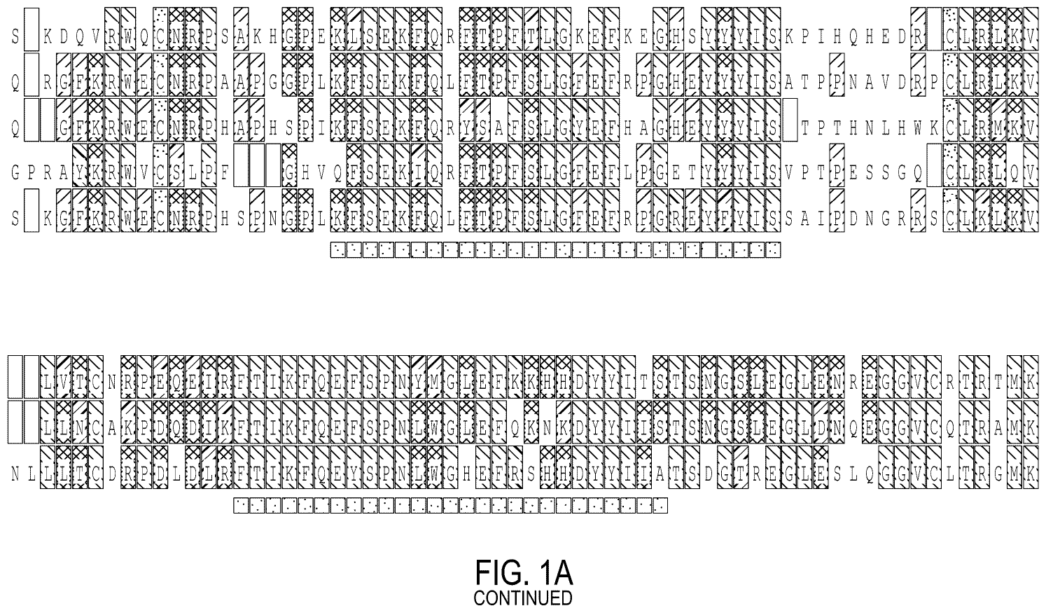

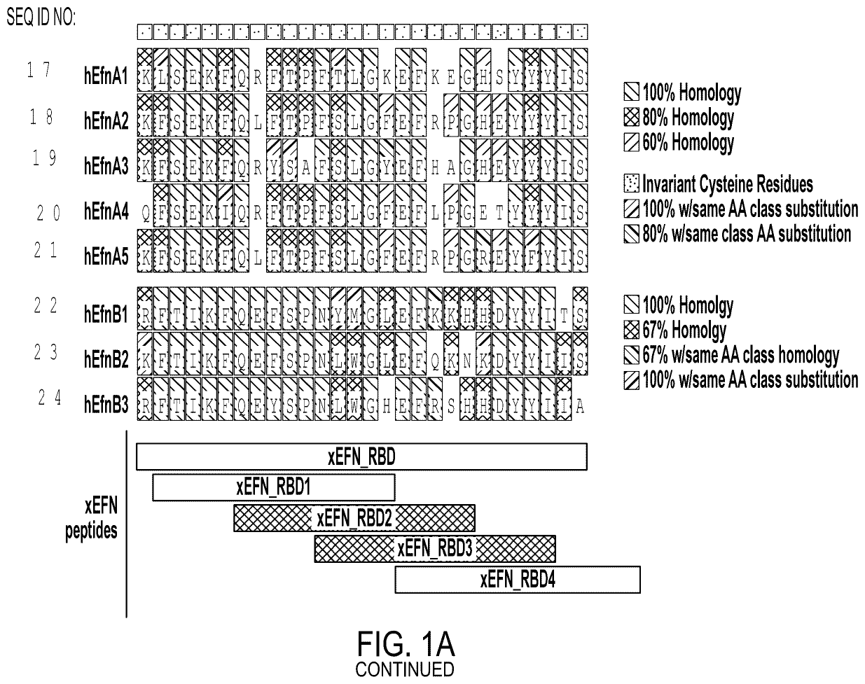

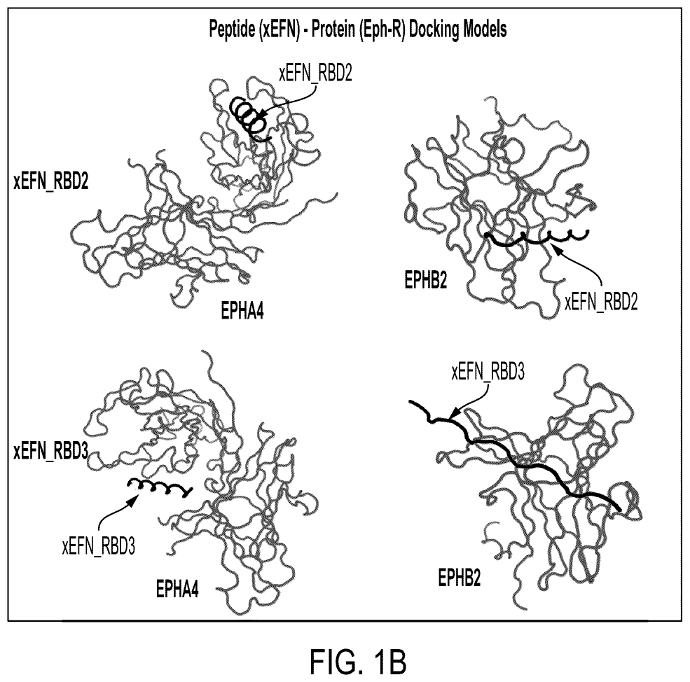

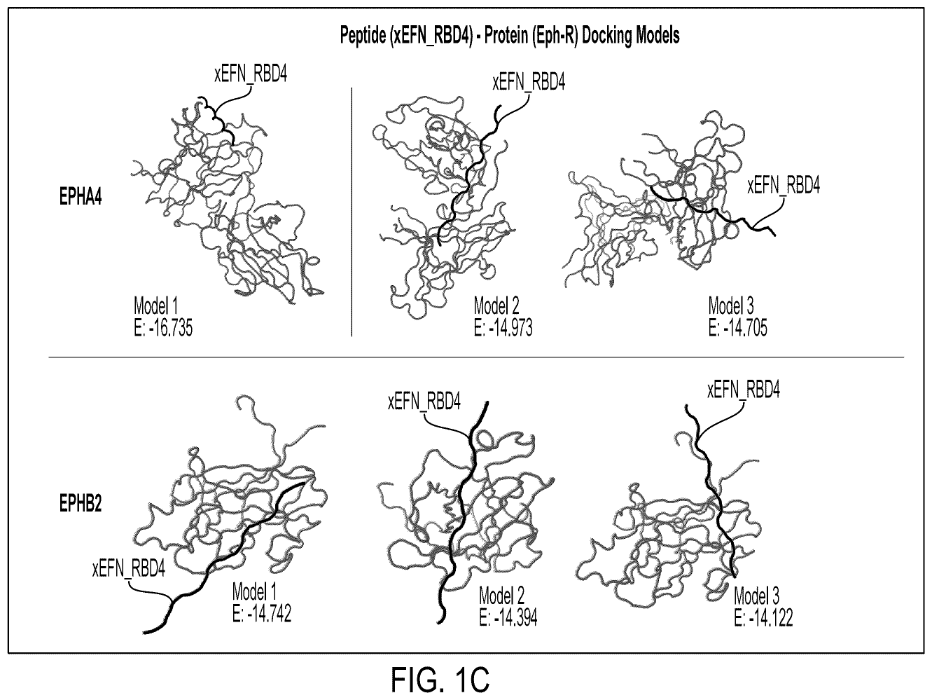

Amino acid sequences for human and mouse efn ligands were obtained from the National Center for Biotechnology Information (NCBI) (FIG. 1A, FIGS. 12A-12C), and were initially aligned to each other using the NCBI's Basic Local Alignment Search Tool (BLAST) and then manually curated to generate final alignment maps for homology search. Receptor binding domain (RBD) motifs were identified as the region containing the conserved 4 invariant cysteine residues in efnA ligands, and the Cupredoxin motif of the efnB ligands. Regions within the RBD consisting of 3 or more consecutive amino acids of complete homology among efn class A and B ligands were flagged for subsequent docking and modeling screens.

In-silico docking, affinity calculation, and residue substitution analysis of identified homologous regions in the efn RDB (xEFN) were performed using the pep-ATTRACT server tool (109, 110). Sequences were analyzed by simple alanine (Ala) substitution for every position, as well as substitution for amino acids with opposing biochemical characteristics to those naturally found in the sequence (positively charged residues for negatively charged ones, hydrophobic to hydrophilic, etc.). The modified sequences were then modelled for their ATTRACT force field energy to EphA4 [PDB; 2WO1, (90)] and EphB2 [PDB: 3ETP, (91)] and the top 10 calculated model energies were averaged to quantitate the effect of the specific amino acid substitution.

### Neuronal Cell Isolation and Culture

Fetal brain-derived neural stem cells (hNSC): In order to evaluate the effect of xEFN peptides on human neuronal cells, StemPro hNSC were purchased from ThermoFisher Scientific (Cat. no. A15654) and cultured according to the manufacturer's recommended protocol. Briefly, cells were grown in non-adherent neurosphere suspension cultures in StemPro NSC serum-free media (NSC-SFM) (Cat. no. A10509-01) supplemented with 2 mM GlutaMAX-I supplement, 6 U/mL heparin, and 200 μM ascorbic acid (Sigma-Aldrich, Cat. no. A8960). Culture media was changed every 2 days and cells were passaged every 7 days as undifferentiated hNSC. To induce differentiation into neuronal cells, hNSC neurospheres were dissociated into single-cell suspensions using StemPro Accutase for 5 minutes at 37° C. followed by mechanical pipetting. hNSC were then plated onto Geltrex-coated culture vessels at a concentration of 2×10⁴ cells/cm² in neuronal differentiation media consisting of NSC media without bFGF or EGF and supplemented with B-27 supplement (ThermoFisher Scientific) for 3 days prior to performing further in vitro assays.

Mouse retinal ganglion cells (RGC): Mouse R GCs were isolated as follows. Briefly, retinas from postnatal day 8-10 (p8-10) mouse pups were dissected and digested using a papain solution (16.5 U/mL) for 30 minutes at 37° C. Following dissociation, macrophage and endothelial cells were removed from the cell suspension by panning with the anti-macrophage antiserum (Accurate Chemical, Westbury, NY). Finally, RGCs were immunopanned in plates containing the antibody against Thy1.2 and were then released by incubation with trypsin solution. RGCs were grown on poly-L-lysine coated plates in serum-free Neurobasal media (Invitrogen, Carlsbad, CA), supplemented with B-27 supplement, CNTF (10 ng/ml), and BDNF (10 ng/ml).

### Neurite Sprouting and Extension Assay in Mouse Retinal Ganglion Cells

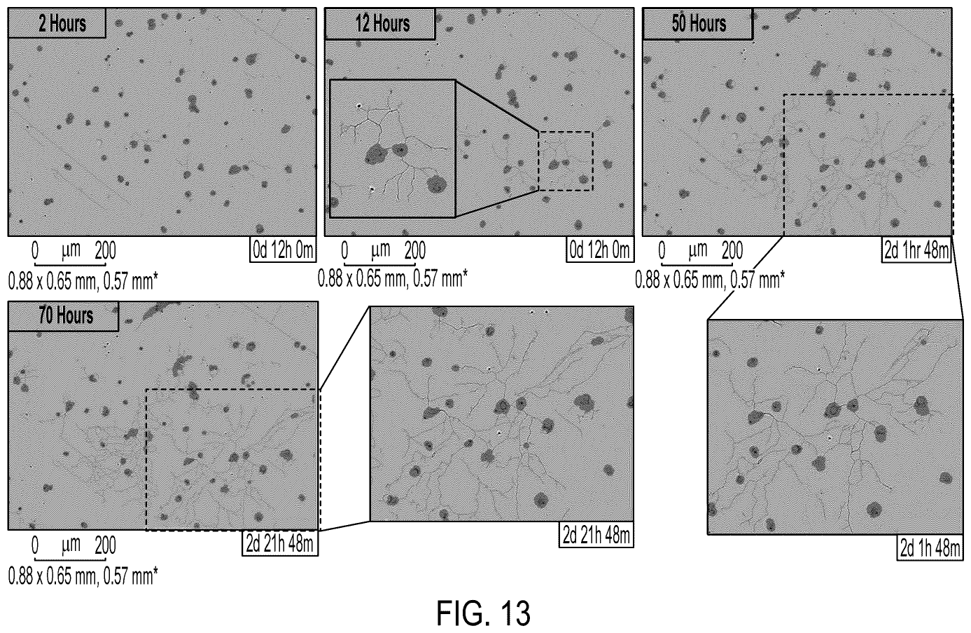

mRGCs were used to analyze dendrite sprouting, axonal polarization and extension, and ephrin receptor phosphorylation following treatment with xEFN peptides. RGCs were isolated as described above and plated onto 24 well plates at a concentration of 4×10⁴ cells/well for short-term (48 h) experiments, or into 96-well plates at a concentration of 1,000 cells/well for long-term (6 days) experiments in an Incucyte Zoom (Essen Biosciences) instrument for live cell imaging. RGC media was supplemented with vehicle (PBS), 250, 500, or 1,000 nM xEFN_RBD2, or xEFN_RBD3 peptides. For the short-tem experiment, cells were fixed in 4% paraformaldehyde for 5 minutes, permeabilized using 0.05% Triton X-100, blocked with 5% fetal bovine serum, and incubated with primary antibody against Tubulin Beta 3 Class III (TUBB3) overnight at 4° C. Cells were then washed 3×5 min with PBS and incubated with AlexaFluor488 secondary antibody for 2 h at room temperature. Cells were mounted using SlowFade Gold Antifade mounting media with DAPI (ThermoFisher) and imaged using an EVOS FL automated fluorescent microscope (ThermoFisher). Dendrite measurements and analysis was performed by a blinded investigator using ImageJ software and the Sholl analysis plugin. For the long-term experiments, groups containing two published peptides designed to specifically target the EphB2 and EphB4 receptors (SNEW and TYFD, respectively) were added in the same concentration range for comparison. Neurite sprouting and extension were quantified using the automated Incucyte Zoom NeuroTrack Processing algorithm for real-time, label-free neurite segmentation and quantitation (FIG. 13).

### Eph-Receptor Antagonism

To evaluate the effect of xEFN peptides on Eph receptor activity, enzyme-linked immunosorbent assays (ELISA), western blot analysis, immunohistochemistry staining, were used. For ELISA, 1 μg/well of a rabbit monoclonal antibody against EphA3+A4+A5 (phospho Y779+Y779+Y833) (abcam) was immobilized into Immulon 4hbx microtiter plates (ThermoFisher), and then blocked using 5% bovine serum albumin (BSA) for 2 hours under constant agitation. Protein lysates from mRGCs treated with PBS, pre-clustered recombinant mouse EfnA2 (R&D Systems), or 500 nM xEFN_RBD2 or xEFN_RBD3 for 4 days was normalized to 1 mg/ml as determined by BCA assay (Pierce), and 10 μg of total protein was incubated in the antibody pre-adsorbed wells for 4 hours in a binding buffer with ionic strength of 300 mM. ELISA wells were then washed 2× in binding buffer to remove unbound proteins. Wells were then incubated in an HRP-conjugated secondary antibody recognizing phosphorylated tyrosine residues (pTyr, Cell Signaling Technology) for 2 hours at room temperature. Finally, 50 μL of chemiluminescent substrate (Pierce) was added to each well and incubated for 10 minutes. Chemiluminescence readouts were carried out using a Spectromax i3 multimode plate reader (Molecular Devices).

For western blot analysis, mRGCs were treated with PBS (controls), 500 nMxEFN_RBD2, or 500 nM xEFN_RBD3 for 4 days. On day 4, cells were treated with pre-clustered recombinant mouse EfnA5-His ligand [(+) Control, using anti-His_Tag antibody for 30 minutes] for 2 hours. Cells were then lysed in RIPA buffer containing protease and phosphatase inhibitor cocktails and maintained on ice. Protein was quantified using a BCA assay, and 10 μg of total protein was run on a TGX stain-free Any kD gel (BioRad) and transferred using the trans-blot turbo system (BioRad). Following transfer and blocking in 5% nonfat milk for 1 h, blots were probed with antibodies against RhoA, phosphorylated Rac1, ARHGEF15, Ephexin-1, or beta-actin as an endogenous loading control overnight at 4° C. Blots were then washed 3×5 min in PBS-T and incubated in the appropriate HRP-conjugated secondary antibody for 2 h at room temperature. Blots were imaged using chemiluminescent ECL substrate (Pierce) on a BioRad ChemiDoc MP imaging system and software.

### RGC Growth Cone Collapse Analysis

One of the first signs of neuronal dysfunction is axonal growth cone collapse, and it is one of the mechanisms through which Eph-receptors exert their repulsive stimuli for axonal guidance. To assess the effect of xEFN peptides on growth cone dynamics, mRGCs were plated onto poly-L-lysine coated wells and allowed the cells to extend their processes for 48 h in media supplemented with either vehicle control (PBS), or 500 nM xEFN_RBD3 peptide. At the 48 h time point, cells were stimulated by the addition of 2 μg of pre-clustered recombinant mouse EfnA5-His (R&D Systems) for 1 hour. Following this stimulation, cells were fixed in 4% paraformaldehyde for 5 minutes, permeabilized with 0.05% Triton X-100, and blocked with 5% FBS for 1 h. Cells were then incubated in primary antibody against TUBB3 overnight at 4° C., washed, and incubated in AlexaFluor 488-conjugated secondary for 2 h at room temperature. After washing, cell cytoskeleton was counterstained with AlexaFluor 594-conjugated phalloidin for 30 min at 37° C. (ThermoFisher) and mounted with SlowFade Gold Anti-fade media with DAPI. Cells were then imaged using an EVOS FL Auto fluorescent microscope. Images were analyzed in ImageJ by a blinded investigator and a growth cone collapse was calculated manually.

### Human Fetal Brain Neural Stem Cell Growth on Ephrin-Ligand Repulsive Substrates

Glial-bound efn ligands have been associated with mediating the major repulsive milieu for neuronal process in the CNS. In SCI, for example, transgenic knockout of efnB2 ligand specifically in the glial scar was found to allow for axonal processes to extend beyond the site of injury. Moreover, assays on the repulsive nature of efn ligand-presenting surfaces have been a mainstay in axonal guidance studies for many decade. The use of xEFN peptides to antagonize this repulsive signaling from efn ligand substrates was evaluated on human fetal brain-derived neural stem cells (hNSC).

Recombinant Efn ligands (A2, A4, and B3) containing a His-Tag were purchased from R&D Systems, and were pre-clustered using an anti-His antibody (Genetex) for 30 min. The pre-clustered Efn ligands, or anti-his antibody alone, (control) were then mixed with laminin from human placenta (Sigma-Aldrich) in suspension and used to coat the growth surface of a 24-well plate. After coating, hNSC 2,500 cells/cm² were seeded into the wells and allowed to grow on the surface for a period of 5 days in StemPro neuronal differentiation medium. At the end of the 5-day culture, cells were fixed in 4% PFA, and stained with AlexaFluor 594-conjugated phalloidin. The entire wells were then scanned using an EVOS FL Auto fluorescent microscope and the images were analyzed in the ImageJ software to calculate cell coverage area.

In Vivo Use of xEFN Peptides in a Mouse Model of Optic Nerve Crush (ONC)

Antagonizing forward Eph signaling has been proven beneficial in a number of CNS disorders (Neurogastroenterol Motil. 2020:e13782; Acta Neuropathol Commun. 2018; 6(1):111; Proc Natl Acad Sci USA. 2014 Jul. 8; 111(27):9959-64; Eur J Neurosci. 2014 Oct; 40(7):3021-31; Neuroscience. 2013 Jun. 25; 241:89-99). The use of xEFN peptides as multi-Eph receptor antagonists was evaluated in treating retinal degeneration induced by optic nerve crush (ONC). The ONC model is the most commonly used model for the study of glaucoma and other optic neuropathies. All animal experiments were performed in compliance with the National Institutes of Health (Bethesda, MD, USA) Guide for the Care and Use of Laboratory Animals and the ARVO Statement for the Use of Animals in Ophthalmic and Vision Research. Animal protocols were reviewed and approved by the Institutional Animal Care and Use Committee of the University of Miami. To induce ONC, mice (12 wk old) were anesthetized, and the optic nerve was surgically accessed through the superior conjunctiva with careful blunt dissection of the orbital contents. With the use of jeweler's forceps (Dumont No. 5; tip dimension, 0.1×0.06 mm), the optic nerve is then crushed for 10 seconds at a location 2 mm distal from its insertion point into the globe. Immediately after ONC, mice were administered a 1.5 μL intravitreal injection of either vehicle (PBS control, n=3), xEFN_RBD2 (4 mg/ml, n=3), or xEFN RBD3 (4 mg/ml, n=3) and allowed to recover. Following 1 week after ONC, animals were administered a second intravitreal injection of the same compounds and then allowed to progress through 4-weeks post-ONC. 2-days prior to euthanasia (day 14 post-ONC), all animals received an intravitreal injection of AlexaFluor 647-conjugated cholera toxin subunit B (CTB, ThermoFisher) to label neuronal projections. At 4 weeks post-injury, animals were euthanized and enucleated for extraction of the retinas and optic nerves. Retinas were stained by immunohistochemistry for TUBB3 and RGCs were manually counted in each animal. Optic nerves were placed into tissue clearing solution for 1 week and then imaged for the CTB-647 label as whole mounts using a Leica SP8 Laser Scanning Confocal microscope (Leica Microsystems). Axonal projections were quantified at 100 μm intervals along the optic nerve track for up to 2 mm past the crush site using the fluorometric intensity measurement and analysis tool in the Leica LAS X microscopy suite software package, using the contralateral nerve (not CTB-injected) as a background reading for 647 nm fluorometric intensity in each sample.

### Statistical Analysis

Unless otherwise stated, all experiments were run as biological triplicates with at least 2 technical replicates of each used for analysis. One-way ANOVA was used for multiple comparisons. Student's t-test was conducted for single comparisons. P values: S 0.05 were considered to be statistically significant.

### Example 1: xEFN Mapping Region Constitutes a Cross-Class Ephrin Ligand Homologous Motif of the Receptor Binding Domain (RBD)

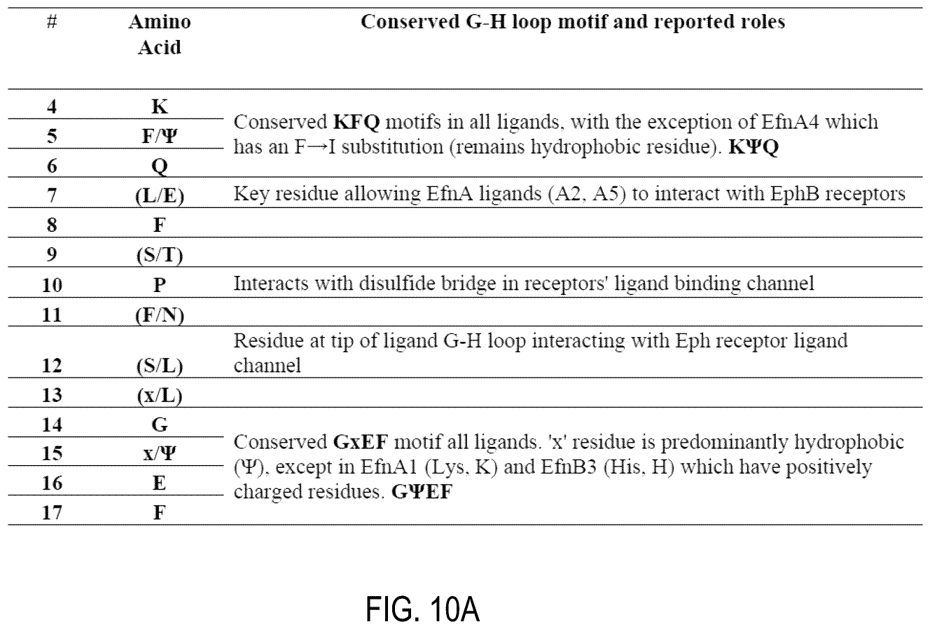

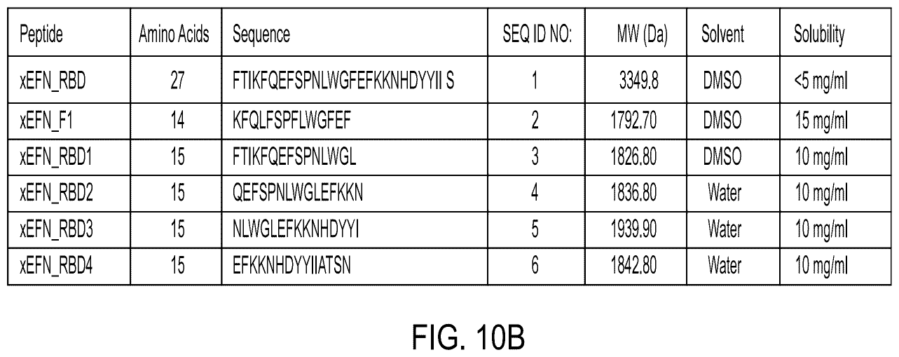

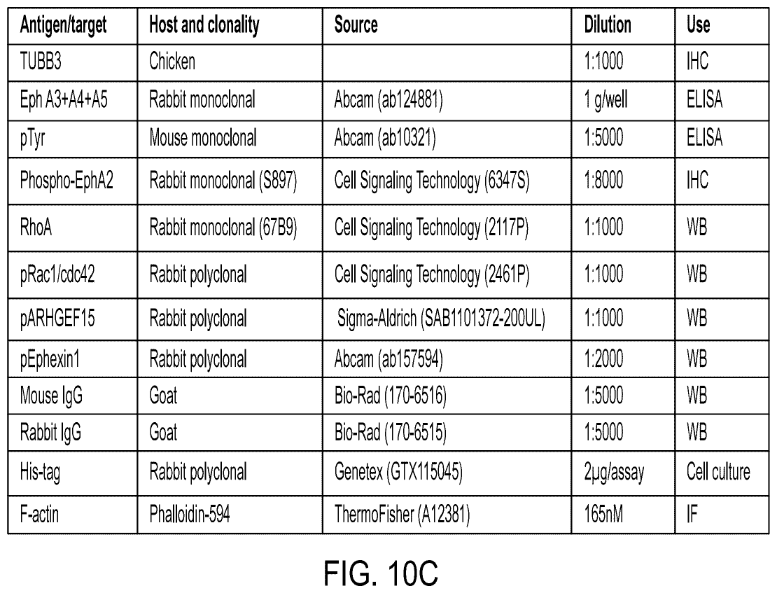

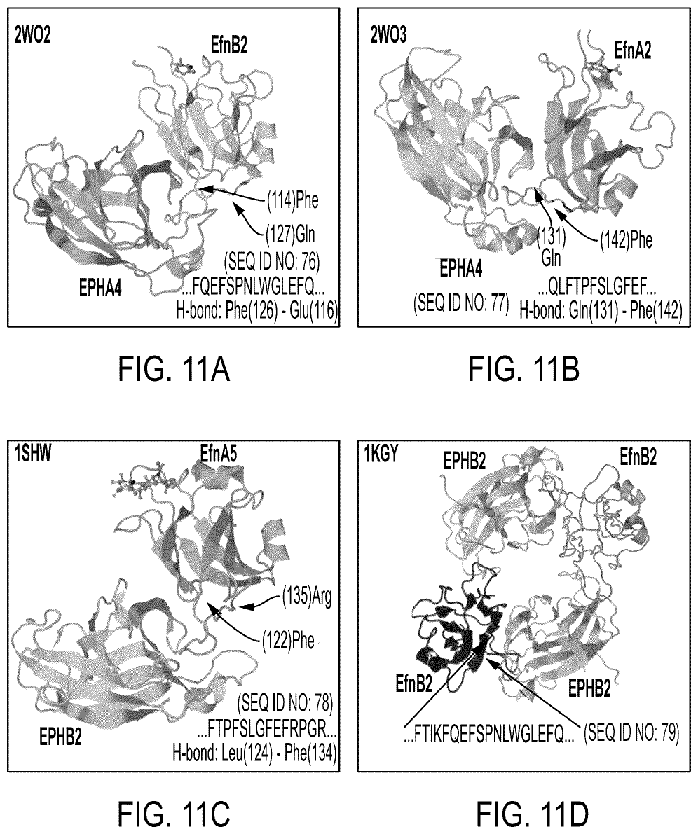

The receptor binding domain (RBD) for ephrin-A (efnA) and ephrin-B (efnB) ligands has previously been well-characterized. The efnA RBD is found within the extracellular region of the protein constrained by 4 invariant cysteine residues present in all efnA members. Similarly, the RBD for efnB family ligands resides within the cupredoxin domain of the extracellular portion of the ligand. The alignment analysis shows a high degree of class-specific homology within these RBD regions in efn ligands (FIGS. 1A, 1B). This analysis allowed for the construction of mimetic peptide libraries for both efnA (xEFNA; 27 peptides; 10 amino acids), as well as efnB (xEFNB; 6 peptides; 15 amino acids) classes that mapped to the most homologous regions within each efn ligand class. Screening of these libraries on a dendrite sprouting assay using human fetal hippocampal neural stem cells (hNSC) demonstrated that motifs within the highest homology regions conferred the strongest biological effect on neuronal processes from both xEFNA and xEFNB mimetic peptide libraries. Interestingly, upon alignment of the region with highest degree of dendrite sprouting in the hNSC assay, a region of pan-efn class homology was found (xEFN_RBD mapping region, FIG. 1C). This 27 amino acid sequence (F-x-x-K-F-Q-(L/E)-F-(S/T)-P-(F/N)-(S/L)-(x/L)-G-x-(E/A)-F-x-x-x-x-x-Y-Y-(Y/I)-I-S) is part of the G-H loop found in the RBD of efn ligands, and contains a 14 amino acid sequence of conserved motifs with key residues that have been previously reported to participate in specific interactions conferring ligand-receptor affinities (FIG. 10A). In FIG. 1C, the top three docking conformations and model estimated energies for xEFN_RBD4 with EphA4 on the top row and EphB2 on the bottom row demonstrate possible interactions between this ephrin ligand memetic peptide with different subclasses of Eph receptor.

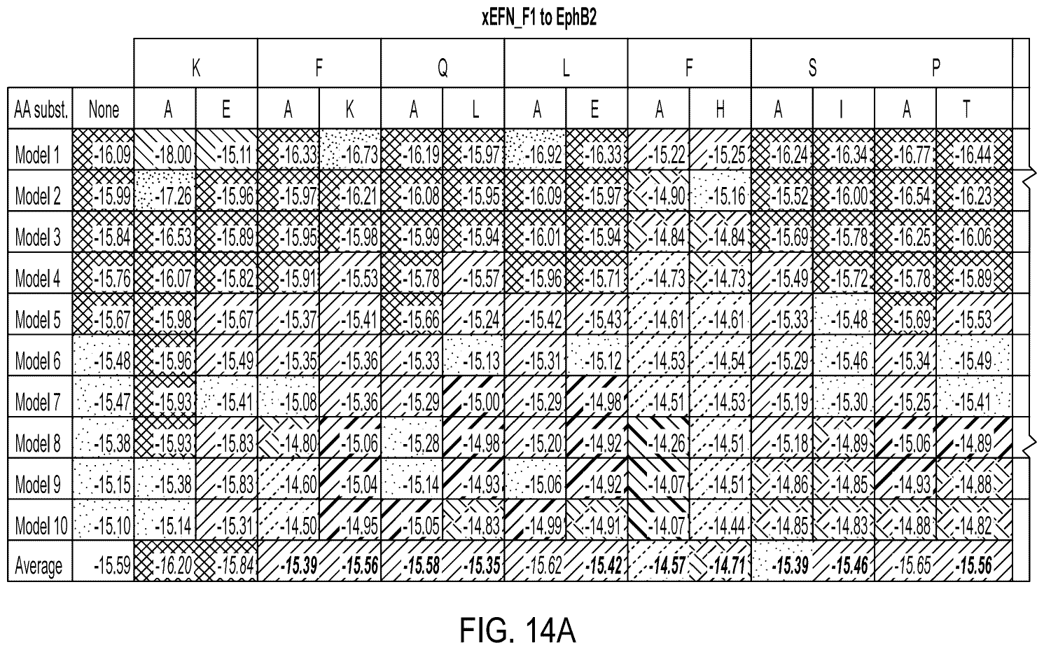

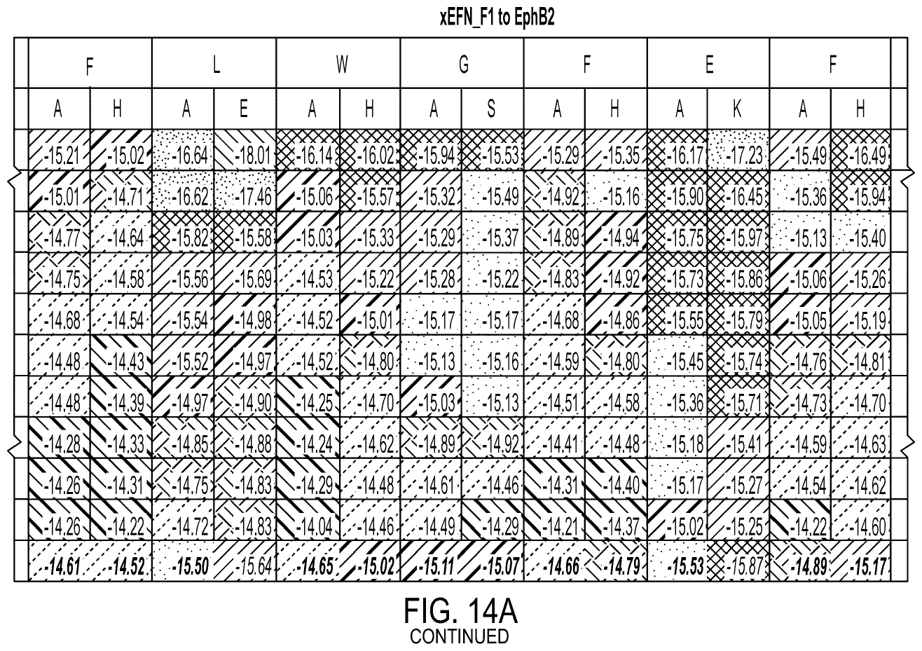

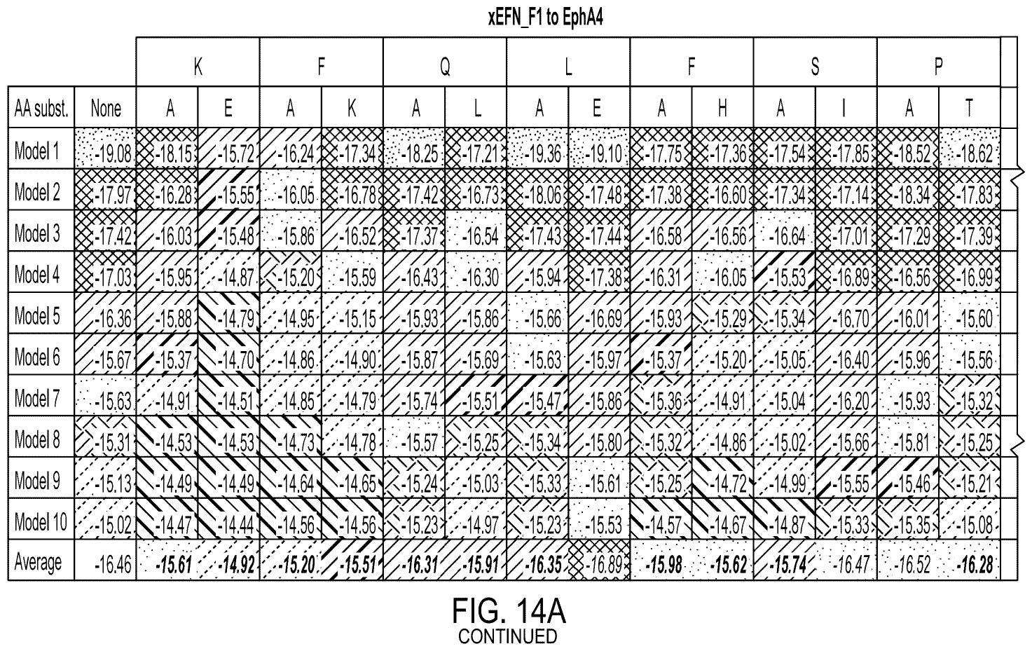

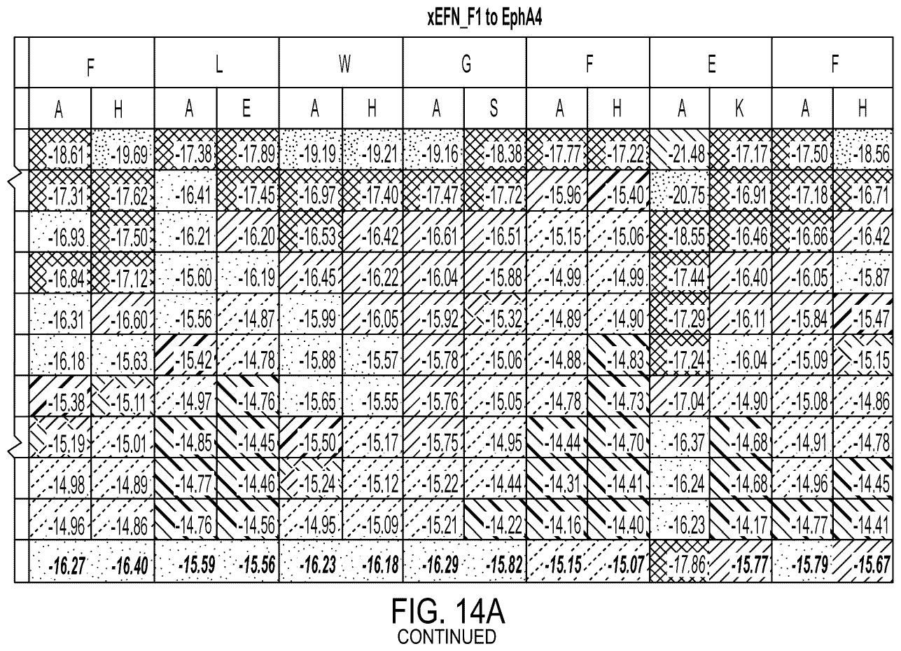

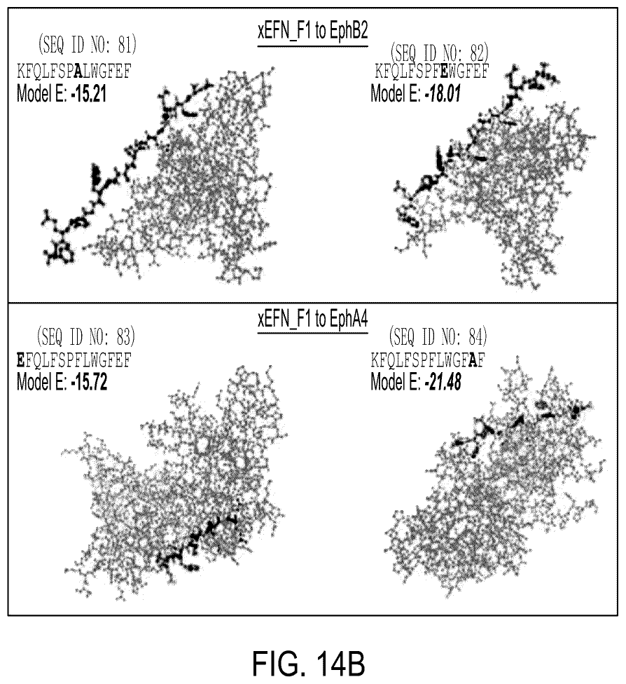

In-silico residue substitution analysis on the xEFN-F1 homologous efn RDB region showed critical amino acid residues conferring cross-class promiscuity as well as affinity of the RBD sequence to the respective receptor (FIG. 14A), visualizations of some of these substitutions were generated to show model docking (FIG. 14B). While particular substitutions yielded lower model energies for either EphB2 or EphA4 interactions with xEFN_F1, no single substitution within the xEFN_F1 sequence was shown to confer lower resting energies in the docking to both EphB2 and EphA4 (FIG. 14A). However, substitution of conserved residues in the above-mentioned motifs resulted in lower affinity energies of the xEFN_F1 sequence to both EphB2 and EphA4, further confirming the importance of these motifs in conferring cross-class promiscuity and affinity to the sequence. The xEFN_RBD sequence was used as a template for a final peptide library (FIG. 1A, FIG. 10B).

### Residue Substitution Analysis

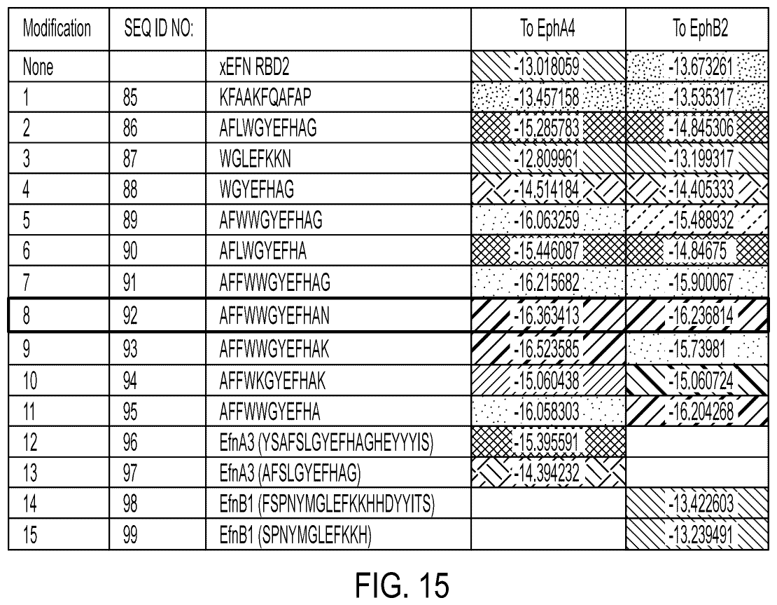

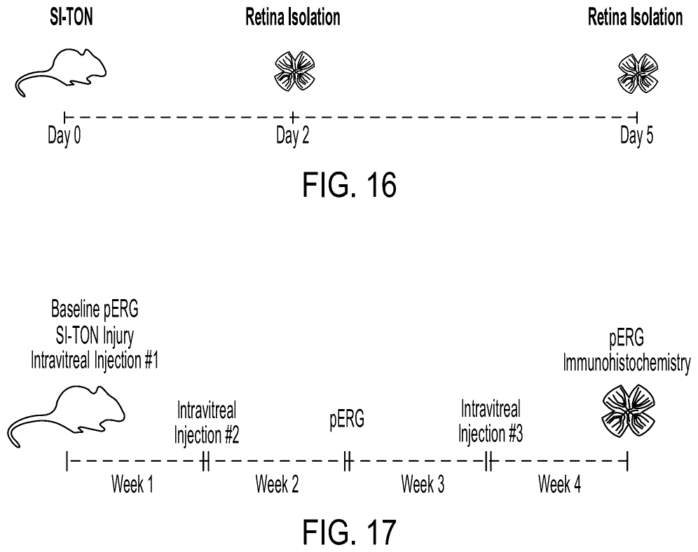

Using a tri-peptide stepwise approach based on the sequence of xEFN_RBD2, followed by in-silico docking and binding affinity averaging of the energies for the top 10 docking models generated by the pep-ATTRACT server tool identified an 8-amino acid sequence (WGYEFHAG, FIG. 15, line 4) with a calculated higher affinity to both EphA4 and EphB2 than the full length xEFN_RBD2 peptide. Furthermore, this analysis yielded peptide sequences of 10-12 amino acids in length (FIG. 15, lines 5-11) with higher binding affinities to the EphA4 and EphB2 receptors than both the xEFN_RBD2 peptide sequence or their homologous regions in the natural human ligands efnA3 and efnB1.

### Example 2: xEFN Peptides Promote Dendrite Sprouting and Arborization, and Axonal Extension in Retinal Ganglion Cells

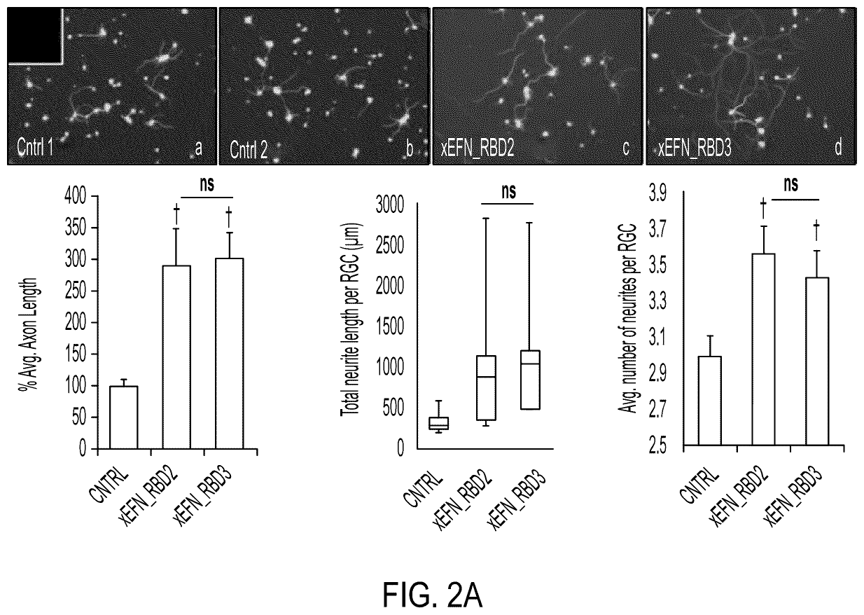

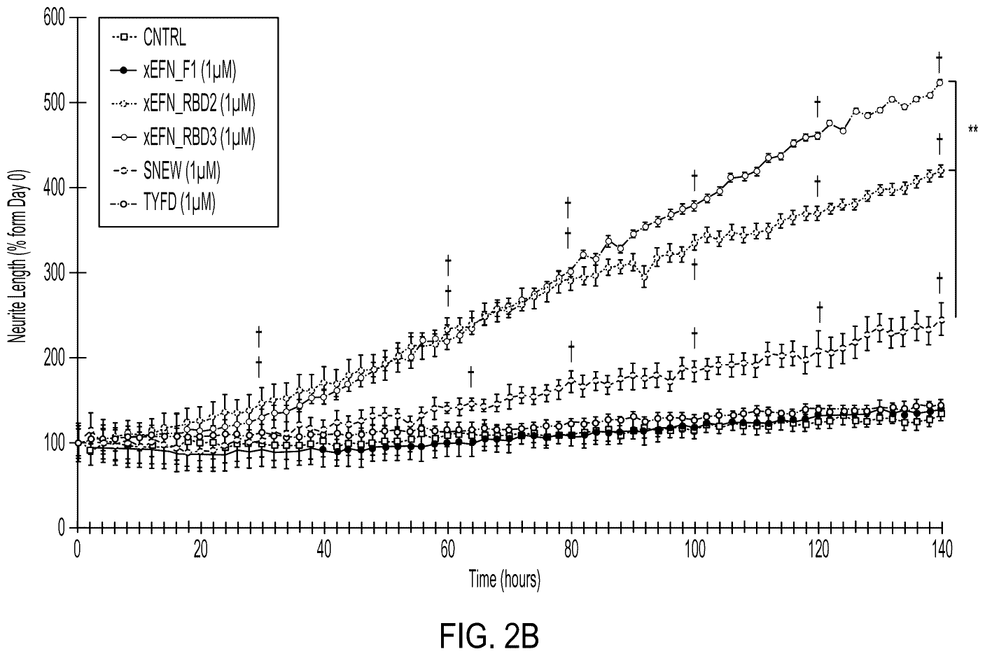

The effect of lead xEFN peptides, RBD2 and RBD3, were explored on neurite outgrowth from isolated mouse RGCs in vitro over short (48 h) and long term (6 days) growth. The data show that supplementation of RGC media with 500 nM xEFN_RBD2 or xEFN_RBD3 peptides results in a significant increase in the average axon length, total overall neurite length, and average number of sprouting neurites per RGC over a 48 h period (FIG. 2A) when compared to vehicle (PBS) supplemented RGC cultures. After 48 h of culture, RGCs supplemented with 500 nM xEFN_RBD2 or xEFN RBD3, presented with an average axonal length of 287.74+/−60.89% and 302.16+/−40.24%, respectively, when compared to vehicle (PBS) treated controls (FIG. 2Ae, p<0.01). Similarly, the median total neurite length in control RGC cultures was measured at 294.68 μm/cell (range: 209.67-589.95), while in RBD2 and RBD3 supplemented cultures total neurite length was calculated to be 888.56 (range: 289.76-2818.37) and 1034.68 (range: 486.26-2759.84) μm/cell, respectively (FIG. 2Af, p<0.05).

Finally, control RGCs had an average of 2.99+/−0.12 neurites/RGC soma, while RBD2 and RBD3 supplemented cultures had 3.58+/−0.15 and 3.43+/−0.16 neurites/RGC, respectively (FIG. 2A, p<0.05).

Over longer-term cultures, the effect of xEFN peptides on neurite extension from mRGCs was found to be more pronounced and lasting. As seen in the short-term experimentation, the supplementation of the media with 1 μM RBD2 and RBD3 xEFN peptides achieved a statistically-significant increase in neurite length over control cultures after 32 hours of culture (FIG. 2B). Over the 6-day period, supplementation of RGC media with 1 μM xEFN_RBD2 or xEFN_RBD3 also significantly increased neurite outgrowth from RGCs when compared to media supplemented with 1 μM of xEFN_F1 peptide consisting of the conserved 14 amino acid sequences described in FIG. 10A (see also FIG. 2B). Interestingly, both xEFN_RBD2 and xEFN_RBD3 peptides showed a statistically significant increase in neurite outgrowth when compared to the EphB2- or EphB4-restricted targeting peptides SNEW and TYFD at equal concentrations. By the end of the 6-day experimentation period, control RGCs achieve a 33.46+/−8.47% increase in overall neurite length over their day 0 measurement, while RGCs supplemented with 1 μM xEFN_RBD2, RBD3, or SNEW peptides achieved increases of 320.12+/−7.27, 424.68+/−4.47, and 145.31+/−18.48%, respectively (FIG. 2B, p<0.01). These results demonstrate that treatment with xEFN_RBD2 or xEFN_RBD3 significantly increases neurite sprouting in isolated RGCs. These results demonstrate that treatment with xEFN_RBD2 or xEFN_RBD3 significantly increases axonal extension in isolated RGCs. These results demonstrate that treatment with xEFN_RBD2 or xEFN_RBD3 significantly increases dendritic arbor complexity in isolated RGCs.

### Example 3: xEFN Peptides Antagonize Eph-Receptor Activation, Promote Dendritic Sprouting, and Prevent Axonal Growth Cone Collapse in RGCs

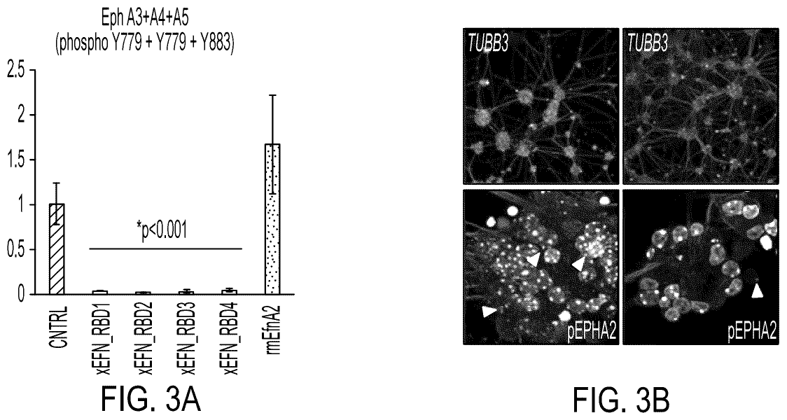

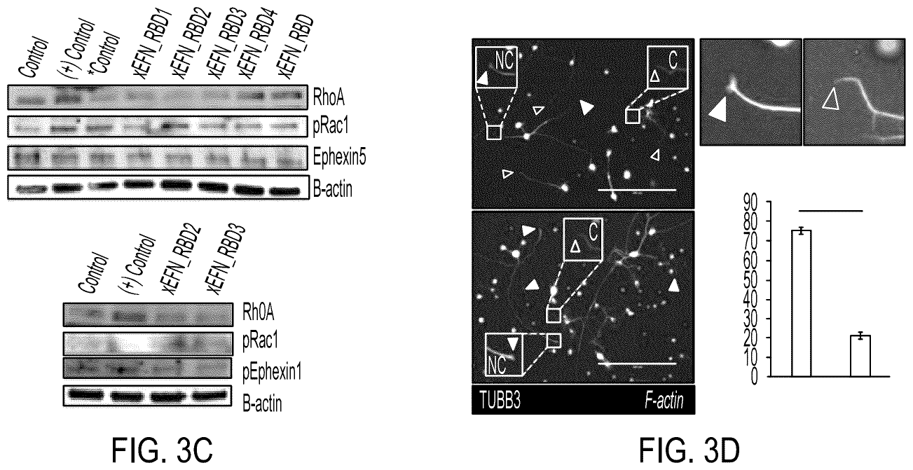

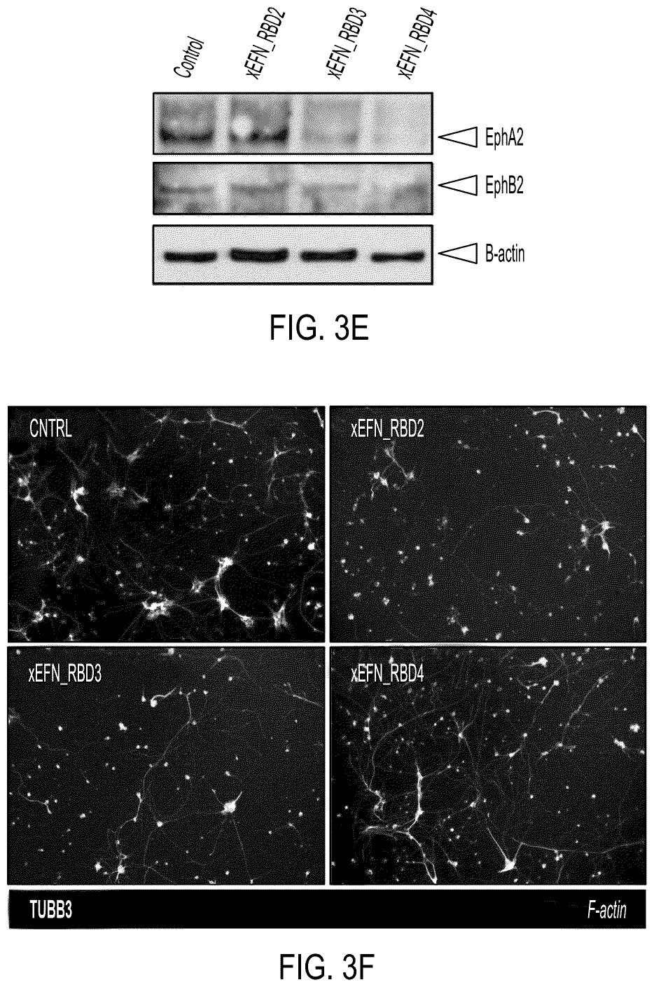

In order to assess the effect of xEFN peptide supplementation on Eph-receptor modulation, various in vitro assays were employed on isolated mouse RGCs. ELISA results show that supplementation of culture media with 500 nM xEFN_RBD1-4 for 4 days leads to a significant decrease in phosphorylation of receptors EphA1, A2, and A3 (FIG. 3A, p<0.001). This was confirmed at the cellular level via immunohistochemistry, which showed an increase dendritic density and reduced phosphorylation of the EphA2 receptor in RGCs supplemented with 500 nM xEFN_RBD3 for 4 days (FIG. 3B). Western blot analysis showed that 500 nM supplementation with xEFN_RBD1, 2, and 3 downregulates expression of the GTPase RhoA, while xEFN_RBD4 results in an upregulation of this marker. Similarly, xEFN_RBD1, and 3 inhibited the phosphorylation of Rac1 by pre-clustered ephirnA5 ligand stimulation. Finally, supplementation with xEFN_RBD2 and 3 resulted in a decrease in Eph-receptor downstream GEFs ARHGEF15 (Ephexin-5) and phosphorylation of Ephexin-1 (FIG. 3C). Analysis of growth cone dynamics demonstrates that supplementation of media with 500 nM xEFN_RBD3 for 4 days results in a significant decrease in the number of collapsed growth cones in mouse RGC cultures (FIG. 3D, p<0.001). Analysis of EphA2 and EphB2 expression by Western blot was undertaken following treatment of isolated mouse RGCs in vitro with xEFN_RBD2, xEFN_RBD3, or xEFN_RBD4. The effect of 4 days of 1 μM treatment with either xEFN_RBD2, xEFN_RBD3, or xEFN_RBD4 in isolated mouse RGCs in vitro was tested and shown in FIG. 3D. Treatment with xEFN_RBD3 reduced expression of EphA2 and EphB2. Treatment with xEFN_RBD4 reduced expression of EphA2 and EphB2. Treatment with either xEFN_RBD3 or xEFN_RBD3 reduced expression of EphA2 and EphB2 in both sample groups. Treatment with xEFN_RBD2 did not appear to significantly reduce expression of either EphA2 or EphB2. In FIG. 3E, the effect on growth cone dynamics of 4 days of 1 μM treatment with either xEFN_RBD2, xEFN_RBD3, or xEFN_RBD4 in isolated mouse RGCs in vitro was assessed by immunofluorescent staining for a beta-3 tubulin stain (TUBB3) and a stain for filamentous actin (F-actin). TUBB3 is a marker specific for neuronal microtubules and functions as a neuronal-specific marker. Treatment with xEFN_RBD2 resulted in neuroprotection that was seen by higher expression of TUBB3 than in control in vitro cultures. Treatment with xEFN_RBD3 resulted in neuroprotection that was seen by higher expression of TUBB3 than in control in vitro cultures. Treatment with xEFN_RBD4 resulted in neuroprotection that was seen by higher expression of TUBB3 than in control in vitro cultures. Treatment with xEFN_RBD2, xEFN_RBD3, or xEFN_RBD4 resulted in neuroprotection that was seen by higher expression of TUBB3 than in control in vitro cultures.

### Example 4: xEFN Peptides Desensitize Human Hippocampal Neural Stem Cells (hHNSC) to Growth on Repulsive Ephrin Ligand Substrates

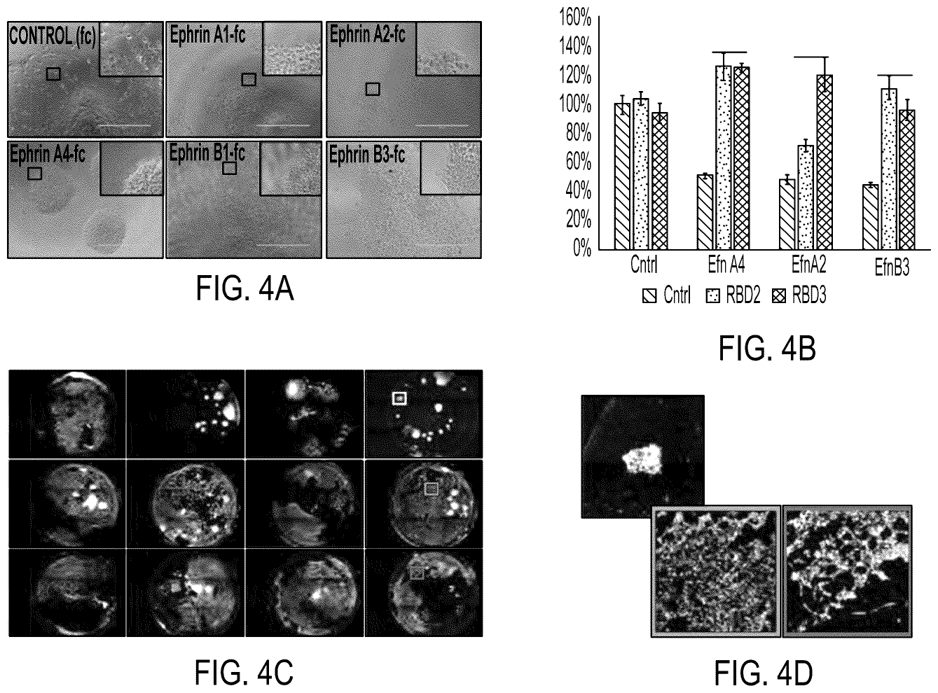

Ephrin ligands are known to represent a repulsive substrate for neuronal growth and neurite extension. An ephrin-ligand substrate test was used to assess the role of xEFN in antagonizing this repulsive stimulus on human hippocampal neural stem cells (hNSC). The results demonstrate that laminin-coated surfaces containing pre-clustered efnA1, A2, A4, B1, or B3 represent highly repulsive substrates to neuronal cell growth (FIG. 4A).

Hippocampal neural stem cells grown on these substrates achieve confluences of 51.16+/−1.55% for efnA4, 48.39+/−3.38% for efnA2, and 44.61+/−1.61% for efnB3 when compared to the laminin-only control confluence (FIG. 4C, p<0.01). Conversely, when supplemented with 500 nM xEFN_RB2 and xEFN_RBD3, hNSC achieve a statistically significant increase in confluence coverage as follows—on efnA4: 126.59+/−9.09 and 125.83+/−2.92%, on efnA2: 71.45+/−3.92 and 120.62+/−10.89%, and on efnB3: 111.03+/−7.79% and 96.51+/−7.15% for RBD2 and RBD3 supplemented cultures over control cultures, respectively (FIG. 4C, p<001).

### Example 5: Intravitreal Injection of xEFN_RBD2 or xEFN_RBD3 Peptide Promote RGC Survival and Axonal Regeneration in an Optic Nerve Crush Model

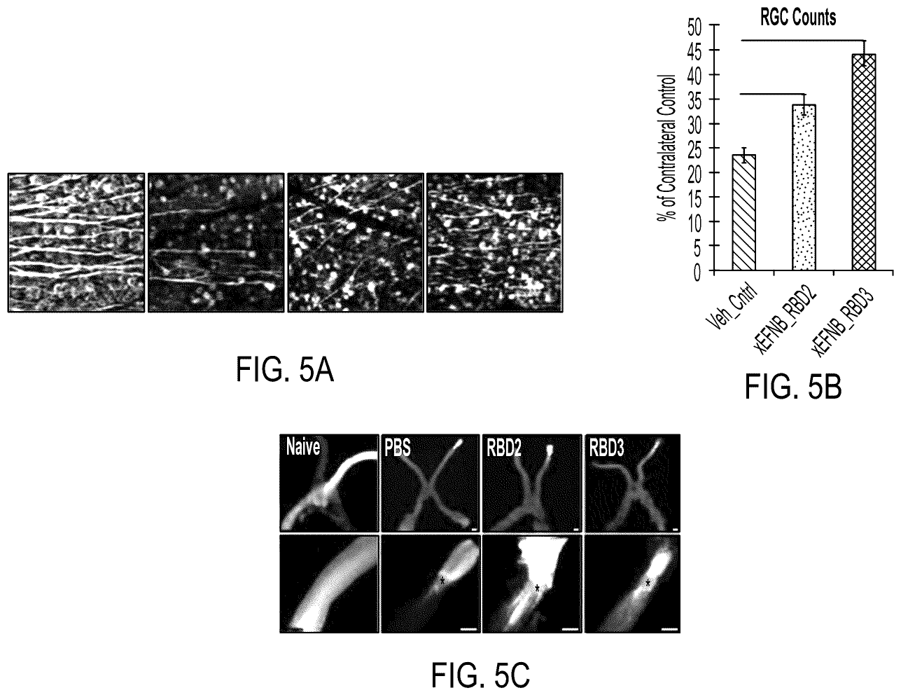

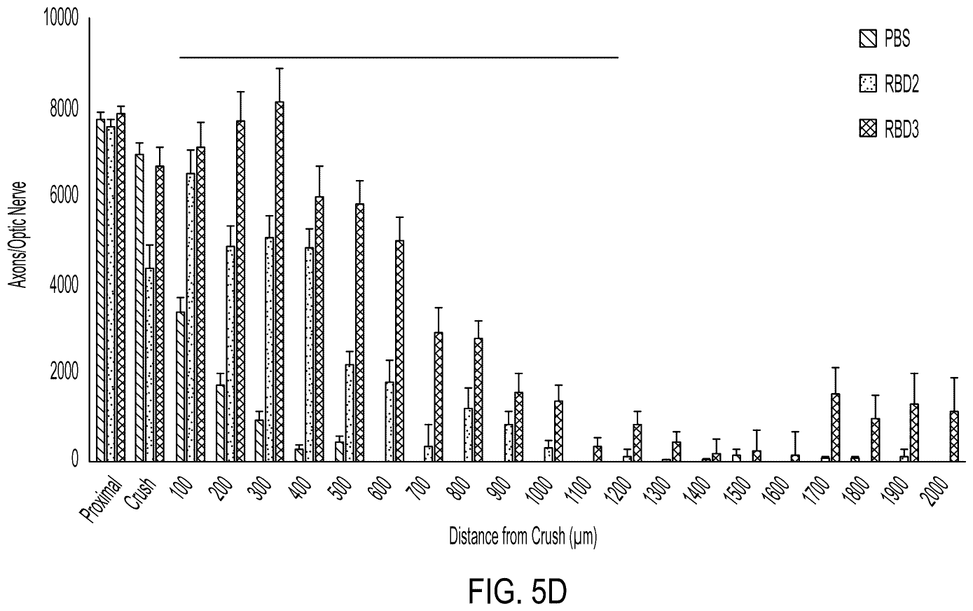

Optic nerve crush (ONC) is a commonly employed model to study retinal neuropathies and glaucoma. Several studies have shown the involvement of different Eph receptors in the pathophysiology of ONC-mediate neurodegeneration. Here, the ONC model was used to study the effect of intravitreally-administered xEFN peptides in ameliorating the neurodegeneration that ensues after injury. The results show that 6 μg of xEFN_RBD2 or xEFN_RBD3 administered twice (immediately after ONC, and 7 days after crush) leads to a significant rescue of the RGC pool over PBS-treated controls (FIG. 5A, B) after 4 weeks of injury. Control (PBS) retinas present with an RGC survival of only 23.50+/−1.48% when compared to the uninjured contralateral retina. Conversely, intravitreal administration of xEFN_RBD2 results in a survival of 33.69+/−1.93% and administration of xEFN_RBD3 in a survival of 44.17+/−2.39% of RGCs at the same time point (FIG. 5B, p<0.01). Immunofluorescent analysis of cholera toxin B (CTB) labeled axon in the optic nerve of the ONC mice revealed a regenerative effect of xEFN peptide treatment in this model (FIG. 5C). In the PBS-treated controls, axonal projections were detected up to 500 μm distal to the crush site (FIG. 5D), while in the xEFN_RBD2 treated animals, axonal projections were detected past 1000 μm distal to the crush site. Yet xEFN_RBD3 administration resulted in the most robust regeneration, with axonal projections being detected up to and beyond the 2000 μm margin of the analysis region of interest (FIG. 5D). A significant increase in regenerating axons was calculated in both xEFN_RBD2 and xEFN_RBD3 treated animals when compared to controls starting at 100 μm past the injury site and throughout the analysis window,

### Example 6: Activation of Several Eph-Receptors of the A- and B-Class Precedes Neuronal Loss and Visual Function Decline in Animal Models of Optic Neuropathy

In order to characterize the activation of Eph receptors in optic neuropathies, two animal models commonly used in the study the disease were employed: the hereditary DBA/2J model, which develop glaucomatous defects of the retina with age, and a traumatic optic neuropathy model (SI-TON) developed in house.

DBA/2J study

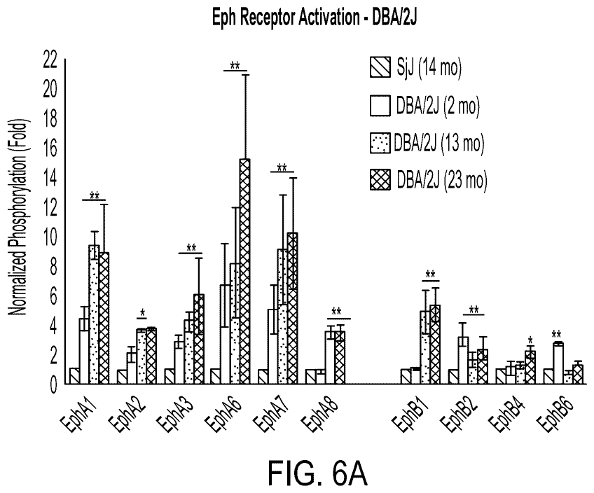

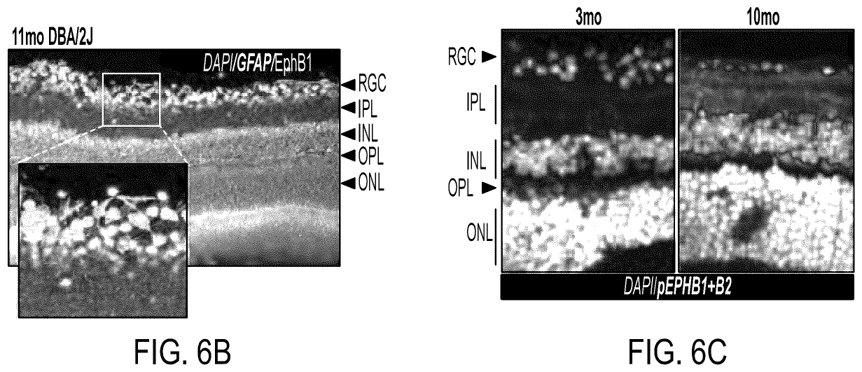

Aging DBA/2J mice develop progressive eye abnormalities that closely mimic human hereditary glaucoma, with females presenting visual dysfunction earlier than males. Regardless, by 9 months of age, both sexes exhibit elevated IOP and loss of RGCs in the retina. The Proteome Profiler Human Phospho-RTK Array Kit (RnD Systems) was used to perform relative quantitation of phosphorylated Eph receptors in an age dependent manner in male DBA/2J animals as compared to their genetic control SjJ (FIG. 6A). The results show a consistent and reproducible age-dependent increase in the activation of several Eph receptors in the DBA/2J animals, with 9/10 Eph receptors probed being significantly increased in phosphorylation state at 23 months of age. Interestingly, however, several Eph receptors in the assay (EphA1, EphA3, EphA6, EphA7, EphB1, EphB2, and EphB6) showed a statistically significant increase in phosphorylation by the 2nd month of age, a time point in which clinical manifestations of visual dysfunction are still not detectable in the male DBA/2J (FIG. 6A) mouse.

Immunohistochemical images show the presence of the EphB1 receptor exclusively localized to the RGC layer in the 11-month DBA/2J animals (FIG. 6B). In concordance with the RTK profiler assay, immunohistochemical imaging of the DBA/2j retina shows an age dependent activation of the EphB1+B2 receptors in the inner retinal layers (FIG. 6C).

### Activation of Eph Receptors in Early Traumatic Optic Neuropathy (TON).

Traumatic optic neuropathy is an orphan disease associated with closed head trauma and TBI that leads to progressive RGC loss and complete blindness by 2 weeks post-trauma. In order to evaluate the involvement of Eph-receptors in the pathology, immunohistochemical evaluation of retinas of injured animals acutely (4 and 48 hours) after trauma to the optic nerve was analyzed. The results show that receptors EphA2+A3+A4, and B1+B2 (Abcam antibodies #ab62256 and ab61791 respectively) become phosphorylated at this acute time point in the entire inner plexiform layer of the retinas, and that activation of these Eph receptors on the RGC somas is closely associated with sites of interactions between RGCs and astrocytes, as well as to Muller Glia within the inner plexiform layer. At these time points, the RGC pool is still indistinguishable from uninjured controls in number and dendritic arbor complexity. Yet the RGC soma and neurite projections become increasingly marked with activated Eph receptor moieties during this early period of neuropathic initiation.

### Example 7: Design, Production, and Validation of a Pan-Eph-Receptor Knockdown System Using Adeno-Associated Virus (AAV2)-Viral Delivery of shRNA Constructs

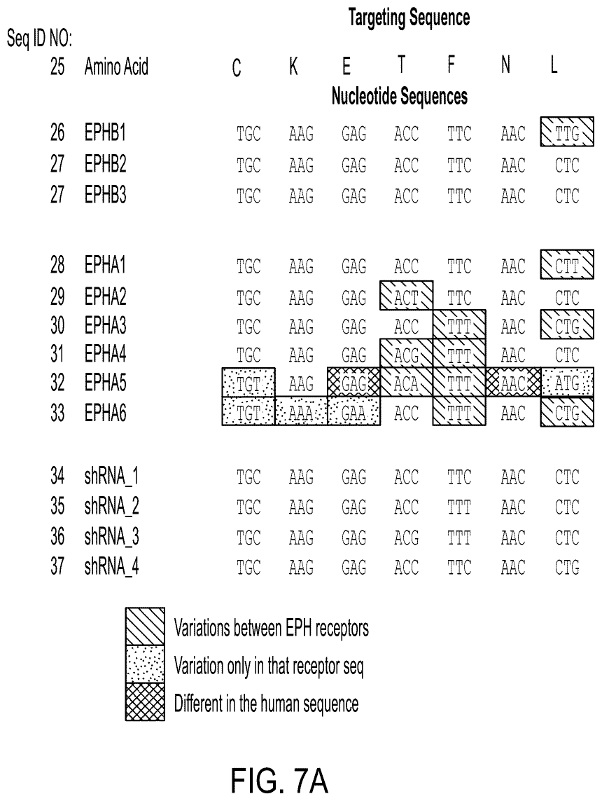

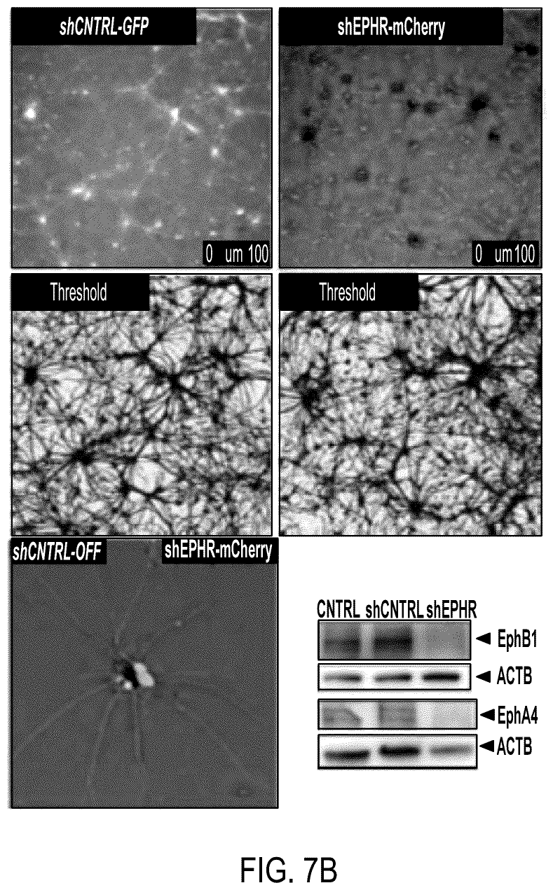

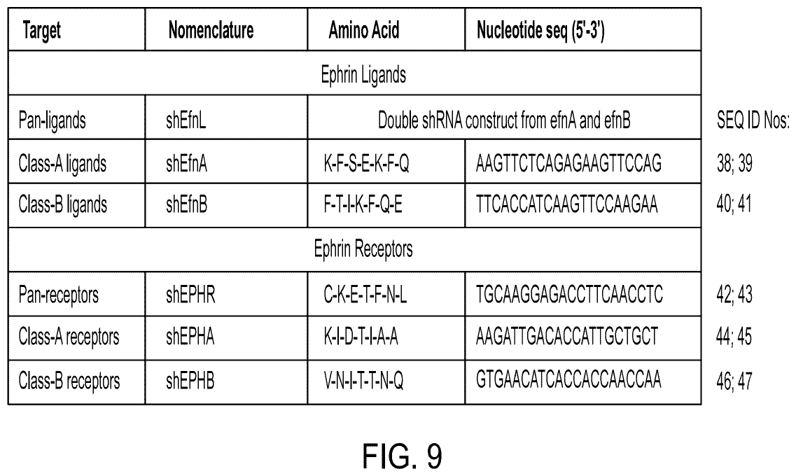

There are many technical challenges to modulating Eph-receptor signaling in the CNS as outlined above. Most importantly, the sheer number of EphA and EphB receptors involved in retinotopy, the compensatory and redundant biological roles described for them, and the involvement of several Eph-receptors (of both classes) in any given neuropathic disease. The inventors have identified amino acid motifs and nucleotide sequences that allow for the shRNA targeting of class A (shEPHA), class B (shEPHB), and general Eph receptors (shEPHR) through a gene therapy approach. The use of a library of shRNAs targeting the amino acid sequence C-K-E-T-F-N-L which is present in all Eph receptors (FIG. 7A) was evaluated. These sequences were cloned into a binary Adeno-Associated Virus serotype 2 packaging system (AAV2-shRNA) and used for in vitro and in vivo gene knockdowns. Transduction of isolated mouse RGCs in vitro with the shEPHR viral particles showed no cytotoxicity of the targeting sequences and demonstrated an increase in dendritic network density arising from these isolated neurons when compared to a sh-Control plasmid (FIG. 7B). Similarly, co-transduction of RGCs with shCNTRL and shEPHR particles showed that neurites sprouting from shEPHR expressing cells remained patent longer in culture than those transduced with the shCNTRL sequences (FIG. 7B). Western blot analysis of transduced cells showed a significant knockdown of the EphB1 and EphA4 receptor in the shEPHR group (FIG. 7B). FIG. 9 lists the nucleotide sequences for shRNA constructs for Ephrin ligands and Eph receptors in a table.

Finally, viral packaging was scaled up to produce titers of at least 1×10¹² TIU/ml and used to transduce RGC in vivo through intravitreal injections in C57BL/6J wildtype mice. Fluorescent and immunohistochemical imaging shows a robust efficiency of transduction in the RGC layer of retinas at 4 weeks post-injection with no toxic effects seen from either targeting vectors.

### Example 8: Spatial Transcriptomic Map of the Normal and Neuropathic Postnatal Mouse Retina

To characterize the spatiotemporal expression and presentation of all Eph receptors within the postnatal retina and to evaluate their relationship to the regional loss of retinal neurons in animal models of optic neuropathy, transcriptomics assays are used. Using spatial transcriptomics assay, fresh frozen sections are mounted onto a specialized microscope slide in one of 8 capture areas. Each area contains 50,000 pre-adsorbed probes of sequencing genetic barcodes and unique molecular identifier (UMI). Probe spots are 50 μm in diameter and are spaced 100 μm center-to-center apart from each other. It is proposed to mount fresh frozen retinas in such a way that the inner retina (RGC side) lays onto the barcoded probes for the capture of RGC-specific regional transcriptomic information. Retinas from wildtype C57BL/6J mice are analyzed at 1, 6, 12, and 24 months of age (2 each for age-related transcriptomic changes)). Six-month old C57BL/6J animals are used, induce traumatic optic neuropathy using the SI-TON model, and analyze the retinas for regional transcriptomic changes at 24 h, 48 h, 7 days, and 14 days post SI-TON (2 each for injury-time course related changes), using the naïve 6-month old retina for control. Finally, the hereditary glaucoma model DBA/2J model and age- and sex-matched Gpnmb+ controls are used. Male DBA/2J and Gpnmb+ animals are analyzed at 1, 6, 10, and 15 months of age to capture the age-related transcriptomic changes in this animal model from healthy to fully-developed glaucomatous defects. Eph receptors that are retained at the expression level within the postnatal mouse retina and exploit the full capabilities of this platform by analyzing the age-related (hereditary), and time course-associated (trauma) transcriptomic changes in the full transcriptome through neuropathy are explored but generate this information at the highest regional resolution ever achieved.

### Example 9: Regional Proteomic Analysis of Eph-receptor Expression and Phosphorylation in the Normal and Neuropathic Postnatal Retina

One of the premises of these studies is that Eph-receptors present in the postnatal retina become activated in early neuropathic disease. To test this hypothesis, it is proposed to study the proteomic compartment of the retina in an age- and injury time-related assay. The same animal cohorts and time points are used for each animal strain, with 3 animals per sampling point. Laser-capture microdissection using the Zeiss PALM microbeam laser microdissection system are used to partition the retina into 12 different regional segments. Retinal samples from the Dorsal (D), Ventral (V), Nasal (N), and Temporal (T) quadrants are taken and further microdissecting these radially into central (c), middle (m), and peripheral (p) retinas, thus generating the twelve samples per retina: Dc, Dm, Dp, Vc, Vm, Vp, Nc, Nm, Np, Tc, Tm, and Tp. These samples are analyzed using the Jess protein simple western capillary platform that allows for the analysis of proteomic samples in a 3 μL volume, making this approach feasible. While not at the resolution of spatial transcriptomics, this microdissection of the retina gives the highest resolution proteomic analysis of the Eph/ephrin signaling pathway in the adult mammalian retina available. All Eph-receptors and phosphorylated members for which there are commercially available antibodies are analyzed.

### Morphometric and Functional Assessment of Visual System Prospectively in Optic Neuropathies

There must exist a molecular reason for the consistent pattern of neuronal loss in neuropathic diseases of the retina. This entire aim is dedicated to elucidating this mechanism by focusing primarily on the developmental cue most responsible for axonal organization in the visual system. This study characterizes morphological changes in the retina as it progresses through neuropathy. A Heidelberg Spectralis OCT system is used to generate morphometric heatmaps of the inner retinal thickness (RGC through IPL) and further perform functional electrophysiology recordings via pattern electroretinograms (PERG) to assess morphology-function correlations. Both the DBA/2J system, as well as the SI-TON model is used at the same time points in order to be able to correlate morphological and functional changes in the retina to regional transcriptomic and proteomic differential expression through progression. 10 male DBA/2J and 10 Gpnmb+ male control animals, as well as 10 C57BL/6J naïve, and 10 C57BL/6J SI-TON injured animals are used prospectively.

It is expected that several Eph receptors are dysregulated at the gene expression level in progressive glaucomatous defects in the DBA/2J animal. Based on immunohistochemistry findings, it is expected to see this in the traumatic optic neuropathy model as well

### Example 10: Evaluate the Dynamics of Retinal Glia-Neuron Interactions and the Role of Eph/Ephrin Signaling in RGC Synaptic Instability and Retraction

Dendritic spines and synapses, as well as general neurite projections are not static structures anywhere in the nervous system, they are highly dynamic systems that continuously undergo remodeling in response to many different stimuli. Among the major contributors to the systems' dynamism is the interplay between neurons and surrounding cellular components like glial cells. In the CNS, glia-neuron interactions via Eph/ephrin signaling at the synaptic interface have been shown to mediate processes such as long-term potentiation (LTP), as well as trigger degenerative synaptic loss in disease states. In the retina, there are several glial components that can play this critical role. Astrocyte, Muller Glia, and microglia all reside within the layers of the retina and interact with RGC neuronal processes and synapses within its plexiform layers. Yet no one has shown how this interaction and Eph/ephrin signaling may play a role in shaping and maintaining RGC arbors and receptive fields, despite Eph/ephrin signaling known role in retinotopic development. Furthermore, with the recent evidence of the Eph/ephrin signaling cascade dysregulation in optic neuropathies, it is crucial that further elucidation is obtained for how gliaRGC interactions mediate or counteract the initiation and progression of neurodegeneration in the visual system. This study focuses on analyzing the glia-RGC interplay through the Eph/ephrin signaling cascades as it relates to synaptic plasticity, strength, and remodeling. Given their highly labile nature, RGC primary cultures are not amenable to much genetic manipulation.

### RGC and Retinal Glia Co-Culture Systems

The dynamics of glia-RGC interaction in promoting or inhibiting RGC neurite extensions are explored, establishing dendritic arbor complexity, and axonal polarization. This is evaluated in the context of Eph/ephrin signaling modulation by genetically manipulating the glial component of the system to knock-down specific ephrin ligand and receptor classes. This involves co-culture of isolated retinal glia (Muller Glia, Astrocyte, or microglia) with GFP-labeled RGCs isolated by immunopanning from a Rosa26-eGFP mouse strain (Tg(Gt(ROSA)26Sor-EGFP)IIAble/J). Neurite density analysis is performed over a 7-day period (with timelapse microscopy and endpoint Sholl analysis), as well as confocal imaging of dendritic spines probed for Eph receptors and ephrin ligands (as shown in FIGS. 6A-6C) at days 0, 3, 5, and 7 of co-culture.

This baseline system is perturbed by repeating the experimental setup while using glial cultures in which ephrin-ligands or receptors have been knocked down using one of the shRNA constructs against class-specific and pansequences that have been curated at the nucleotide level for homology.

Assessment of RGC Synaptic Strength Upon Stimulation with Pre-Clustered Ephrin Ligands

Stimulation of RGCs with some pre-clustered ephrin ligands (like efnA5) is known to induce axonal growth cone collapse through Eph-receptor activation, yet no one has reported on the full gamut of ephrin ligands with respect to their repulsive effect on neuronal projections.

Still much less is known about the ephrin ligands that can trigger synaptic spine retraction. In Alzheimer's disease, the glia-bound efnA3 ligand has been associated with this neurodegenerative process. To investigate, an entire library of ephrin ligands (RnD Systems ephrin ligand libraries) is evaluated with respect to synaptic stability in RGCs. Mouse pup (p0-p5) RGCs are obtained through immunopanning and set them in cell culture for 4 days, at which timepoint a dense network of neurite and axonal connections has been achieved. At this time point, RGCs are stimulated by addition of 2 μg of pre-clustered ephrin ligand for a period of 6 hours (efnA1, A2, A3, A4, A5, A6, B1, B2, and B3 are used). The ephrin ligand recombinant library available include a human IgG1 Fc domain (Pro100-Lys330) on their c-terminus that allows for pre-clustering of the ligands with anti-human IgG antibody for 30 minutes prior to RGC stimulation. Controls include anti-human IgG antibody alone, as well as non-clustered ephrin ligands. Protein lysates to assess differential levels of synaptic strength-associated proteins such as NMDA (GluN1, GluN2) and AMPA (GluA1, GluA2) receptors, and synaptic scaffolding proteins DLG4 (aka PSD95), DLG2, and synaptophysin (SYP) are used. The experiment is also be performed on RGC that have been transduced for 4 days with the shEPHR AAV2 constructs to elucidate whether the specific ligands act through EphA, EphB, or both classes of Eph receptor members.

### Proteomic Analysis of Inner Versus Outer Retinal Expression of Eph Receptors and Ephrin Ligand Moieties

This method focuses on the third axis, the proximal-distal axis by looking in retinal cross-sections for changes in Eph receptor expression and activation. The preliminary studies demonstrate that the aberrant activation of Eph receptors in neuropathic states occurs within the inner retinal layers. This observation is very important to the identification of retinal phenotypes involved in this signaling because the soma of specific retinal cells reside within one of the nuclear layers of the retina. For example, the absence of the signal in the outer plexiform and nuclear layers rules out the involvement of bipolar and photoreceptor cells, while the signal in the inner nuclear and plexiform layers does not allow one to rule out amacrine and horizontal cells and can involve all of the glial cell compartments evaluated herein. Retinal cross sections of the DBA/2J and Gpnmb+ controls, as well as naïve and SITON injured C57BL/6J retinas are used. The Zeiss PALM microbeam laser microdissection system is used to separate inner and outer retinal regions. Given the amount of sample required, it is proposed to use at least 20 cross-sections of retina per sample (from 5 animals per sample) and requires the Jess protein simple capillary-based proteomics platform to achieve discernible readouts.

### Super-Resolution Microscopy of Synaptic-Localized Eph Ephrin Molecules in the Inner Plexiform Layers of the Healthy and Neuropathic Retina

The role of glia in modulating synaptic strength and remodeling is undeniable and has been shown in many different systems. Recently, the involvement of the Eph/ephrin system in this synaptic modulation has been demonstrated in the brain. Since Eph/ephrin signaling is central to retinotopic map and RGC receptive field establishment, this method looks into the retinal glia-RGC interplay at the synaptic interface within the RGC dendritic arbors during neuropathy. Two proposed optic neuropathy animal models, the DBA/2J and SI-TON models are used, at the ages and time points outlined above. Eph-receptor clustering in RGC dendritic spines is examined (identified by counterstaining with TUBB3, or MAP2), ephrin ligand expression on glial membranes (as identified by counterstaining with GFAP (astrocytes), GLUL (Muller Glia), or Cd11b (microglia)), and perform proximity measurements between these markers through the use of a Zeiss ELYRA stochastic optical reconstruction microscope (STORM).

It is hypothesized that glial membranes modulate synaptic stability and remodeling in the RGC dendritic spine, and expect that upon reactivity, this modulation leads to detrimental effects on RGC synaptic connectivity.

### Example 11: In Vivo Delivery of AAV2-shRNA Knockdown of EphAs, EphBs, and General EphRs in Animal Models of Optic Neuropathies

Researchers have shown the beneficial regenerative effects in the visual system upon the deletion of specific Eph receptors in RGCs, or ephrin ligands in glial compartments. Yet, a growing body of evidence suggests that multiple Eph family members are associated with the onset and progression of neuropathic disease of the retina. This is not in itself surprising given the involvement of almost all Eph-receptors in the development of retinotopic projections.

Curated shRNA libraries and AAV2 delivery system are used for a pan-knockdown of Eph receptors (shEphR), as well as to specifically suppress A- and B-class Eph receptors (shEphA and shEphB, respectively) to evaluate their contributions to neuropathic initiation and progression of visual dysfunction.

### Evaluate the Effect of Knocking Down EphAs, EphBs, and Pan-Eph Receptors on Retinal Ganglion Cell (RGC) Dendritic Arbor Morphology

Synaptic instability, retraction, and ultimate dendritic and neuronal loss are the classic manifestation of neuropathic progression. It is hypothesized that this process can be initiated by the activation of repulsive Eph forward signaling on the neuronal membrane. Yet, no one has ever analyzed the contribution of the different Eph receptor classes in this pathologic mechanism and given that almost all Eph receptors are involved in retinotopic development, it is important to assess which may play a stronger role in promoting the neuropathic degeneration of the retina.

This method evaluates the use of the targeting vectors against class-specific and pan-Eph-receptors in preserving dendritic spines, and arbor morphology even in the context of neuropathic disease. Evaluating individual RGC dendritic arbor morphology requires the sparse or selective labeling of individual RGCs in order to be able to accurately capture and prospectively imaging the arbor morphology. To accomplish this, 4-week old C57BL/6J and DBA/2J animals (n=25 each) undergo retrograde labeling of RGC by application of the fluoro-gold probe on the surface of the superior colliculus (SC). Briefly, animals are anesthetized and the skull is surgically exposed by a single incision. The bregma is localized, and with the use of calipers, two spots are marked above the SC, which is 2.9 mm caudally, and 0.5 mm laterally of bregma, on either side. Using a microtip drill, a small hole is delicately bored at those spots through the cranial bone, without injuring the surface of the brain. Then, a 36G needle is lowered using a micromanipulator to a depth of 1.6 mm form the surface of the brain, and 1 μL of the fluoro-gold probe is injected at a rate of 0.2 μL/min with the assistance of a microinjection pump and Hamilton syringe. The cranial openings are then closed using sterile surgical foam, and the skin above the skull is closed and the mouse is allowed to recover from anesthesia. Two weeks after SC labeling, animals undergo fluorescent imaging of labeled RGCs in the Spectralis laser-scanning confocal Ophthalmoscope to acquire baseline arbor morphology prior to the onset of neuropathy. By careful positioning of the animals on the acquisition stage, this technique can be used to image the same set of RGCs and arbors over time. Following baseline imaging, animals receive an intravitreal injection of viral particles with the shEphA (n=5 ea), shEphB (n=5 ea), shEphR (n=5 ea), or shCNTRL (n=5 ea) targeting plasmids (>1012 TIU/ml in 1.5 μL volume). A subset of animals (n=5 ea) received no injection and are used as neuropathic model controls. At 8-weeks of age (4-weeks post-retrograde labeling), all C57BL/6J animals (n=25) undergo sonication-induced traumatic optic neuropathy, SI-TON (140), and their RGC morphology is assessed again at 1, 2, 7, 14, and 21 days post-injury. DBA/2J animals (n=25) undergo prospective morphometric measurements of the RGC pool at 2, 6, 9, 12, 15, and 20 months of age. Fluorescent RGC images are then thresholded and analyzed using Sholl Analysis. Dendritic arbor morphology and complexity changes are quantified for each individual RGC using a complexity score consisting of the compounded values of the ‘intersecting radii’ and ‘number of branching points’ parameters produced by the Sholl analysis, normalized to baseline soma diameter, such that: arbor complexity=(#of branching points x intersecting radii distance)/(diameter of soma at t=0). Since the analysis is to be performed on individual RGC arbors, this quantitation is irrespective of the subtype of RGC labeled, which can then be further used for sub-cluster analyses of the effects of Eph receptor knockdowns in the subpopulations of RGC captured by the screening. At the end of each experimental cohort, animals are euthanized, and the dissected retinas are processed as follows: 4 retinas lysed in RIPA buffer for Western Blot quantification of Eph-receptor knockdown, 4 retinas for cross-section imaging of RGC dendritic arbor depth into inner plexiform layer by MAP2 staining, and 2 retinas for flatmount tilescan high resolution imaging of retinal axonal bundle morphology using the LEICA SP8 Laser Scanning Confocal microscope.

### Assess Visual Function Progression in Animal Models of Optic Neuropathy Following EphA, EphB, or Pan-Eph Receptor Knockdown in RGCs.

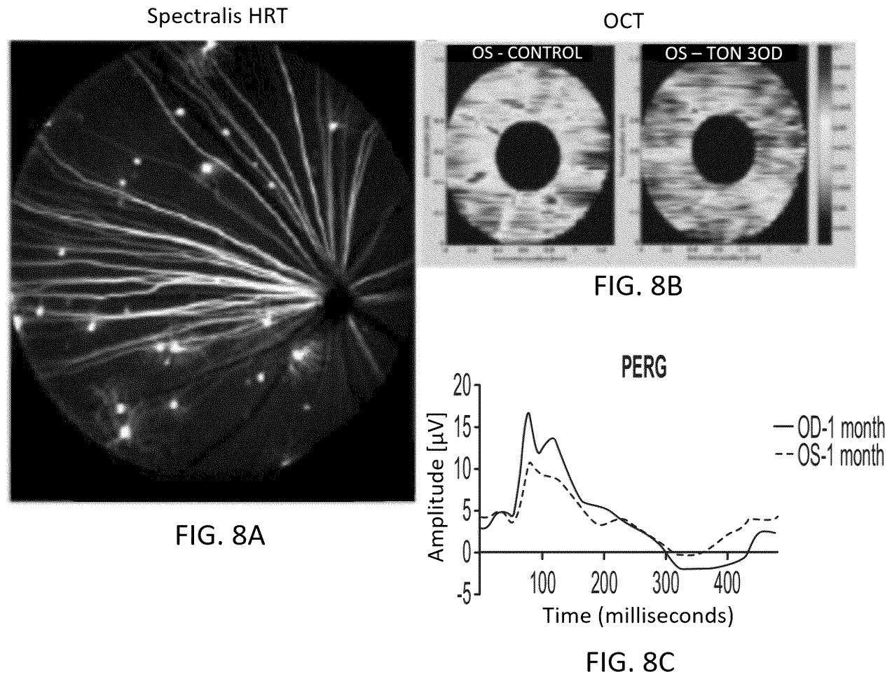

Maintaining or restoring visual function is the ultimate goal in ophthalmology. This method evaluates visual function prospectively upon the modulation of EphAs, EphBs, or EphR. This avoids the confounding variable of what superior colliculus labeling of RGCs may do to visual function tests, and solely focus on visual function parameter testing. To accomplish this method, the two optic neuropathy models described above, the DBA/2J and SI-TON models, are used. At 4-weeks of age, C57BL/6J and DBA/2J animals (n=40 each) undergo baseline visual function recording via PERG and visual system morphology with OCT using the Spectralis laser scanning ophthalmoscope with OCT. Animals then immediately receive an intravitreal injection of AAV2 viral particles with the shEphA-mCherry (n=10 ea), shEphB-mCherry (n=10 ea), shEphR-mCherry (n=10 ea), or shCNTRL-GFP (n=10 ea) targeting vectors (>1012 TI U/ml in 1.5 μL volume). At 8-weeks of age (4-weeks post-AAV2 injection), all C57BL/6J animals (n=40) undergo sonication-induced traumatic optic neuropathy, SI-TON. Animals are allowed to progress through neurodegeneration for 2, 7, 14, and 21 days, at which points animals of each targeting cohort undergo PERG and OCT measurements to assess visual function progression (FIGS. 8A-8C). For the DBA/2J animals, visual function recording time points are 6, 9, 12, and 15 months of age. At the end of the experimental period, excised retinas for both animal models are probed for TUBB3 in order to calculate RGC survival.

It is hypothesized that the global knockdown of Eph-receptors to have a significant effect on the disease course and that this effect is beneficial given the known repulsive role for Eph receptors to RGC projections, as well as from data and previous reports, showing that knockdown of EphB1 and EphA4 separately confer regenerative outcomes in optic neuropathies. It is also expected that contributions from both the A- and B-type receptors are similar in potency because of the high promiscuity and redundancy built into the Eph/ephrin signaling pathway.

### Example 12: Treating Cells with xEFN_RBD2 or xEFN_RBD3

Previous examples have demonstrated that treatment of cells with xEFN_RBD2 or xEFN_RBD3 significantly increased neurite sprouting, axonal extension, and dendritic arbor complexity in isolated retinal ganglion cells. (FIGS. 2A-2B.) Further, this dendritic sprouting and axonal extension effect exerted by the xEFN peptides was mediated through antagonism of Eph-receptor phosphorylation and its effect on downstream cytoskeletal signaling dynamics. (FIGS. 3A-3C.) This Eph-receptor antagonism resulted in a significant reduction in the number of collapsed versus outgrowing growth cones in RGC neurites in culture (FIG. 3D), as well as a desensitization of neuronal membranes to growth on repulsive ephrin ligand-coated surfaces (FIGS. 4A-4D.)

In-vivo studies showed that intravitreal administration of peptides xEFN_RBD2 and xEFN_EBD3 resulted in significant RGC survival and axonal regeneration in an acute model of severe optic nerve injury, the optic nerve crush (ONC) at 4 weeks post trauma. (FIGS. 5A-5D.

### Example 13: Assessing the Effect of xEFN Peptide Administration on the Visual Function of Mice

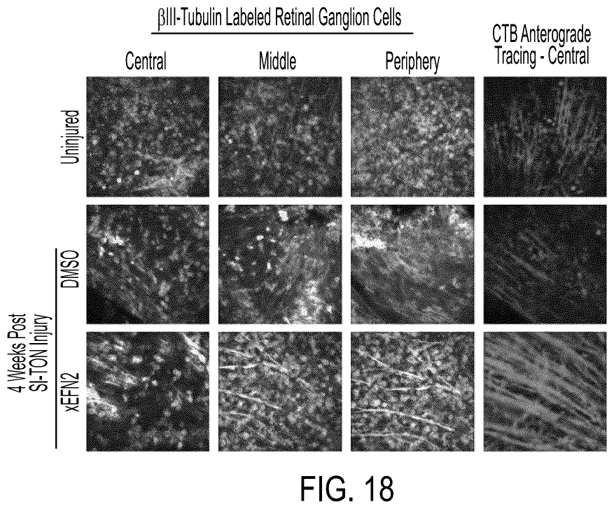

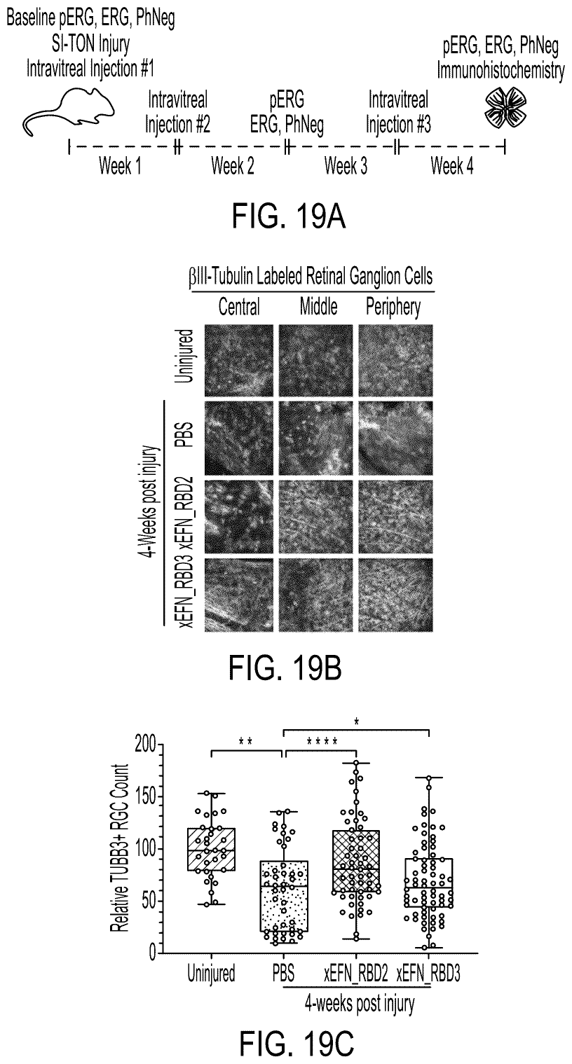

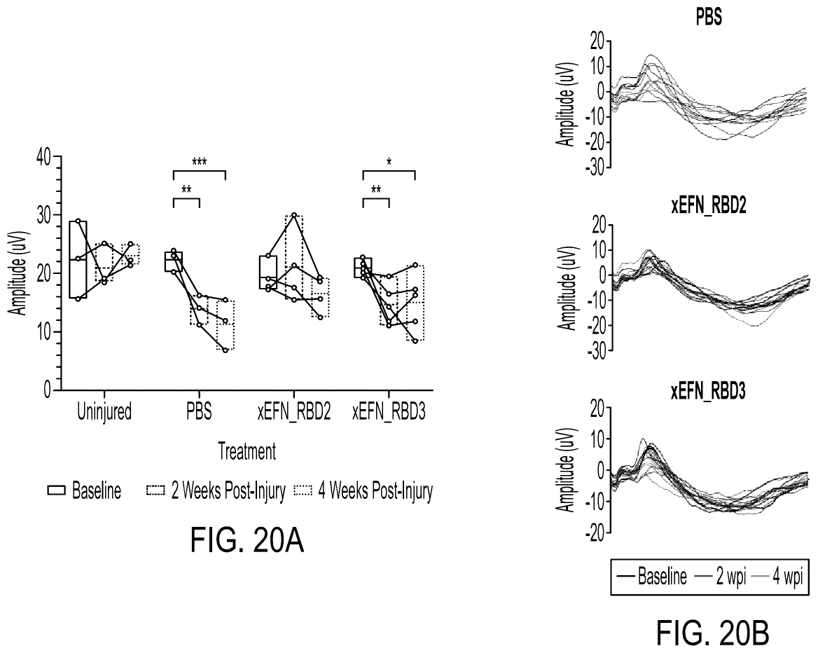

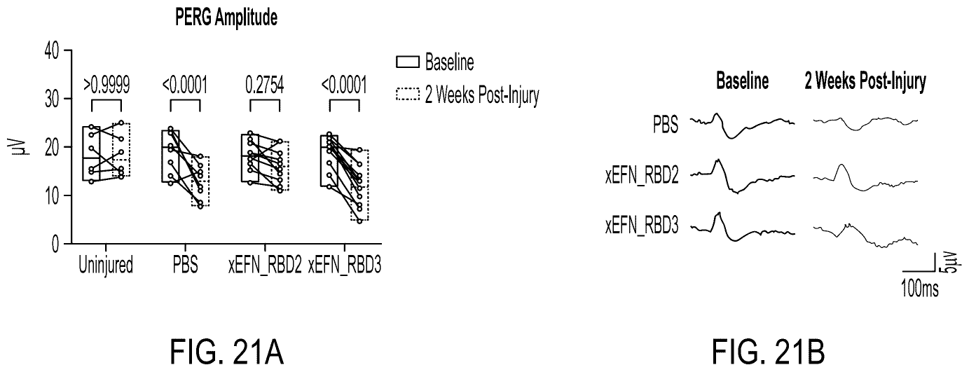

In this example, the effect of xEFN peptide administration on the visual function of mice in an animal model that more closely resembled the human manifestation and progression of visual dysfunction following the onset of optic neuropathy was assessed. An ultrasonic shockwave model of the optic nerve injury and neuropathic induction helped to demonstrate that administration of xEFN peptides xEFN_RBD2 and xEFN_RBD3 conferred a significant survival effect on RGCs 4 weeks post injury when compared to vehicle treated controls in this model (FIGS. 19A-19C). Visual function was measured at 2 and 4 weeks post optic nerve injury in all groups using pattern electroretinogram (PERG) to measure RGC function, as well as flash electroretinogram (ERG) to assess rod and cone functionality. The data showed that administration of xEFN-RBD2 peptide preserved functional vision in these animals with no decrease in PERG amplitude seen over baseline recordings (FIG. 21A). The data further showed that the visual function of animals treated with xEFN_RBD2 was maintained throughout the 4 week assessment timeframe (FIG. 20A) and that the electrophysiological function of RGCs in these animals was comparable to that of naïve (uninjured) age-marched animals (FIG. 20A).

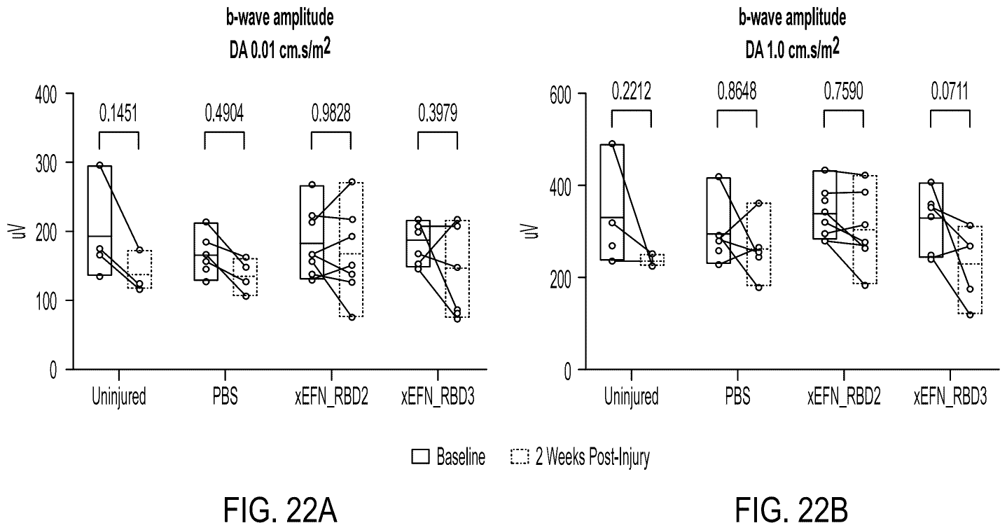

While the visual function of animals treated with xEFN_RBD3 peptide was observed to decline over a 2 week period (FIG. 21A), at the 4 week assessment, the visual response in these animals was improved (FIG. 20A) and was significantly better than that of vehicle (PBS) treated animals (FIG. 21A). Finally, flash ERG recordings in these animals showed that the administration of xEFN peptides was non-toxic and did not affect outer retinal function as was demonstrated by the non-significant changes in b-wave response amplitude in dark adapted (DA) animals for rod (FIG. 22A) and rod and cone (FIG. 22B) functional assessment.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### Example 14: Measurement of Binding Kinetics of xEFN Peptides to Eph Receptors

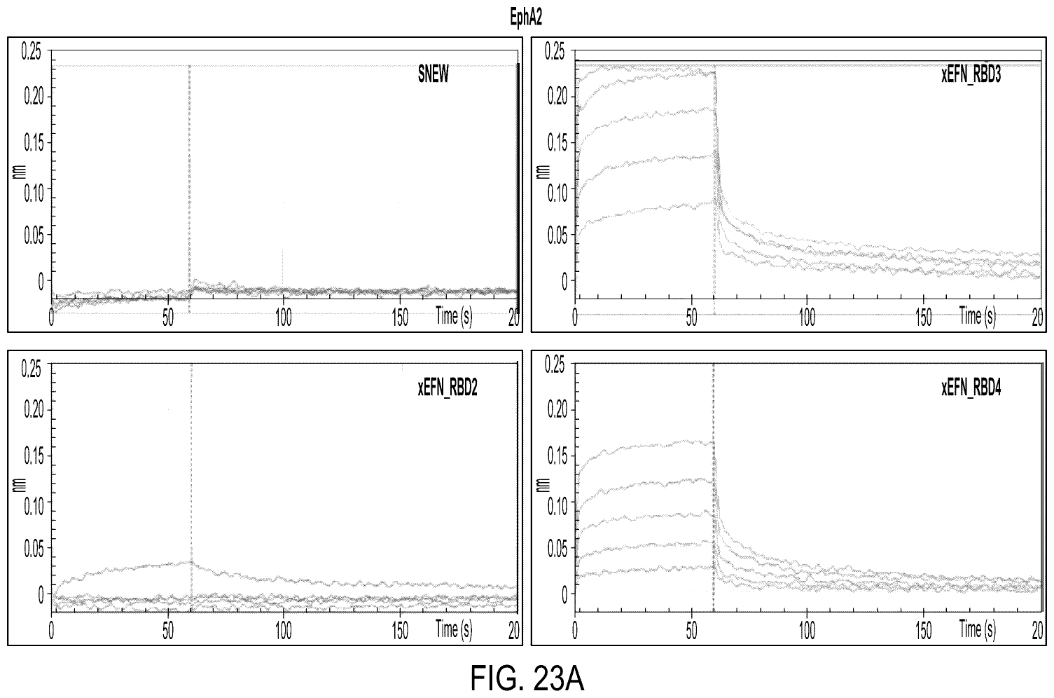

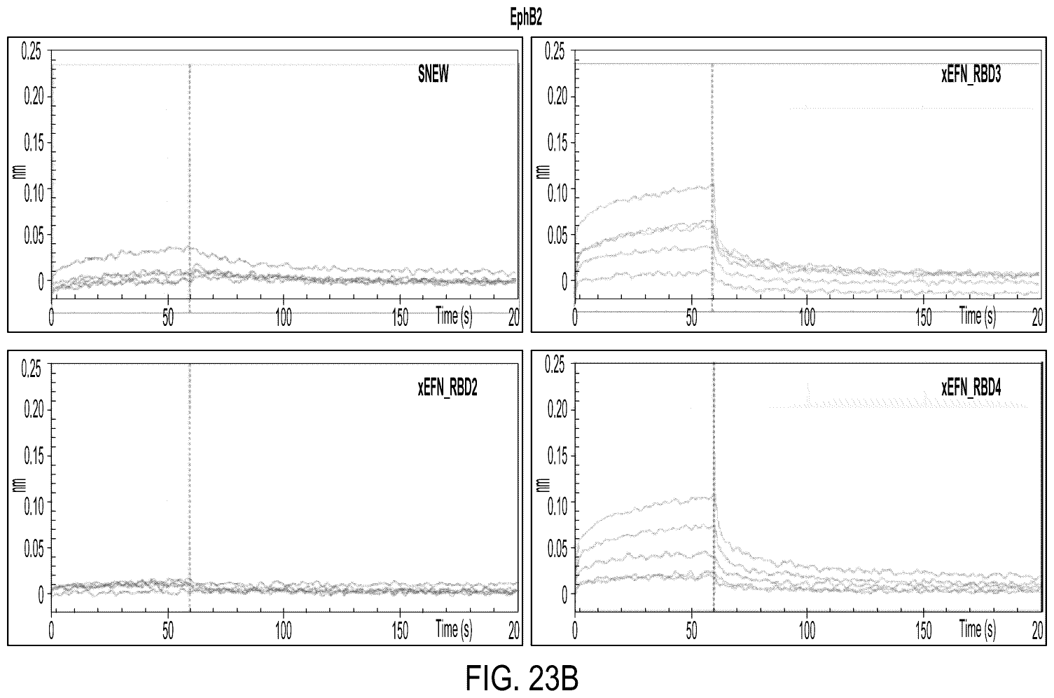

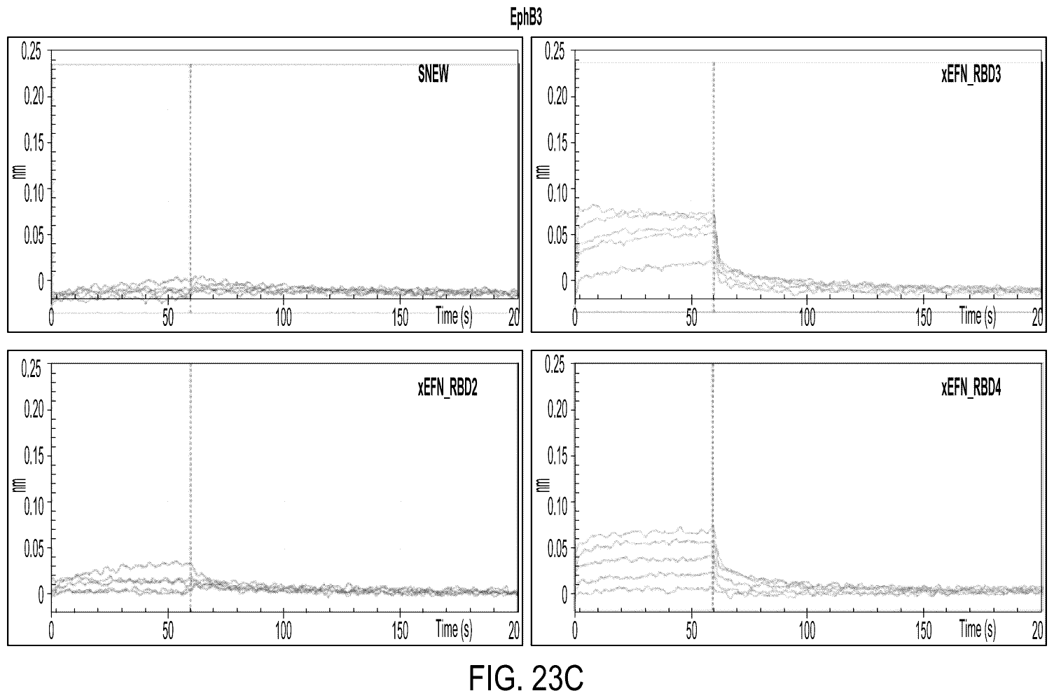

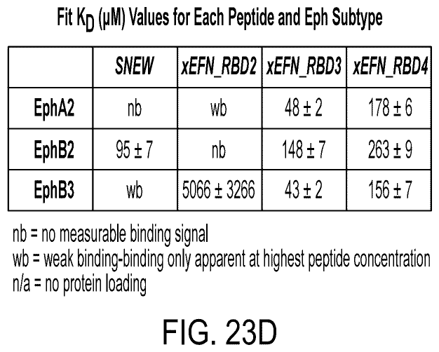

In this example, the binding kinetics of xEFN_RBD2, xEFN_RBD3, and xEFN_RBD4 were assayed for binding and dissociation from EphA2, EphB2, and EphB3. Additionally, a known EphB2 targeting peptide sequence (SNEW) was tested concurrently. SNEW (with peptide sequence SNEWIQPRLPQH) has been shown to bind with high selectivity and moderate affinity for EphB2 and is believed to inhibit Eph-ephrin interactions by competing with various ephrin ligands for the EphB2 high-affinity pocket (MaB et al. Investigation of the interactions between the EphB2 receptor and SNEW peptide variants. Growth Factors. 2014 Dec; 32(6):236-46). SNEW does not share significant amino acid sequence alignment or homology with peptide ligands described herein. SNEW was previously described and had its three-dimensional structure in complex with the EphB2 receptor in Chrencik J E et al. Three-dimensional structure of the EphB2 receptor in complex with an antagonistic peptide reveals a novel mode of inhibition. J Biol Chem. 2007 Dec. 14; 282(50):36505-13. Label-free Biolayer Interferometry (BLI) was used to determine binding kinetics for the assayed peptides. Binding kinetics were measured using a ForteBio Octet TX biolayer interferometry instrument (Molecular Devices ForteBio LLC, Fremont, CA) according to manufacturer instructions. Binding response was recorded and specific signal was normalized by subtracting background control from the dataset. Data analysis was performed with Octet Data Analysis HT 10.0 software. BLI was used to assay kinetic binding constants (Kₐ, K_{d}) and equilibrium binding constants (affinity, Kₐ=1/K_{d}). Response was measured as a nm shift in the interference pattern and is proportional to the number of molecules bound to the surface of the biosensor. Response was plotted in sensorgrams for the 4 test peptides with binding kinetics toward 3 Eph receptors: FIG. 23A for EphA2, FIG. 23B for EphB2, and FIG. 23C for EphB3. Response was plotted on the y-axis and time was plotted on the x-axis. Fitted dissociation constant (Kₐ) expressed in μM values for each assayed peptide for each receptor subtype were collected and are listed in FIG. 23D. In this assay, SNEW exhibited strong binding for EphB2 and weak binding (wb) or no measurable binding (nb) for EphB3 and EphA2 respectively. xEFN_RBD2 displayed moderate binding for EphB3, wb for EphA2 and nb for EphB2. xEFN-RBD3 displayed strong binding for EphA2, EphB2, and EphB3. xEFN-RBD4 displayed strong binding for EphA2, EphB2, and EphB3, though at slightly higher K_{d} levels than xEFN-RBD3 for each receptor. In this assayed, xEFN-RBD3 and xEFN-RBD4 were determined to each bind strongly to EphA2, EphB2, and EphB3 indicating that both peptides are capable of forming specific binding complexes with each of EphA2, EphB2, or EphB3. xEFN-RBD3 was determined to bind more strongly that xEFN-RBD4 to each of EphA2, EphB2, and EphB3. xEFN-RBD2 was determined to bind moderately to EphB3 but not significantly to other tested Eph receptors. This binding by xEFN-RBD3 and xEFN-RBD4 across Eph receptor subtypes indicates that these peptides successfully target various Eph receptors including EphA2, EphB2, and EphB3. This specific binding of xEFN-RBD3 and xEFN-RBD4 for EphA2, EphB2, and EphB3 indicating that these peptide may serve as competitive antagonists to endogenous or wild-type ephrin ligands produced by the subject.

### Example 15: Ephrin Ligand Mimetic Peptide Molecular Interactions with EphB2 and Competition with EphrinB1

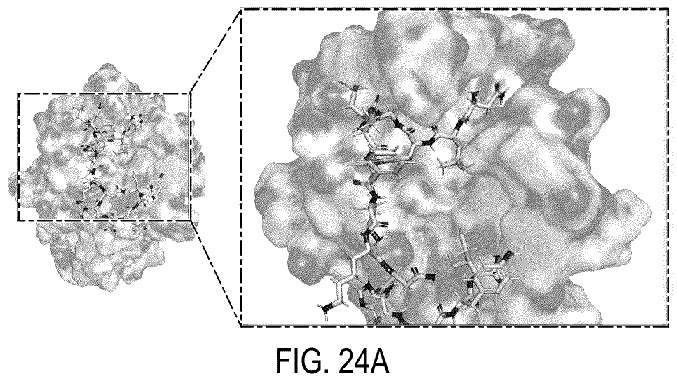

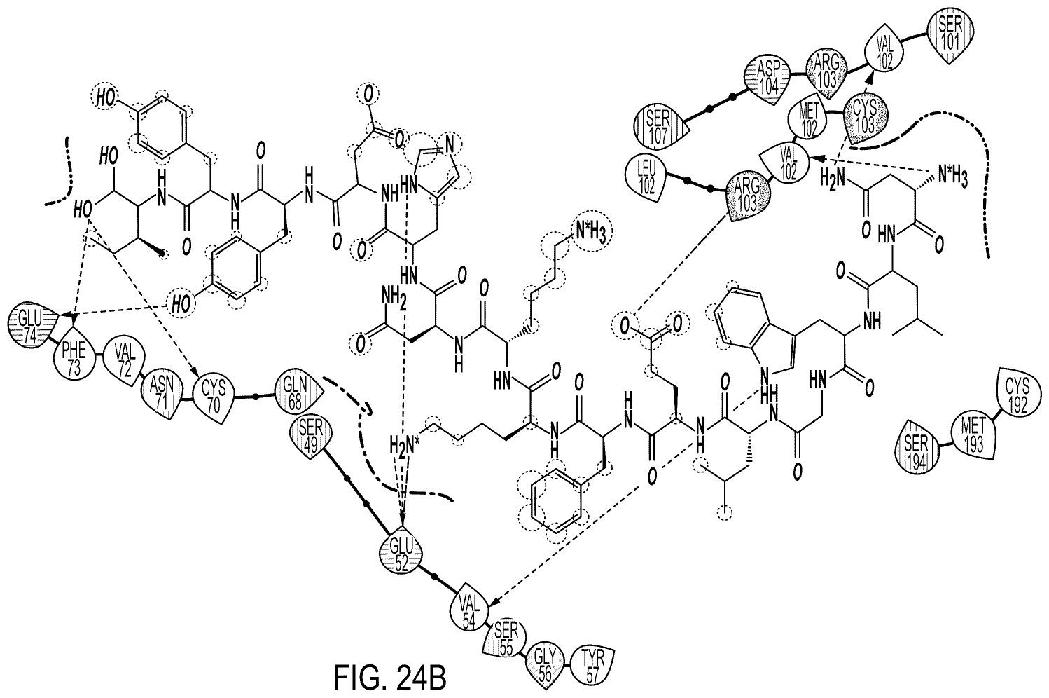

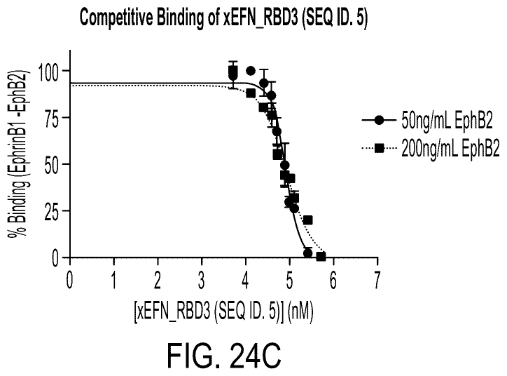

In this example xEFN_RBD3 was modeled for interactions with a resolved crystal structure of EphB2 {EphB2 (PDB: 2QBX)} solved by X-ray diffraction at 2.3 Å resolution (Chrencik J E et al. Three-dimensional structure of the EphB2 receptor in complex with an antagonistic peptide reveals a novel mode of inhibition. J Biol Chem. 2007-12-14: 282 (50): 36505-13). EphB2 (PDB: 2QBX) is a synthetic construct described in Chrencik J E et al. 2007. A docking model generated using the GLIDE docking software suite between xEFN_RBD3 and a resolved crystal structure for EphB2 is displayed in FIG. 24A. Key molecular interactions between xEFN_RBD3 and the EphB2 ligand binding pocket (LBP) are shown in the diagram of FIG. 24B and listed in Table 2 below. In Table 2, under interaction type, H-bond refers to a hydrogen bond and ionic refers to an ionic bond. Results were graphed of an in vitro binding assay displaying percent binding of EphB2 to ephrinB1 in the presence of an increasing concentration of xEFN_RBD3 as shown in FIG. 24C demonstrating a competitive antagonist action of xEFN_RBD3 in this molecular interaction Various concentrations of xEFN_RBD3 were shown to binding competitively to EphB2 in the presence of ephrinB1. Two different concentrations of EphB2 were tested (50 ng/ml and 200 ng/mL) to confirm this result at different receptor concentrations.

TABLE 2
Molecular interactions between xEFN_RBD3 (Seq ID. 3) and EphB2 (PDB: 2QBX)

| | | Receptor | Equivalent | | | |
|---|---|---|---|---|---|---|
| Peptide | | AA (PBD: | AA in | | Interaction | |
| AA | Peptide Atom | 2QBX) | NP_059145.2 | Receptor Atom | Type | Strength |
| ASN1 | NH side chain | VAL102 | VAL94 | C═O backbone | H-bond | 1.9 |
| ASN1 | NH backbone | VAL164 | VAL156 | C═O backbone | H-bond | 1.8 |
| TRP3 | NH indole | SER55 | SER47 | C═O backbone | H-bond | 2 |
| GLU6 | Oe2 side chain | ARG163 | ARG155 | NH2(+1) | Ionic | 1 |
| LYS8 | NH2+ | GLU52 | GLU44 | O(−) side chain | Ionic | 1 |
| LYS8 | NH2+ | GLU52 | GLU44 | O(−) side chain | H-bond | 1.8 |
| HIS11 | NE2-H indole | GLU52 | GLU44 | O(−) side chain | H-bond | 1.7 |
| TYR13 | OH-phenol | GLU74 | GLU66 | O(−) side chain | H-bond | 1.7 |
| ILE15 | C-term OH | CYS70 | CYS62 | C═O backbone | H-bond | 2.1 |
| ILE15 | C-term OH | PHE73 | PHE65 | NH backbone | H-bond | 3 |

In some embodiments, the amino acid sequence of the human ephrin type-B receptor 2 (EphB2) isoform is represented as EphB32 (pbd:2qbx). In some embodiments, the amino acid sequence of EphB2 (pbd:2qbx) is listed as part of SEQ ID NO: 100. In some embodiments, the amino acid sequence of the human ephrin type-B receptor 2 (EphB2) isoform is represented in NCBI Reference Sequence: NP_059145.2. The 986 amino acid sequence of NP_059145.2 is listed as SEQ ID NO: 101. In some embodiments, examples of EphB2 isoform sequences to which xEFN peptides may bind are included in Table 3. In some embodiments, the amino acid sequence of the human EphB2 isoform is represented in NCBI Reference Sequence: NP_004433.2. In some embodiments, the amino acid sequence of the human EphB2 isoform is represented in NCBI Reference Sequence: NP_001296121.1. In some embodiments, the amino acid sequence of the human EphB2 isoform is represented in NCBI Reference Sequence: NP_001296122.1. In some embodiments, the amino acid sequence of the human EphB2 isoform is represented in GenBank: AIC58934.1. In some embodiments, the amino acid sequence of the human EphB2 isoform is represented in GenBank: EAW95024.1. In some embodiments, the amino acid sequence of the human EphB2 isoform is represented in GenBank: EAW95023.1. In some embodiments, the amino acid sequence of the human EphB2 isoform is represented in NCBI Reference Sequence: XP_054191013.1. In some embodiments, the amino acid sequence of the human EphB2 isoform is represented in NCBI Reference Sequence: XP_054191014.1. In some embodiments, the amino acid sequence of the human EphB2 isoform is represented in NCBI Reference Sequence: XP_054191015.1. In some embodiments, the amino acid sequence of the human EphB2 isoform is represented in UniProt: P29323.

TABLE 3
Example EphB2 sequences with some ephrin ligand mimetic peptide molecular
interactions

| SEQ | |
|---|---|
| ID | |
| NO: | Amino Acid Sequence of EphB2 isoforms |
| 100 | MVSAIVLYVLLAAAAHSAFAAMVHHHHHHSAEETLMDSTTATAELGWMVHPPSGW |
| | EEVSGYDENMNTIRTYQVCNVFESSQNNWLRTKFIRRRGAHRIHVEMKFSVRDCSSI |
| | PSVPGSCKETFNLYYYEADFDSATKTFPNWMENPWVKVDTIAADESFSQVDLGGRV |
| | MKINTEVRSFGPVSRSGFYLAFQDYGGCMSLIAVRVFYRK |
| 101 | MALRRLGAALLLLPLLAAVEETLMDSTTATAELGWMVHPPSGWEEVSGYDENMNTI |
| | RTYQVCNVFESSQNNWLRTKFIRRRGAHRIHVEMKFSVRDCSSIPSVPGSCKETFNLY |
| | YYEADFDSATKTFPNWMENPWVKVDTIAADESFSQVDLGGRVMKINTEVRSFGPVS |
| | RSGFYLAFQDYGGCMSLIAVRVFYRKCPRIIQNGAIFQETLSGAESTSLVAARGSCIAN |
| | AEEVDVPIKLYCNGDGEWLVPIGRCMCKAGFEAVENGTVCRGCPSGTFKANQGDEA |
| | CTHCPINSRTTSEGATNCVCRNGYYRADLDPLDMPCTTIPSAPQAVISSVNETSLMLE |
| | WTPPRDSGGREDLVYNIICKSCGSGRGACTRCGDNVQYAPRQLGLTEPRIYISDLLAH |
| | TQYTFEIQAVNGVTDQSPFSPQFASVNITTNQAAPSAVSIMHQVSRTVDSITLSWSQPD |
| | QPNGVILDYELQYYEKELSEYNATAIKSPTNTVTVQGLKAGAIYVFQVRARTVAGYG |
| | RYSGKMYFQTMTEAEYQTSIQEKLPLIIGSSAAGLVFLIAVVVIAIVCNRRGFERADSE |
| | YTDKLQHYTSGHMTPGMKIYIDPFTYEDPNEAVREFAKEIDISCVKIEQVIGAGEFGE |
| | VCSGHLKLPGKREIFVAIKTLKSGYTEKQRRDFLSEASIMGQFDHPNVIHLEGVVTKS |
| | TPVMIITEFMENGSLDSFLRQNDGQFTVIQLVGMLRGIAAGMKYLADMNYVHRDLA |
| | ARNILVNSNLVCKVSDFGLSRFLEDDTSDPTYTSALGGKIPIRWTAPEAIQYRKFTSAS |
| | DVWSYGIVMWEVMSYGERPYWDMTNQDVINAIEQDYRLPPPMDCPSALHQLMLD |
| | CWQKDRNHRPKFGQIVNTLDKMIRNPNSLKAMAPLSSGINLPLLDRTIPDYTSFNTV |
| | DEWLEAIKMGQYKESFANAGFTSFDVVSQMMMEDILRVGVTLAGHQKKILNSIQVM |
| | RAQMNQIQSVEV |

### Aspects

Some embodiments relate to any of the following aspects:

Aspect 1: A composition comprising: an ephrin ligand mimetic peptide comprising an amino acid sequence at least 90% identical to a sequence selected from SEQ ID NOs: 1-6.

Aspect 2: The composition of aspect 1, wherein the ephrin ligand mimetic peptide comprises an amino acid sequence having one amino acid addition from a sequence selected from SEQ ID NOs: 1-6.

Aspect 3: The composition of aspect 1, wherein the ephrin ligand mimetic peptide comprises an amino acid sequence having one amino acid deletion from a sequence selected from SEQ ID NOs: 1-6.

Aspect 4: The composition of aspect 1, wherein the ephrin ligand mimetic peptide comprises an amino acid sequence having one or two amino acid substitutions from a sequence selected from SEQ ID NOs: 1-6.

Aspect 5: The composition of aspect 1, wherein the ephrin ligand mimetic peptide comprises an amino acid sequence selected from SEQ ID NOs: 1-6.

Aspect 6: The composition of aspect 1, wherein the ephrin ligand mimetic peptide comprises an amino acid sequence having one or two amino acid substitutions from a sequence selected from SEQ ID NOs: 1-2, or 4-6.

Aspect 7: The composition of aspect 1, wherein the ephrin ligand mimetic peptide comprises an amino acid sequence having one amino acid addition from a sequence selected from SEQ ID NOs: 1-2, or 4-6.

Aspect 8: The composition of aspect 1, wherein the ephrin ligand mimetic peptide comprises an amino acid sequence having one amino acid deletion from a sequence selected from SEQ ID NOs: 1-2, or 4-6.

Aspect 9: The composition of aspect 5, wherein the ephrin ligand mimetic peptide comprises the amino acid sequence selected from SEQ ID NOs: 1-2, or 4-6.

Aspect 10: The composition of aspect 1, wherein the ephrin ligand mimetic peptide comprises an amino acid sequence having one amino acid addition from a sequence selected from SEQ ID NOs: 4-6.

Aspect 11: The composition of aspect 1, wherein the ephrin ligand mimetic peptide comprises an amino acid sequence having one amino acid deletion from a sequence selected from SEQ ID NOs: 4-6.

Aspect 12: The composition of aspect 9, wherein the ephrin ligand mimetic peptide comprises the amino acid sequence selected from SEQ ID NOs: 4-6.

Aspect 13: The composition of aspect 9, wherein the ephrin ligand mimetic peptide comprises the amino acid sequence of SEQ ID NO: 1.

Aspect 14: The composition of aspect 9, wherein the ephrin ligand mimetic peptide comprises the amino acid sequence of SEQ ID NO: 2.

Aspect 15: The composition of aspect 5, wherein the ephrin ligand mimetic peptide comprises the amino acid sequence of SEQ ID NO: 3.

Aspect 16: The composition of aspect 12, wherein the ephrin ligand mimetic peptide comprises the amino acid sequence of SEQ ID NO: 4.

Aspect 17: The composition of aspect 12, wherein the ephrin ligand mimetic peptide comprises the amino acid sequence of SEQ ID NO: 5.

Aspect 18: The composition of aspect 12, wherein the ephrin ligand mimetic peptide comprises the amino acid sequence of SEQ ID NO: 6.

Aspect 19: The composition of aspect 13, wherein the ephrin ligand mimetic peptide consists essentially of the amino acid sequence of SEQ ID NO: 1.

Aspect 20: The composition of aspect 14, wherein the ephrin ligand mimetic peptide consists essentially of the amino acid sequence of SEQ ID NO: 2.

Aspect 21: The composition of aspect 15, wherein the ephrin ligand mimetic peptide consists essentially of the amino acid sequence of SEQ ID NO: 3.

Aspect 22: The composition of aspect 16, wherein the ephrin ligand mimetic peptide consists essentially of the amino acid sequence of SEQ ID NO: 4.

Aspect 23: The composition of aspect 17, wherein the ephrin ligand mimetic peptide consists essentially of the amino acid sequence of SEQ ID NO: 5.

Aspect 24: The composition of aspect 18, wherein the ephrin ligand mimetic peptide consists essentially of the amino acid sequence of SEQ ID NO: 6.

Aspect 25: The composition of any one of aspect 7-24, wherein the ephrin ligand mimetic peptide targets a ligand binding site on one or more Eph receptors.

Aspect 26: The composition of any one of aspect 7-24, wherein the ephrin ligand mimetic peptide targets a plurality of ligand binding sites on one or more Eph receptors.

Aspect 27: The composition of aspect 25, wherein the ephrin ligand mimetic peptide competitively targets one or more ligand binding sites on one or more Eph receptors.

Aspect 28: The composition of aspect 27, wherein competitive targeting comprises ephrin ligand mimetic peptide binding to one or more Eph receptors with a higher binding affinity than a natural ephrin ligand.

Aspect 29: The composition of aspect 27 or 28, wherein the ephrin ligand mimetic peptide binds to EphB2 at any of GLU44, SER47, CYS62, PHE65, GLU66, VAL94, ARG155, or VAL156 in relation to SEQ ID NO: 101.

Aspect 30: The composition of aspect 17, wherein the ephrin ligand mimetic peptide binds to EphB2 at any of GLU44, SER47, CYS62, PHE65, GLU66, VAL94, ARG155, or VAL156 in relation to SEQ ID NO: 101.

Aspect 31: The composition of aspect 23, wherein the ephrin ligand mimetic peptide binds to EphB2 at any of GLU44, SER47, CYS62, PHE65, GLU66, VAL94, ARG155, or VAL156 in relation to SEQ ID NO: 101.

Aspect 32: The composition of aspect 31, wherein the natural ephrin ligand is an ephrin-A ligand and wherein the one or more Eph receptors is selected from the group consisting of EphA1, EphA2, EphA3, EphA4, EphA5, EphA6, EphA7, EphA8, and EphA10.

Aspect 33: The composition of aspect 31, wherein the natural ephrin ligand is an ephrin-B ligand and wherein the one or more Eph receptors is selected from the group consisting of EphB1, EphB2, EphB3, EphB4, and EphB6.

Aspect 34: The composition of aspect 32, wherein the ephrin-A ligand is ephrin-A1, ephrin-A2, ephrin-A3, ephrin-A4, or ephrin-A5.

Aspect 35: The composition of aspect 32, wherein the ephrin-A ligand is human ephrin-A1, human ephrin-A2, human ephrin-A3, human ephrin-A4, or human ephrin-A5.

Aspect 36: The composition of aspect 33, wherein the ephrin-B ligand is ephrin-B1, ephrin-B2, or ephrin-B3.

Aspect 37: The composition of aspect 33, wherein the ephrin-B ligand is human ephrin-B1, human ephrin-B2, or human ephrin-B3.

Aspect 38: The composition of any one of aspect 10-24, wherein the ephrin ligand mimetic peptide mimics one or more receptor binding domains (RBD) in one or more ephrin ligands to compete with the binding of natural ephrin ligands.

Aspect 39: The composition of any one of aspect 10-24, wherein the ephrin ligand mimetic peptide mimics one or more receptor binding domains (RBD) found in hEfnA1, hEfnA2, hEfnA3, hEfnA4, hEfnA5, hEfnB1, hEfnB2, or hEfnB3.

Aspect 40: The composition of any one of aspect 10-24, wherein the ephrin ligand mimetic peptide mimics two or more receptor binding domains (RBD) found in the group consisting of hEfnA1, hEfnA2, hEfnA3, hEfnA4, hEfnA5, hEfnB1, hEfnB2, and hEfnB3.

Aspect 41: The composition of any one of aspect 10-24, wherein the ephrin ligand mimetic peptide mimics three or more receptor binding domains (RBD) found in the group consisting of hEfnA1, hEfnA2, hEfnA3, hEfnA4, hEfnA5, hEfnB1, hEfnB2, and hEfnB3.

Aspect 42: The composition of any one of aspect 10-24, wherein the ephrin ligand mimetic peptide mimics four or more receptor binding domains (RBD) found in the group consisting of hEfnA1, hEfnA2, hEfnA3, hEfnA4, hEfnA5, hEfnB1, hEfnB2, and hEfnB3.

Aspect 43: The composition of any one of aspect 10-24, wherein the ephrin ligand mimetic peptide mimics receptor binding domains (RBD) found in hEfnA1, hEfnA2, hEfnA3, hEfnA4, hEfnA5, hEfnB1, hEfnB2, and hEfnB3.

Aspect 44: The composition of any one of aspect 10-24, wherein the ephrin ligand mimetic peptide functions as an antagonist to one or more Eph receptors wherein the one or more Eph receptors are selected from EphA1, EphA2, EphA3, EphA4, EphA5, EphA6, EphA7, EphA8, EphA10, EphB1, EphB2, EphB3, EphB4, and EphB6.

Aspect 45: The composition of any one of aspect 10-24, wherein the ephrin ligand mimetic peptide functions as an antagonist to two or more Eph receptors wherein the two or more Eph receptors are selected from EphA1, EphA2, EphA3, EphA4, EphA5, EphA6, EphA7, EphA8, EphA10, EphB1, EphB2, EphB3, EphB4, and EphB6.

Aspect 46: The composition of any one of aspect 10-24, wherein the ephrin ligand mimetic peptide functions as an antagonist to EphA2 and EphB2.

Aspect 47: The composition of any one of aspect 10-24, wherein the ephrin ligand mimetic peptide functions as an antagonist to three or more Eph receptors wherein the three or more Eph receptors are selected from EphA1, EphA2, EphA3, EphA4, EphA5, EphA6, EphA7, EphA8, EphA10, EphB1, EphB2, EphB3, EphB4, and EphB6.

Aspect 48: The composition of any one of aspect 10-24, wherein the ephrin ligand mimetic peptide functions as an antagonist to four or more Eph receptors wherein the four or more Eph receptors are selected from EphA1, EphA2, EphA3, EphA4, EphA5, EphA6, EphA7, EphA8, EphA10, EphB1, EphB2, EphB3, EphB4, and EphB6.

Aspect 49: The composition of aspect 44, wherein the ephrin ligand mimetic peptide reduces an extent of phosphorylation of one or more Eph receptors following administering of the composition to a subject.

Aspect 50: The composition of aspect 49, wherein the phosphorylation of one or more Eph receptors is reduced by at least 10%, 20%, 30%, 40%, 50%, 60%, 70%, 80%, or 90%.

Aspect 51: The composition of aspect 44, wherein the ephrin ligand mimetic peptide reduces expression of one or more Eph receptors following administering of the composition to a subject.

Aspect 52: The composition of aspect 51, wherein the expression of EphA2 and EphB2 are reduced following administering of the composition to the subject.

Aspect 53: The composition of aspect 44, wherein the ephrin ligand mimetic peptide increases regeneration of axonal projections by >500 μm distal to a crush site on a nerve.

Aspect 54: The composition of aspect 44, wherein the ephrin ligand mimetic peptide induces neurite sprouting following administering of the composition to the subject.

Aspect 55: The composition of aspect 54, wherein the ephrin ligand mimetic peptide induces local neurite sprouting following administering of the composition locally to the subject.

Aspect 56: The composition of aspect 44, wherein the ephrin ligand mimetic peptide induces neurite sprouting in retinal ganglion cells (RGCs) following administering of the composition intravitreally to the subject.

Aspect 57: The composition of aspect 44, wherein the ephrin ligand mimetic peptide protects a plurality of neurons from neurodegeneration following administering of the composition to a subject.

Aspect 58: A method of treating a neurodegenerative disorder in a subject in need thereof, the method comprising administering to the subject an effective amount of a composition of any one of aspect 7-24.

Aspect 59: The method of aspect 58, wherein the neurodegenerative disorder comprises Alzheimer's disease (AD), traumatic brain injury (TBI), spinal cord injury (SCI), neuropathy, retinopathy, optic neuropathy, glaucoma, glaucomatous degeneration of the optic nerve and retina, age-related macular degeneration (AMD), or stroke.

Aspect 60: The method of aspect 58, wherein the neurodegenerative disorder comprises Alzheimer's disease, Pick's disease, Niemann-Pick disease type C, Frontal temporal dementia (FTD), frontotemporal lobar degeneration, chronic traumatic encephalopathy (CTE), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, or Hallevorden-Spatz disease.

Aspect 61: The method of aspect 58, wherein the neurodegenerative disorder comprises Alzheimer's disease.

Aspect 62: The method of aspect 58, wherein the neurodegenerative disorder comprises glaucoma Aspect 63: The method of any one of aspect 59-62, wherein the composition is administered systemically or locally.

Aspect 64: The method of aspect 63, wherein the composition is administered is administered intravenously, intramuscularly, intrathecally, intracerebrally, subcutaneously, orally, nasally, topically, buccally, or sublingually.

Aspect 65: The method of aspect 63, wherein the composition is administered directly to the CNS of the subject via intravenous delivery, intravascular delivery, intrathecal delivery, intracisternal delivery, intraspinal delivery, subpial delivery, or intracerebroventricular delivery.

Aspect 66: The method of aspect 63, wherein the composition is administered via stereotaxic injection into the brain parenchyma or the spinal cord parenchyma.

Aspect 67: The method of aspect 63, wherein the composition is administered via stereotaxic injection into one or a plurality of regions of cerebral cortex, entorhinal cortex, hippocampus, thalamus, mammillary body, amygdala, or basal ganglia.

Aspect 68: The method of aspect 63, wherein the composition is administered to an eye of the subject via intravitreal injection.

Aspect 69: The method of any one of aspect 59-62 or 64-68, wherein the subject is at a prodromal stage of the neurodegenerative disorder.

Aspect 70: The method of any one of aspect 59-62 or 64-68, wherein an extent of phosphorylation of one or more Eph receptors is reduced following the administering.

Aspect 71: The method of aspect 70, wherein the extent of phosphorylation of one or more Eph receptors is reduced by at least 10%, 20%, 30%, 40%, 50%, 60%, 70%, 80%, or 90%.

Aspect 72: The method of aspect 70, wherein the extent of phosphorylation of EphA2 and EphB2 is reduced by at least 10%, 20%, 30%, 40%, 50%, 60%, 70%, 80%, or 90%.

Aspect 73: The method of any one of aspect 59-62 or 64-68, or 71-72, wherein the administering prevents a progression of a neurodegenerative disorder in the subject.

Aspect 74: The method of any one of aspect 59-62 or 64-68, or 71-72, wherein the administering provides neuroprotection from a further extent of pathological neuronal cell loss in the subject.

Aspect 75: The method of any one of aspect 59-62 or 64-68, or 71-72, wherein the administering stabilizes a progression of a neurodegenerative disorder in the subject.

Aspect 76: The method of any one of aspect 59-62 or 64-68, or 71-72, wherein the administering improves one or more symptoms of a neurodegenerative disorder in the subject.

Aspect 77: The method of any one of aspect 59-62 or 64-68, or 71-72, wherein the administering increases regeneration of axonal projections by >500 μm distal to a crush site on a nerve.

Aspect 78: An engineered protein, comprising: an amino acid sequence at least 85% identical to any one of SEQ ID NOs: 1-6.

Aspect 79: The engineered protein of aspect 78, wherein the amino acid sequence is at least 85% identical to SEQ ID NO: 4, 5, or 6.

Aspect 80: The engineered protein of aspect 78, wherein the amino acid sequence is 100% identical to SEQ ID NO: 4, 5, or 6.

Aspect 81: An engineered protein, comprising: the amino acid sequence of any one of SEQ ID NOs: 1-6, or a sequence thereof having 1, 2, 3, or 4 amino acid substitutions, additions, or deletions.

Aspect 82: The engineered protein of aspect 81, wherein the amino acid sequence comprises the amino acid sequence of SEQ ID NO: 4, 5, or 6, or a sequence thereof having 1, 2, 3, or 4 amino acid substitutions, additions, or deletions.

Aspect 83: The engineered protein of aspect 81, wherein the amino acid sequence comprises the amino acid sequence of SEQ ID NO: 4, 5, or 6, or a sequence thereof having 1 or 2 amino acid substitutions, additions, or deletions.

Aspect 84: The engineered protein of aspect 80, wherein the amino acid sequence comprises the amino acid sequence of SEQ ID NO: 4.

Aspect 85: The engineered protein of aspect 80, wherein the amino acid sequence comprises the amino acid sequence of SEQ ID NO: 5.

Aspect 86: The engineered protein of aspect 80, wherein the amino acid sequence comprises the amino acid sequence of SEQ ID NO: 6.

Aspect 87: The engineered protein of aspect 82 or 85, wherein an amino acid ASN at an aligned position 1 of SEQ ID NO: 5 interacts with a VAL residue in an EphB2 receptor at an aligned position of VAL94 from SEQ ID NO: 101.

Aspect 88: The engineered protein of aspect 82 or 85, wherein an amino acid ASN at an aligned position 1 of SEQ ID NO: 5 interacts with a VAL residue in an EphB2 receptor at an aligned position of VAL156 from SEQ ID NO: 101.

Aspect 89: The engineered protein of aspect 82 or 85, wherein an amino acid TRP at an aligned position 3 of SEQ ID NO: 5 interacts with a SER residue in an EphB2 receptor at an aligned position of SER47 from SEQ ID NO: 101.

Aspect 90: The engineered protein of aspect 82 or 85, wherein an amino acid GLU at an aligned position 6 of SEQ ID NO: 5 interacts with a ARG residue in an EphB2 receptor at an aligned position of ARG155 from SEQ ID NO: 101.

Aspect 91: The engineered protein of aspect 82 or 85, wherein an amino acid LYS at an aligned position 8 of SEQ ID NO: 5 interacts with a GLU residue in an EphB2 receptor at an aligned position of GLU44 from SEQ ID NO: 101.

Aspect 92: The engineered protein of aspect 82 or 85, wherein an amino acid HIS at an aligned position 11 of SEQ ID NO: 5 interacts with a GLU residue in an EphB2 receptor at an aligned position of GLU44 from SEQ ID NO: 101.

Aspect 93: The engineered protein of aspect 82 or 85, wherein an amino acid TYR at an aligned position 13 of SEQ ID NO: 5 interacts with a GLU residue in an EphB2 receptor at an aligned position of GLU66 from SEQ ID NO: 101.

Aspect 94: The engineered protein of aspect 82 or 85, wherein an amino acid ILE at an aligned position 15 of SEQ ID NO: 5 interacts with a CYS residue in an EphB2 receptor at an aligned position of CYS62 from SEQ ID NO: 101.

Aspect 95: The engineered protein of aspect 82 or 85, wherein an amino acid ILE at an aligned position 15 of SEQ ID NO: 5 interacts with a PHE residue in an EphB2 receptor at an aligned position of PHE65 from SEQ ID NO: 101.

Aspect 96: The engineered protein of aspect 78, wherein the amino acid sequence comprises the amino acid sequence of SEQ ID NO: 4, 5, or 6.

Aspect 97: The engineered protein of any one of aspect 78-86 wherein the engineered protein binds an ephrin receptor.

Aspect 98: A pharmaceutical composition, comprising the engineered protein of any one of aspect 78-97, and a pharmaceutically acceptable carrier.

Aspect 99: A method of treatment, comprising administering an effective amount of the pharmaceutical composition of aspect 98 to a subject in need thereof.

Aspect 100: A method of binding an ephrin receptor, comprising contacting the ephrin receptor with the engineered protein of any one of aspect 78-97, thereby binding the ephrin receptor.

Aspect 101: The method of aspect 100, wherein the ephrin receptor is in or on a cell.

Aspect 102: A method of inhibiting activity of an ephrin receptor, comprising contacting the ephrin receptor with the engineered protein of any one of aspect 78-97, thereby inhibiting the activity of the ephrin receptor.

Aspect 103: The method of aspect 102, wherein the ephrin receptor is in or on a cell.

Aspect 104: A method of reducing expression of an ephrin receptor, comprising contacting a cell with the ephrin receptor with the engineered protein of any one of aspect 78-97.

Aspect 105: An in vivo modified protein, comprising: an ephrin receptor bound to a ligand comprising an ephrin ligand mimetic peptide.

Aspect 106: The in vivo modified protein of aspect 105, wherein the ephrin ligand mimetic peptide comprises the engineered protein of any one of aspect 78-97.

Aspect 107: A method of treating or preventing a neurodegenerative disease in a subject comprising administering a therapeutically effective amount of one or more pan-Eph receptor Ephrin ligand mimetic peptides to treat or prevent neurodegenerative disease in the subject.

Aspect 108: The method of aspect 107, wherein the neurodegenerative disease is Alzheimer's disease (AD), traumatic brain injury (TBI), spinal cord injury (SCI), neuropathy, retinopathy, optic neuropathy, glaucoma/glaucomatous degeneration of the optic nerve and retina, stroke or other Central Nervous System (CNS) neurodegenerative diseases.

Aspect 109: The method of aspect 107 or 108, wherein the Ephrin ligand mimetic peptide competitively targets ligand binding sites on the Eph receptor.

Aspect 110: The method of aspect 109, wherein the Ephrin ligand mimetic peptide mimics receptor binding domains (RBD) in Ephrin (efn) ligands to compete with the binding of Efn ligands.

Aspect 111: The method of aspect 109 wherein the Ephrin ligand mimetic peptide antagonizes the activation of both EphA and EphB class receptors.

Aspect 112: The method of aspect 110, wherein the Ephrin ligand mimetic peptide increases regeneration of axonal projections by >500 μm distal to a crush site on a nerve.

Aspect 113: The method of aspect 110, wherein the Ephrin ligand mimetic peptide reduces expression of one or more Eph receptors.

Aspect 114: A composition comprising a pan-Eph receptor Ephrin ligand mimetic peptide.

Aspect 115: The composition of aspect 114, wherein the Ephrin ligand mimetic peptide competitively targets ligand binding sites on the Eph receptor.

Aspect 116: The composition of aspect 114 or 115, wherein the Ephrin ligand mimetic peptide antagonizes both EphA and EphB receptor activation.

Aspect 117: The composition of aspect 114 or 115, wherein the Ephrin ligand mimetic peptide reduces expression of one or more Eph receptors.

Aspect 118: The composition of aspect 116, wherein the Ephrin ligand mimetic peptide is selected from the group consisting of SEQ ID NO: 1, SEQ ID NO: 2, SEQ ID NO: 3, SEQ ID NO: 4, SEQ ID NO: 5, or SEQ ID NO: 6.

Aspect 119: A composition comprising: an ephrin ligand mimetic peptide comprising an amino acid sequence of ASN-Xaa1-TRP-GLY-Xaa2-GLU-PHE-LYS-Xaa3-Xaa4-HIS-Xaa5-TYR-Xaa6-ILE;
wherein Xaa1 is THR, SER, TYR, or LEU;
wherein Xaa2 is LYS, PHE, TYR, LEU, or HIS;
wherein Xaa3 is GLU, PRO, ALA, LYS, or SER;
wherein Xaa4 is GLY, HIS, or ASN;
wherein Xaa5 is SER, GLU, THR, or ASP; and
wherein Xaa6 is TYR or PHE.

Aspect 120: The composition of aspect 119, wherein an amino acid ASN at an aligned position 1 of SEQ ID NO: 5 interacts with a VAL residue in an EphB2 receptor at an aligned position of VAL94 from SEQ ID NO: 101.

Aspect 121: The composition of aspect 119, wherein an amino acid ASN at an aligned position 1 of SEQ ID NO: 5 interacts with a VAL residue in an EphB2 receptor at an aligned position of VAL156 from SEQ ID NO: 101.

Aspect 122: The composition of aspect 119, wherein an amino acid TRP at an aligned position 3 of SEQ ID NO: 5 interacts with a SER residue in an EphB2 receptor at an aligned position of SER47 from SEQ ID NO: 101.

Aspect 123: The composition of aspect 119, wherein an amino acid GLU at an aligned position 6 of SEQ ID NO: 5 interacts with a ARG residue in an EphB2 receptor at an aligned position of ARG155 from SEQ ID NO: 101.

Aspect 124: The composition of aspect 119, wherein an amino acid LYS at an aligned position 8 of SEQ ID NO: 5 interacts with a GLU residue in an EphB2 receptor at an aligned position of GLU44 from SEQ ID NO: 101.

Aspect 125: The composition of aspect 119, wherein an amino acid HIS at an aligned position 11 of SEQ ID NO: 5 interacts with a GLU residue in an EphB2 receptor at an aligned position of GLU44 from SEQ ID NO: 101.

Aspect 126: The composition of aspect 119, wherein an amino acid TYR at an aligned position 13 of SEQ ID NO: 5 interacts with a GLU residue in an EphB2 receptor at an aligned position of GLU66 from SEQ ID NO: 101.

Aspect 127: The composition of aspect 119, wherein an amino acid ILE at an aligned position 15 of SEQ ID NO: 5 interacts with a CYS residue in an EphB2 receptor at an aligned position of CYS62 from SEQ ID NO: 101.

Aspect 128: The composition of aspect 119, wherein an amino acid ILE at an aligned position 15 of SEQ ID NO: 5 interacts with a PHE residue in an EphB2 receptor at an aligned position of PHE65 from SEQ ID NO: 101.

Aspect 129: The composition any one of aspect 119-128, wherein the ephrin ligand mimetic peptide forms an in vivo bound protein complex by binding to one or more ephrin receptors.

Aspect 130: The composition any one of aspect 119-128, wherein the ephrin ligand mimetic peptide antagonizes both EphA and EphB receptor activation.

Aspect 131: The composition any one of aspect 119-128, wherein the ephrin ligand mimetic peptide mimics one or more receptor binding domains (RBD) in one or more ephrin ligands to compete with the binding of natural ephrin ligands.

Aspect 132: The composition any one of aspect 119-128, wherein the ephrin ligand mimetic peptide functions as an antagonist to EphA2, EphB2 and EphB3.

Aspect 133: The composition any one of aspect 119-128, wherein the ephrin ligand mimetic peptide functions as an antagonist to EphA2 and EphB2.

Aspect 134: The composition any one of aspect 119-128, wherein the ephrin ligand mimetic peptide reduces an extent of phosphorylation of one or more Eph receptors following administering of the composition to a subject.

Aspect 135: The composition any one of aspect 119-128, wherein the ephrin ligand mimetic peptide reduces expression of one or more Eph receptors following administering of the composition to a subject.

Aspect 136: The composition any aspect 135, wherein the expression of EphA2 and EphB2 are reduced following administering of the composition to the subject.

Aspect 137: The composition any one of aspect 119-128, wherein the ephrin ligand mimetic peptide increases regeneration of axonal projections by >500 μm distal to a crush site on a nerve.

Aspect 138: The composition any one of aspect 119-128, wherein the ephrin ligand mimetic peptide induces neurite sprouting following administering of the composition to the subject.

Aspect 139: The composition any one of aspect 119-128, wherein the ephrin ligand mimetic peptide protects a plurality of neurons from neurodegeneration following administering of the composition to a subject.

Aspect 140: A method of treating or preventing cancer in a subject comprising administering a therapeutically effective amount of one or more pan-Eph receptor Ephrin ligand mimetic peptides to treat or prevent cancer in the subject.

What is claimed is:

1. An engineered protein, comprising the amino acid sequence: ASN-Xaa1-TRP-GLY-Xaa2-GLU-PHE-LYS-Xaa3-Xaa4-HIS-Xaa5-TYR-Xaa6-ILE, wherein
Xaa1 is THR, SER TYR, or LEU;
Xaa2 is LYS, PHE, TYR, LEU, or HIS;
Xaa3 is GLU, PRO, ALA, LYS, or SER;
Xaa4 is GLY, HIS, or ASN;
Xaa5 is SER, GLU, THR, or ASP; and
Xaa6 is TYR or PHE.

2. The engineered protein of claim 1, wherein the amino acid sequence is at least 80% identical, or at least 90% identical to SEQ ID NO: 5.

3. The engineered protein of claim 1, wherein the amino acid sequence is identical to SEQ ID NO: 5.

4. The engineered protein of claim 1, wherein the engineered protein is 10-50 amino acids long.

5. The engineered protein of claim 1, wherein the engineered protein is 12-20 amino acids long.

6. The engineered protein of claim 1, wherein the engineered protein is about 15 amino acids long.

7. The engineered protein of claim 1, wherein the engineered protein is 15 amino acids long.

8. The engineered protein of claim 1, wherein the engineered protein binds to an ephrin receptor.

9. An engineered protein, comprising: an amino acid sequence at least 80% identical, or at least 90% identical to any one of SEQ ID NOs: 1-6.

10. The engineered protein of claim 9, wherein the amino acid sequence is identical to any one of SEQ ID NOs: 1-6.

11. The engineered protein of claim 9, wherein the engineered protein is 10-50 amino acids long.

12. The engineered protein of claim 9, wherein the engineered protein is 12-20 amino acids long.

13. The engineered protein of claim 9, wherein the engineered protein is about 15 amino acids long.

14. The engineered protein of claim 9, wherein the engineered protein is 15 amino acids long.

15. The engineered protein of claim 9, wherein the engineered protein binds to an ephrin receptor.

16. A pharmaceutical composition, comprising the engineered protein of any one of claims 1-15, and a pharmaceutically acceptable carrier.

17. A method of treatment, comprising administering the pharmaceutical composition of claim 16 to a subject.

18. The method of claim 17, wherein the pharmaceutical composition, when administered in an effective amount, treats a neurodegenerative disorder in the subject.

19. The method of claim 17, wherein the pharmaceutical composition, when administered in an effective amount, treats an eye disorder in the subject.

20. The method of claim 19, wherein the eye disorder comprises glaucoma.

21. The method of claim 17, wherein the pharmaceutical composition, when administered in an effective amount, reduces intraocular pressure in an eye of the subject.

22. The method of claim 21, wherein the intraocular pressure is reduced by at least 10%, relative to a baseline intraocular pressure measurement.
